SEQUENCE LISTING (4 sequences)

SEQ ID NO: 1 - DNA, 2359 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 2 - protein, 294 aa, Artificial Sequence
  source: /note=“Description of Artificial Sequence Synthetic polypeptide”
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 3 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - DNA, 1621 bp, Homo sapiens
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactcttttgaaaaaccatctgtaaaagactggggtgggggtgggaggccagcacggtggtgaggcagttgagaaaatttgaatgtggattagattttgaatgatattggataattattggtaattttatggcctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaattttttttta

(19) United States
(12) Patent Application Publication
(10) Pub. No.: US 2022/0001028 A1
(43) Pub. Date: Jan. 6, 2022

(54) AAV VIRAL VECTORS AND USES THEREOF

(71) Applicant: NOVARTIS AG, Basel (CH)

(72) Inventors: James Michael HATFIELD, Bannockburn, IL (US); Robert Emil HODGE, Bannockburn, IL (US); Douglas FELTNER, Bannockburn, IL (US); Joseph BALLEYDIER, Bannockburn, IL (US); Matthew MERIGGIOLI, Bannockburn, IL (US); Brian K. KASPAR, New Albany, OH (US); Allan Arman KASPAR, Carlsbad, CA (US)

(21) Appl. No.: 17/309,403

(22) PCT Filed: Nov. 27, 2019
(86) PCT No.: PCT/US2019/063649

§ 371 Date: May 25, 2021

Related U.S. Application Data
(60) Provisional application No. 62/773,894, filed on Nov. 30, 2018.
(60) Provisional application No. 62/835,242, filed on Apr. 17, 2019.

(51) Int. Cl. A61K 48/00; C12N 15/86

(52) U.S. Cl. CPC A61K 48/005; C12N 2750/14143; C12N 15/86

(57) ABSTRACT

Disclosed herein are compositions comprising AAV9 viral vectors and methods of using them to treat SMA patients, e.g., Type II and Type III Spinal Muscular Atrophy (SMA) patients.

### RELATED APPLICATIONS

This application claims priority to U.S. Provisional Patent Application No. 62/773,894, filed Nov. 30, 2018, and U.S. Provisional Patent Application No. 62/835,242, filed Apr. 17, 2019. The contents of these applications are herein incorporated by reference in their entirety.

### SEQUENCE LISTING

The instant application contains a Sequence Listing which has been submitted electronically in ASCII format and is hereby incorporated by reference in its entirety. Said ASCII copy, created on Nov. 12, 2019, is named 14452_0025-00304_SL.txt and is 14,833 bytes in size.

### FIELD OF THE DISCLOSURE

This disclosure relates to compositions and uses of viral particles.

### BACKGROUND

Adeno-associated virus (AAV) is a member of the parvoviridae family. The AAV genome comprises a linear single-stranded DNA molecule approximately 4.7 kilobases (kb) in length having two major open reading frames encoding the non-structural Rep (replication) and structural Cap (capsid) proteins. Flanking the AAV coding regions are two cis-acting inverted terminal repeat (ITR) sequences, approximately 145 nucleotides in length, with interrupted palindromic sequences that can fold into hairpin structures that function as primers during initiation of DNA replication. In addition to their role in DNA replication, the ITR sequences have been shown to play a role in viral integration, rescue from the host genome, and encapsidation of viral nucleic acid into mature virions (Muzyczka, (1992) Curr. Top. Micro. Immunol. 158:97-129).

Multiple serotypes of AAV exist and offer varied tissue tropism. Known serotypes include, for example, AAV1, AAV2, AAV3, AAV4, AAV5, AAV6, AAV7, AAV8, AAV9, AAV10 and AAV11. AAV9 is described in U.S. Pat. No. 7,198,951 and in Gao et al., J. Virol., 78: 6381-6388 (2004), which are hereby incorporated by reference in their entirety. Advances in the delivery of AAV6 and AAV8 have made possible the transduction by these serotypes of skeletal and cardiac muscle following simple systemic intravenous or intraperitoneal injections. See Pacak et al., Circ. Res., 99(4): 3-9 (2006) and Wang et al., Nature Biotech. 23(3): 321-8 (2005). The use of AAV to target cell types within the central nervous system, though, has required surgical intraparenchymal injection. See Kaplitt et al., “Safety and tolerability of gene therapy with an adeno-associated virus (AAV) borne GAD gene for Parkinson's disease: an open label, phase I trial.” Lancet, 369:2097-2105; Marks et al., “Gene delivery of AAV2-neurturin for Parkinson's disease: a double-blind, randomized, controlled trial.” Lancet Neurol 9:1164-1172; and Worgall et al., “Treatment of late infantile neuronal ceroid lipofuscinosis by CNS administration of a serotype 2 adeno-associated virus expressing CLN2 cDNA.” Hum Gene Ther, 19(5):463-74.

The nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, e.g., interferon-mediated responses; (iii) wild-type viruses have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected).

Self-complementary adeno-associated vectors (scAAV) are viral vectors engineered from the naturally occurring adeno-associated virus (AAV) for use in gene therapy. ScAAV is termed “self-complementary” because the coding region has been designed to form an intramolecular double-stranded DNA template. A rate-limiting step for the standard AAV genome life cycle involves the second-strand synthesis since the typical AAV genome is a single-stranded DNA template. However, this is not the case for scAAV genomes. Upon infection, rather than waiting for cell mediated synthesis of the second strand, the two complementary halves of scAAV will associate to form one double stranded DNA (dsDNA) unit that is ready for immediate replication and transcription.

Spinal muscular atrophy (SMA) is a neurogenetic disorder caused by a loss or mutation in the survival motor neuron 1 gene (SMN1) on chromosome 5q13, which leads to reduced SMN protein levels and a selective dysfunction of motor neurons. SMA is an autosomal recessive, early childhood disease with an incidence of 1:10,000 live births. Sugarman et al., “Pan-ethnic carrier screening and prenatal diagnosis for spinal muscular atrophy: clinical laboratory analysis of >72,400 specimens.” European journal of human genetics, 20(1): 27-32. All forms of SMA are autosomal recessive in inheritance and are caused by deletions or mutations of the survival motor neuron 1 (SMN1) gene. Humans also carry a second nearly identical copy of the SMN1 gene called SMN2. Both the SMN1 and SMN2 genes express SMN protein, however, the amount of functional full-length protein produced by SMN2 is much less (by 10-15%) than that produced by SMN1. Although SMN2 cannot completely compensate for the loss of the SMN1 gene, patients with milder forms of SMA generally have higher SMN2 copy numbers. In a large early study by Feldkotter et al., 2 copies of SMN2 was 97% predictive for developing SMA Type I, 3 copies of SMN2 was 83% predictive for developing SMA Type II, and 4 copies of SMN2 was 84% predictive of SMA Type III. Feldkotter et al., “Quantitative analyses of SMN1 and SMN2 based on real-time lightCycler PCR: fast and highly reliable carrier testing and prediction of severity of spinal muscular atrophy.” American Journal of Human Genetics, 70(2): 358-368. As these percentages do not reflect the possible impact of modifier mutations, they may understate the relationship between copy number (in the absence of a genetic modifier) and clinical phenotype. Among 113 patients with Type I SMA, 9 with one SMN2 copy lived <11 months, 88/94 with two SMN2 copies lived <21 months, and 8/10 with three SMN2 copies lived 33-66 months

Type I SMA is the leading cause of infant mortality due to genetic diseases. Disease severity and clinical prognosis depends on the number of copies of SMN2. In its most common and severe form (Type I), hypotonia and progressive weakness are recognized in the first few months of life, leading to diagnosis by 6 months of age and then death due to respiratory failure by age two. SMA Type I is the leading genetic cause of infant death. Motor neuron loss in SMA Type I is profound in the early postnatal period (or may even start in the pre-natal period), and patients never attain independent sitting. Type I SMA patients typically have 1 or 2 copies of the SMN2 gene. In contrast, Type II SMA manifests within the first 18 months, and children afflicted with this condition are able to maintain sitting unassisted but never walk independently. Type II SMA patients typically have 3 copies of the SMN2 gene. SMA Type III patients attain the ability to walk unaided. Under the Type III rubric, Type IIIa patients usually show onset of disease at <3 years of age while Type IIIb patients have onset after 3 years of age. Motor neurons in Type II and III SMA patients appear to adapt and compensate during development and persist into adult life. Type III SMA patients typically have 3 or 4 copies of the SMN2 gene. The findings from various neurophysiological and animal studies have shown an early loss of motor neurons in the embryonic and early postnatal periods. Swoboda et al., “Natural history of denervation in SMA: relation to age, SMN2 copy number, and function.” Annals of neurology 57(5): 704-12; Le et al., “Temporal requirement for high SMN expression in SMA mice.” Human molecular genetics, 20(18): 3578-91; Farrar et al., “Corticomotoneuronal integrity and adaptation in spinal muscular atrophy.” Archives of neurology, 69(4): 467-73.

Patients with Types II and III SMA have a relatively stable clinical course. Furthermore, studies show that outcome differences are related to the number of SMN2 copies that enable motor neurons to adapt and compensate during the growth of the child and persist into adult life. This contrasts with SMA Type I, where motor neuron loss is profound in the early postnatal period (or may even start in the pre-natal period, especially for SMA Type I patients presenting in first three months of life). Overexpression of SMN has been shown to be well tolerated in both mice and non-human primates, and in human's high copy number of SMN2 poses no risk (as seen in Type II, III, and IV patients who have high SMN2 copy number). Increasing SMN levels in patients with SMA, e.g., Types II and III SMA presents a therapeutic option.

Therapeutic efforts in SMA, e.g., SMA types II and III thus far have focused primarily on the potential for small molecules to increase SMN levels. These include deacetylase inhibitors, such as, valproic acid, sodium butyrate, phenyl butyrate, and trichostatin A. These agents activate the SMN2 promoter, resulting in increased full-length SMN protein in SMA animal models, with the aim of modifying the disease phenotype towards the milder features seen in Type III SMA patients. Riessland et al., “SAHA ameliorates the SMA phenotype in two mouse models for spinal muscular atrophy.” Human molecular genetics, 19(8): 1492-506; Dayangac-Erden et al., “Carboxylic acid derivatives of histone deacetylase inhibitors induce full length SMN2 transcripts: a promising target for spinal muscular atrophy therapeutics.” Arch Med Sci, 7(2): 230-4 2011.

Clinical trials employing several of these agents, most notably phenyl butyrate, valproic acid, and hydroxyurea, have not resulted in sufficient clinical benefit. Darbar et al., “Evaluation of muscle strength and motor abilities in children with Type II and III spinal muscle atrophy treated with valproic acid.” BMC Neurol, 11: 36; www.ClinicalTrials.gov. FDA recently approved nusinersen, an antisense oligonucleotide (ASO) drug designed to increase the production of the SMN protein by modulating the splicing of the SMN2 gene, thereby compensating for the underlying genetic defect. Clinical studies have shown some modest promise in improving motor function; however, the treatment must be administered indefinitely on a quarterly basis via intrathecal injection, requires a lengthy induction period prior to effectiveness, and has safety considerations which require clinical monitoring. Accordingly, there remains a need for improved treatment of SMA, including SMA type II and III, using alternatives such as those disclosed herein.

Disclosed herein are compositions comprising AAV9 viral vectors and methods of using them to treat SMA, e.g., Type II and Type III SMA patients. In some embodiments, the methods comprise intrathecally injecting an AAV9 viral vector that has the ability to modify SMA, e.g., SMA Type II and Type III phenotypes, e.g., leading to a milder course of disease progression, stopped disease progression, and/or improved functional development.

### SUMMARY

The present disclosure provides compositions and methods to treat SMA, e.g., Type II or Type III SMA. Recombinant viral vectors, for example the scAAV expressing an SMN transgene disclosed herein, may provide a therapeutic method for increasing SMN levels. Since the SMN transgene is small, it can be efficiently packaged with an scAAV, allowing for lower viral titers compared with prototypical single-stranded AAV viral vectors. However, Types II and III SMA patients are often diagnosed at a later age, where they may potentially be too large to receive a safe and effective weight-based intravenous dosage of rAAV. Thus, intrathecal administration, where the AAV viral vector is delivered past the blood-brain barrier directly to the cerebrospinal fluid, may provide a safe and efficient alternative way to transfer lower viral titers.

The present disclosure provides a method of treating SMA, e.g., Type II or Type III spinal muscular atrophy (SMA) in a patient in need thereof, comprising administering intrathecally an AAV9 viral vector comprising a polynucleotide encoding a survival motor neuron (SMN) protein, wherein the viral vector is administered at a dose of about 1×10¹³ vg-5×10¹⁴ vg. In one such embodiment, the AAV9 viral vector comprises a modified AAV2 ITR, a chicken beta-actin (CB) promoter, a cytomegalovirus (CMV) immediate/early enhancer, a modified SV40 late 16S intron, a bovine growth hormone (BGH) polyadenylation signal, and an unmodified AAV2 ITR. In another embodiment, the polynucleotide encodes the SMN protein of SEQ ID NO: 2. In another embodiment, the AAV9 viral vector comprises SEQ ID NO: 1. In some embodiments, the patient is six months or older at the time of administration. In other embodiments, the patient is 24 months or younger at the time of administration, optionally between 6 months and 24 months of age. In other embodiments, the patient is 60 months or younger at the time of administration, optionally between 24 and 60 months of age. In some embodiments, the AAV9 viral vector is administered at a dose of about 5.0×10¹³ vg-3.0×10¹⁴ vg. In some embodiments, the AAV9 viral vector is administered at a dose of up to about 6.0×10¹³ vg. In some embodiments, the AAV9 viral vector is administered at a dose of about 6.0×10¹³ vg. In some embodiments, the AAV9 viral vector is administered at a dose of up to about 1.2×10¹⁴ vg. In some embodiments, the AAV9 viral vector is administered at a dose of about 1.2×10¹⁴ vg. In some embodiments, the AAV9 viral vector is administered at a dose of up to about 2.4×10¹⁴ vg. In some embodiments, the AAV9 viral vector is administered at a dose of about 2.4×10¹⁴ vg.

In some embodiments, the AAV9 viral vector is administered in a unit dose comprising about 1.0×10¹³ vg-9.9×10¹⁴ vg. In some embodiments, the AAV9 viral vector is administered in a unit dose comprising about 1.0×10¹³ vg-5.0×10¹⁴ vg. In some embodiments, the AAV9 viral vector is administered in a unit dose comprising about 5.0×10¹³ vg-3.0×10¹⁴ vg. In some embodiments, the AAV9 viral vector is administered in a unit dose comprising about 6.0×10¹³ vg. In some embodiments, the AAV9 viral vector is administered in a unit dose comprising about 1.2×10¹⁴ vg. In some embodiments, the AAV9 viral vector is administered in a unit dose comprising about 2.4×10¹⁴ vg.

In some embodiments, the patient comprises bi-allelic SMN1 null mutations or inactivating deletions, optionally wherein the mutations comprise deletion of exon seven of SMN1. In some embodiments, the patient has three copies of SMN2. In some embodiments, the patient does not have a c.859G>C substitution in exon 7 on at least one copy of the SMN2 gene. In some embodiments, the patient in need thereof is determined by one or more genomic tests. In some embodiments, patient shows onset of disease before about 12 months of age. In some embodiments, the patient has the ability to sit unassisted for about 10 or more seconds but cannot stand or walk at the time of administration. In some embodiments, the patient has the ability to sit unassisted at the time of administration, e.g., as defined by the World Health Organization Multicentre Growth Reference Study (WHO-MGRS) criteria. In some embodiments, the patient has the ability to stand without support for at least about three seconds after administration, e.g., as defined by the Bayley Scales of Infant and Toddler Development®, e.g., as assessed about 1-24 months, e.g., 12 months, after administration. In some embodiments, the patient has the ability to walk without assistance after administration, e.g., as defined by the Bayley Scales of Infant and Toddler Development®, e.g., as assessed about 1-24 months, e.g., about 12 months after administration. In some embodiments, the patient has the ability to take at least five steps independently after administration, e.g., as defined by the Bayley Scales of Infant and Toddler Development®, as assessed about 1-24 months, e.g., about 12 months after administration. In some embodiments, the patient shows a change after treatment from a baseline measurement at time of treatment, e.g., as defined by the Bayley Scales of Infant and Toddler Development®, as assessed about 1-24 months, e.g., about 12 months after administration.

In some embodiments, the patient does not have severe scoliosis after administration, e.g., 50° curvature of spine evident on X-ray examination, as assessed about 1-24 months, e.g., about 12 months after administration. In some embodiments, the patient is not contraindicated for spinal tap procedure or administration of intrathecal therapy. In some embodiments, the patient has not previously had a scoliosis repair surgery or procedure, and optionally wherein the patient does not have a scoliosis repair surgery or procedure within 6 months to 3 years, e.g., within 1 year after administration. In some embodiments, the patient does not need the use of invasive ventilatory support before and/or after administration. In some embodiments, the patient does not have a history of standing or walking independently prior to administration. In some embodiments, the patient does not use a gastric feeding tube before and/or after administration. In some embodiments, the patient does not have an active viral infection at the time of treatment (including human immunodeficiency virus (HIV) or serology positive for hepatitis B or C or Zika virus). In some embodiments, the patient has not had a severe non-pulmonary/respiratory tract infection (e.g., pyelonephritis or meningitis) within four weeks prior to administration. In some embodiments, the patient does not have concomitant illness, e.g., major renal or hepatic impairment, known seizure disorder, diabetes mellitus, idiopathic hypocalciuria or symptomatic cardiomyopathy prior to administration. In some embodiments, the patient does not have a history of bacterial meningitis or brain or spinal cord disease prior to administration. In some embodiments, the patient does not have a known allergy or hypersensitivity to prednisolone or other glucocorticosteroids or excipients prior to administration. In some embodiments, the patient does not have a known allergy or hypersensitivity to iodine or iodine-containing products prior to administration. In some embodiments, the patient is not taking drugs to treat myopathy or neuropathy. In some embodiments, the patient is not receiving immunosuppressive therapy, plasmapheresis, immunomodulators such as adalimumab, within 3 months prior to administration.

In some embodiments, the patient has anti-AAV9 antibody titers at or below 1:25, 1:50, 1:75, or 1:100, e.g., as determined by an ELISA binding immunoassay, prior to administration. In some embodiments, the patient has one or more of gamma-glutamyl transferase levels less than about 3 times upper limit of normal, bilirubin levels less than about 3.0 mg/dL, creatinine levels less than about 1.0 mg/dL, Hgb levels between about 8-18 g/dL, and/or white blood cell counts of less than about 20000 per mm³ prior to administration. In some embodiments, the patient has not received an investigational or approved compound product or therapy with the intent to treat SMA prior to administration. In some embodiments, wherein the AAV9 viral vector is administered together with a contrast medium, optionally wherein the contrast medium comprises iohexol. In some embodiments, the volume of contrast medium administered is about 1.0-2.0 mL, e.g., about 1.5 mL, optionally wherein the contrast medium is mixed with the AAV9 viral vector prior to administration, e.g., less than 24 h, less than 12 h, less than 6 h, less than 5 h, less than 4 h, less than 3 h, less than 2 h, less than 1 h, less than 30 minutes or immediately prior to administration. In some embodiments, the contrast medium and the AAV9 viral vector are administered sequentially, for example, wherein a contrast medium is administered (e.g., intrathecally) first and the AAV9 viral vector is administered (e.g., intrathecally) subsequent to administration of the contrast medium. In some embodiments, the contrast medium and the AAV9 viral vector are administered sequentially, for example, wherein a AAV9 viral vector is administered (e.g., intrathecally) first and the contrast medium is administered (e.g., intrathecally) subsequent to the administration of the AAV9 viral vector. In embodiments where the AAV9 viral vector and contrast medium are administered sequentially, the administration of the AAV9 viral vector and the contrast medium are administered within 2 hours, within 1 hour, within 45 minutes, within 30 minutes, within 15 minutes, within 10 minutes or within 5 minutes of each other. In some embodiments, wherein the total volume of AAV9 viral vector and contrast medium administered to the patient does not exceed about 10 mL, about 9 mL, or about 8 mL. In some embodiments, the method further comprises sedation or anesthesia. In some embodiments, the patient is placed in the Trendelenburg position during and/or after administration of the AAV9 viral vector. In some embodiments, the patient is placed tilted head-down at about 30° for about 10-60 minutes, e.g., about 15 minutes, after administration of the AAV9 viral vector.

In some embodiments, the patient is administered an oral steroid at least about 1-48 hours, e.g., about 24 hours prior to administering the AAV9 viral vector. In some embodiments, the patient is administered an oral steroid for at least about 10-60 days, e.g., about 30 days, after administering the viral vector. In some embodiments, the oral steroid is administered once daily. In some embodiments, the oral steroid is administered twice daily. In some embodiments, the patient is monitored for levels of ALT and/or AST after the administration of the viral vector, and wherein the oral steroid continues to be administered after 30 days until AST and/or ALT levels are below twice the upper limit of normal or below about 120 IU/L. In some embodiments, the patient is monitored for levels of T cell response after the administration of the AAV9 viral vector, and wherein the oral steroid continues to be administered after 30 days until T cell response in a sample from the patient, e.g., a blood sample, falls below 100 spot forming cells (SFC) per 10⁶ peripheral blood mononuclear cells (PBMCs).

In some embodiments, the oral steroid is administered at a dose of about 1 mg/kg.

In some embodiments, the oral steroid is tapered after AST and ALT are below twice the upper limit of normal or below about 120 IU/L. In some embodiments, the tapering comprises stepped increments to about 0.5 mg/kg/day for 2 weeks followed by about 0.25 mg/kg/day for 2 more weeks. In some embodiments, the oral steroid is administered for 30 days at a dose of about 1 mg/kg and then tapering down to 0.5 mg/kg/day for 2 weeks followed by 0.25 mg/kg/day for 2 more weeks. In some embodiments, the oral steroid is prednisolone or an equivalent.

In some embodiments, the treatment efficacy is determined using the Bayley Scales of Infant and Toddler Development® scale and/or the Hammersmith Functional Motor Scale-Expanded (HFMSE). In some embodiments, the method further comprises administering a second therapeutic agent to the patient concomitantly or consecutively with the administration of the AAV9 viral vector. In some such embodiments, the second therapeutic agent comprises a muscle enhancer or neuroprotector. In other such embodiments, the second therapeutic agent comprises an antisense oligonucleotide or antisense oligonucleotides targeting SMN1 and/or SMN2. In some embodiments, the second therapeutic agent comprises nusinersen and/or stamulumab. In some embodiments, wherein the amount of AAV9 viral vector genome is measured using ddPCR. In some embodiments, the patient has anti-AAV9 antibody titers at or above 1:25, 1:50, 1:75, or 1:100, e.g., as determined by an ELISA binding immunoassay, after administration and is monitored for about 1-8 weeks or until titers decrease to below 1:25, 1:50, 1:75, or 1:100. In some embodiments, the patient has anti-AAV9 antibody titers at or above 1:25, 1:50, 1:75, or 1:100, e.g., as determined by an ELISA binding immunoassay, after administration and is administered a steroid, e.g., prednisolone, until titers decrease to below 1:25, 1:50, 1:75, or 1:100. In some embodiments, the patient has platelet counts above about 67,000 cells/ml prior to administration or above about 100,000 cells/ml, or above about 150,000, cells/ml. In some embodiments, the patient has platelet counts below about 67,000 cells/ml after administration, or below about 100,000 cells/ml, or below about 150,000, cells/ml, and is monitored for about 1-8 weeks or until platelet counts increase to about 67,000 cells/ml, or above about 100,000 cells/ml, or above about 150,000, cells/ml. In some embodiments, the patient has platelet counts below about 67,000 cells/ml after administration and is treated with a platelet transfusion. In some embodiments, the patient has normal hepatic function prior to administration of the AAV9 viral vector. In some embodiments, the patient has hepatic transaminase levels less than about 8-40 U/L prior to administration.

In some embodiments, the hepatic transaminase is selected from AST, ALT, and a combination thereof. In some embodiments, the AAV9 viral vector is in a pharmaceutical formulation suitable for intrathecal administration.

The present disclosure also provides a use of an AAV9 viral vector in the treatment of SMA, e.g., Type II or Type III spinal muscular atrophy (SMA) according to the methods described herein.

The present disclosure provides a pharmaceutical composition comprising an AAV9 viral vector and a pharmaceutically acceptable carrier suitable for intrathecal administration, wherein the AAV9 viral vector comprises a modified AAV2 ITR, a chicken beta-actin (CB) promoter, a cytomegalovirus (CMV) immediate/early enhancer, a modified SV40 late 16S intron, a bovine growth hormone (BGH) polyadenylation signal, and an unmodified AAV2 ITR. In some embodiments, the polynucleotide encodes the SMN protein of SEQ ID NO: 2. In some embodiments, the AAV9 viral vector comprises SEQ ID NO: 1. In some embodiments, the pharmaceutical composition further comprises a contrast agent. In some embodiments, the contrast agent is present in an amount of about 1.0-2.0 mL, e.g., about 1.5 mL.

In some embodiments, the total volume of AAV9 viral vector and contrast medium does not exceed about 10 mL, about 9 mL, or about 8 mL. In some embodiments, the pharmaceutical composition further comprises an additional therapeutic agent. In some embodiments, the pharmaceutical composition is for use in any of the methods of treatment described herein.

In some embodiments, the pharmaceutical composition is a unit dose comprising about 1.0×10¹³ vg-9.9×10¹⁴ vg. In some embodiments, the pharmaceutical composition is a unit dose comprising about 1.0×10¹³ vg-5.0×10¹⁴ vg. In some embodiments, the pharmaceutical composition is a unit dose comprising about 5.0×10¹³ vg-3.0×10¹⁴ vg.

In some embodiments, the pharmaceutical composition is a unit dose comprising about 6.0×10¹³ vg. In some embodiments, the pharmaceutical composition is a unit dose comprising about 1.2×10¹⁴ vg. In some embodiments, the pharmaceutical composition is a unit dose comprising about 2.4×10¹⁴ vg.

In some embodiments, the pharmaceutical composition comprises at least one of the following: (a) about pH 7.7-8.3, (b) about 390-430 mOsm/kg, (c) less than about 600 particles that are ≥25 μm in size per container, (d) less than about 6000 particles that are ≥10 μm in size per container, (e) about 1.7×10¹³-5.3×10¹³ vg/mL genomic titer, (f) infectious titer of about 3.9×10⁸-8.4×10¹⁰ IU per 1.0×10¹³ vg, (g) total protein of about 100-300 μg per 1.0×10¹³ vg, (h) Pluronic F-68 content of about 20-80 ppm, (i) relative potency of about 70-130%, (j) median survival in a SMNΔ7 mouse model greater than or equal to 24 days at a dose of 7.5×10¹³ vg/kg, (k) less than about 5% empty capsid, (l) and a total purity of greater than or equal to about 95%, and (m) less than or equal to about 0.13 EU/mL Endotoxin.

In some embodiments, the pharmaceutical composition comprises at least one of the following conditions: (a) less than about 0.09 ng of benzonase per 1.0×10¹³ vg, (b) less than about 30 μg/g (ppm) of cesium, (c) about 20-80 ppm of Poloxamer 188, (d) less than about 0.22 ng of BSA per 1.0×10¹³ vg, (e) less than about 6.8×10⁵ pg of residual plasmid DNA per 1.0×10¹³ vg, (f) less than about 1.1×10⁵ pg of residual hcDNA per 1.0×10¹³ vg, (g) less than about 4 ng of rHCP per 1.0×10¹³ vg, (h) about pH 7.7-8.3, (i) about 390-430 mOsm/kg, (j) less than about 600 particles that are ≥25 μm in size per container, (k) less than about 6000 particles that are ≥10 μm in size per container, (l) about 1.7×10¹³-5.3×10¹³ vg/mL genomic titer, (m) infectious titer of about 3.9×10⁸-8.4×10¹⁰ IU per 1.0×10¹³ vg, (n) total protein of about 100-300 μg per 1.0×10¹³ vg, (o) relative potency of about 70-130%, and (p) less than about 5% empty capsid.

In some embodiments, the methods or use of compositions described herein results in an improved score on the Hammersmith Functional Motor Scale-Expanded, relative to pre-administration scores. In some embodiments, the methods or use of compositions described herein results in an improved score on the Bayley Scales of Infant and Toddler Development®, Third Edition (Bayley®-III), relative to pre-administration scores.

### BRIEF DESCRIPTION OF THE DRAWINGS

FIG. 1 shows body mass of treated and control mice following AAV administration.

FIG. 2 shows the initial study design of the Phase I, open label single dose administration study of infants and children with Type II or Type III SMA. Patients receive AVXS-101 in a dose comparison safety study.

FIG. 3 shows a waterfall plot of change from baseline, ranked highest to lowest, for Hammersmith Functional Motor Scale Expanded (HFMSE) in SMA Type 2 patients receiving Dose A (6.0×10¹³ vg; noted by diamond) or Dose B (1.2×10¹⁴ vg) intrathecal AVXS-101 assessed after 24 months of age. Results for patients aged between six months and two years at time of infusion are depicted by grey bars; black bars indicate ages between 2 and 5 years at time of infusion.

FIG. 4 shows the HFMSE scores of individual patients with SMA Type 2.

FIG. 5 shows the response to AVXS-101 treatment, as measured by the HFMSE, in patients aged between six months and five years at the time of treatment.

FIG. 6 shows the response to AVXS-101 treatment, as measured by the HFMSE, in patients aged between two years and five years at the time of treatment who received a dose of 1.2×10¹⁴ vg.

FIG. 7 shows a spaghetti plot of change from baseline in HFMSE Scores up to Month 12 for the 24 months and <60 months age group (Primary PNCR Analysis)—ITT Set.

FIG. 8 shows a spaghetti plot of change from baseline in HFMSE Scores up to Month 12 for the 24 months and <60 months age group (Sensitivity PNCR Analysis)—ITT Set.

FIG. 9 shows a spaghetti plot of change from baseline in fine motor score as determined by Bayley Scales® at each post-baseline visit up to 12 months for patients <24 months of age at time of dosing—ITT Set.

FIG. 10 shows a spaghetti plot of change from baseline in gross motor score as determined by Bayley Scales® at each post-baseline visit up to 12 months for patients <24 months of age at time of dosing—ITT Set.

FIG. 11 shows a spaghetti plot of change from baseline in fine motor score as determined by Bayley Scales® at each post-baseline visit up to 12 months for patients ≥24 and <60 months of age at time of dosing—ITT Set.

FIG. 12 shows a spaghetti plot of change from baseline in gross motor score as determined by Bayley Scales® at each post-baseline visit up to 12 months for patients ≥24 and <60 months of age at time of dosing—ITT Set.

FIG. 13 shows a spaghetti plot of change from baseline in HFMSE at each post-baseline at each visit for patients <24 months of age at time of dosing who continue in the study past 24 months of age—ITT Set.

### DETAILED DESCRIPTION

In order to better understand the disclosure, certain exemplary embodiments are discussed herein. In addition, certain terms are discussed to aid in the understanding.

In some embodiments, by “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

In some embodiments, by an “AAV vector” is meant a vector derived from an adeno-associated virus serotype, including without limitation, AAV-1, AAV-2, AAV-3, AAV-4, AAV-5, AAV-6, AAV-7, AAV-8 and AAV-9. AAV vectors can have one or more of the AAV wild-type genes deleted in whole or part, e.g., the rep and/or cap genes, but retain functional flanking ITR sequences. Functional ITR sequences are necessary for the rescue, replication and packaging of the AAV virion. Thus, an AAV vector is defined herein to include at least those sequences that in cis provide for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. In one embodiment, the vector is an AAV-9 vector, with AAV-2 derived ITRs. Also, by an “AAV vector” is meant the protein shell or capsid, which provides an efficient vehicle for delivery of vector nucleic acid to the nucleus of target cells.

In some embodiments, by “scAAV” is meant a self-complementary adeno-associated virus (scAAV), which is a viral vector engineered from the naturally occurring adeno-associated virus (AAV) for use in gene therapy. scAAV is termed “self-complementary” because the coding region has been designed to form an intramolecular double-stranded DNA template.

In some embodiments, “recombinant virus” is meant a virus that has been genetically altered, e.g., by the addition or insertion of a heterologous nucleic acid construct into the particle. “Recombinant” may abbreviated “r”, e.g., rAAV may refer to recombinant AAV. The term “AAV” as used herein is intended to encompass “recombinant AAV” or “rAAV.”

In some embodiments, by “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion and both strands are equally infectious.

In some embodiments, the terms “recombinant AAV virion,” “rAAV virion,” “AAV vector particle,” “full capsids,” and “full particles” are defined herein as an infectious, replication-defective virus including an AAV protein shell, encapsidating a heterologous nucleotide sequence of interest which is flanked on both sides by AAV ITRs. A rAAV virion is produced in a suitable host cell which has had sequences specifying an AAV vector, AAV helper functions and accessory functions introduced therein. In this manner, the host cell is rendered capable of encoding AAV polypeptides that provide for packaging the AAV vector (containing a recombinant nucleotide sequence of interest) into infectious recombinant virion particles for subsequent gene delivery.

Unless otherwise defined, all technical and scientific terms used herein have the same meaning as commonly understood by one of ordinary skill in the art to which this disclosure belongs. All references cited herein are incorporated by reference in their entireties. To the extent terms or discussion in references conflict with this disclosure, the latter shall control.

As used herein, the singular forms of a word also include the plural form of the word, unless the context clearly dictates otherwise; as examples, the terms “a,” “an,” and “the” are understood to be singular or plural. By way of example, “an element” means one or more element. The term “or” shall mean “and/or” unless the specific context indicates otherwise.

The term “comprising,” or variations such as “comprises,” will be understood to imply the inclusion of a stated element, integer or step, or group of elements, integers or steps, but not the exclusion of any other element, integer or step, or group of elements, integers or steps. Throughout the specification the word “consisting of,” or variations such as “consists of,” will be understood to imply the inclusion of a stated element, integer or step, or group of elements, integers or steps, and the exclusion of any other element, integer or step, or group of elements, integers or steps. Throughout the specification the word “consisting essentially of,” or variations such as “consists essentially of,” will be understood to imply the inclusion of a stated element, integer or step, or group of elements, integers or steps, and any other element, integer or step, or group of elements, integers or steps that do not materially affect the basic and novel characteristics of the disclosure and/or claim.

About can be understood as within +/−10%, e.g., +/−10%, 9%, 8%, 7%, 6%, 5%, 4%, 3%, 2%, 1%, 0.5%, 0.1%, 0.05%, or 0.01% of the stated value. When used in reference to a percentage value, “about” can be understood as within ±1% (e.g., “about 5%” can be understood as within 4%-6%) or ±0.5% (e.g., “about 5%” can be understood as within 4.5%-5.5%). Unless otherwise clear from the context, all numerical values provided herein are modified by the term “about.” All ranges used herein encompass the endpoints.

rAAV Viral Vector

In one aspect, disclosed herein are rAAV genomes. In some embodiments, an rAAV genome comprises one or more AAV ITRs flanking a polynucleotide encoding an SMN polypeptide. In some embodiments, the polynucleotide is operatively linked to transcriptional control DNA elements, e.g., a promoter DNA, one or more enhancer DNAs, and/or a polyadenylation signal sequence DNA that are functional in target cells to form a gene cassette. The gene cassette may also include intron sequences to facilitate processing of an RNA transcript when expressed in mammalian cells.

In some embodiments, the rAAV genomes disclosed herein lack AAV rep and cap DNA. AAV DNA in the rAAV genomes (e.g., ITRs) may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10 and AAV-11. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC 001401 and Srivastava et al., Virol., 45: 555-564 {1983): the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004).

As used herein, the “pSMN” vector plasmid comprises a polynucleotide encoding an SMN protein, i.e, a SMN cDNA expression cassette, wherein the cassette is flanked by adeno-associated virus inverted terminal repeat (ITR) sequences, e.g., “left” and “right” of the polynucleotide encoding the SMN gene. In some embodiments, the polynucleotide encoding SMN is a human SMN sequence, e.g., a naturally occurring human SMN sequence or isoforms, variants, or mutants thereof. In some embodiments, the ITR sequences are native, variant, or modified AAV ITR sequences. In some embodiments, at least one ITR sequence is a native, variant, or modified AAV2 ITR sequence. In some embodiments, the two ITR sequences are both native, variant, or modified AAV2 ITR sequences. In some embodiments, the “left” ITR is a modified AAV2 ITR sequence that allows for the production of self-complementary genomes, and the “right” ITR is a native AAV2 ITR sequence. In some embodiments, the “right” ITR is a modified AAV2 ITR sequence that allows for the production of self-complementary genomes, and the “left” ITR is a native AAV2 ITR sequence. In some embodiments, the pSMN plasmid further comprises a CMV enhancer/chicken beta-actin (“CB”) promoter. In some embodiments, the pSMN plasmid further comprises a a Simian Virus 40 (SV40) intron. In some embodiments, the pSMN plasmid further comprises a bovine growth hormone (BGH) polyadenylation (polyA) termination signal. Exemplary sequences that may be used for one or more of the components discussed above are shown in Table 1 below. In some embodiments, all of the sequences shown in Table 1 below are used. In some embodiments, “AVXS-101,” is a non-limiting example of a vector construct using all the sequences in Table 1 and falling within the scope of the term pSMN. Embodiments of these vectors and methods of preparing and purifying them are provided, e.g., in PCT/US2018/058744, which is incorporated herein by reference in its entirety.

In some embodiments, a pSMN vector may comprise a SMN cDNA expression cassette, a modified AAV2 ITR, a chicken beta-actin (CB) promoter, a cytomegalovirus (CMV) immediate/early enhancer, a modified SV40 late 16s intron, a bovine growth hormone (BGH) polyadenylation signal, and an unmodified AAV2 ITR. The modified and unmodified ITRs may come in either orientation (i.e., 5′ or 3′) relative to the SMN cDNA expression cassette.

TABLE 1
AVXS-101 Vector Construct DNA Sequence Summary Component (all
nt start and stop positions are in relation to SEQ ID NO: 1).

| | | | | | Non-limiting |
|---|---|---|---|---|---|
| | Start | Stop | Size | | description of |
| | Position | Position | (nt) | Description | potential benefits |
| “Left” Mutated AAV2 | 1 | 106 | 106 | Modification | Without being |
| ITR | | | | to the “left” | limited by theory, |
| | | | | ITR by | this mutated ITR |
| | | | | deleting the | may allow for a |
| | | | | terminal | second-generation |
| | | | | resolution | self- |
| | | | | site to allow | complementary |
| | | | | hairpin | vector to maximize |
| | | | | formation of | vector potency, |
| | | | | genome | allowing lower |
| | | | | | systemic doses |
| CMV Enhancer/CB | 153 | 432 | 280 | Portion of | Without being |
| Promoter | | | | the CMV | limited by theory, |
| | | | | immediate/ | this may allow for |
| | | | | early | constitutive high- |
| | | | | enhancer | level SMN |
| | 439 | 704 | 266 | CB core | expression |
| | | | | promoter | |
| SV40 Intron | 774 | 870 | 97 | Intron from | Without being |
| | | | | the SV40 (to | limited by theory, |
| | | | | enhance | this may allow for |
| | | | | accumulation | increased gene |
| | | | | of steady | expression |
| | | | | level of | |
| | | | | mRNA for | |
| | | | | translation) | |
| Human SMN cDNA | 1003 | 1887 | 885 | Modified | Without being |
| | | | | from | limited by theory, |
| | | | | Genbank | this may allow the |
| | | | | Accession | for expression of a |
| | | | | #NM_017411 | full-length SMN |
| | | | | | protein |
| BGH Poly A | 1973 | 2204 | 232 | BGH Poly A | Without being |
| Termination Signal | | | | signal | limited by theory, |
| | | | | | this may provide a |
| | | | | | Poly A of the SMN |
| | | | | | mRNA |
| | | | | | (transcription |
| | | | | | termination signal) |
| | | | | | for high-level, |
| | | | | | efficient gene |
| | | | | | expression |
| “Right” AAV2 ITR | 2217 | 2359 | 143 | Unmodified | Without being |
| | | | | AAV2 ITR | limited by theory, |
| | | | | | this AAV2 ITR in |
| | | | | | cis may provide for |
| | | | | | both viral DNA |
| | | | | | replication and |
| | | | | | packaging of the |
| | | | | | AAV vector |
| | | | | | genome |

In some embodiments, the vector construct sequence is encapsidated, e.g., into AAV9 virions. In these embodiments, encapsidation is in a non-replicating, recombinant AAV9 capsid capable of delivering a stable, function transgene, e.g. a fully functional human SMN transgene. In some embodiments, the capsid is comprised of 60 viral proteins (VP1, VP2, VP3), e.g., in a ratio of 1:1:10 produced by alternate splicing such that VP2 and VP3 are two truncated forms of VP1, all with common C-terminal sequences. In some embodiments, the product of the manufacturing process, e.g., a drug product, may comprise a non-replicating, recombinant AAV9 capsid to deliver a stable, fully functional human SMN transgene. In some embodiments, the capsid is comprised of 60 viral proteins (VP1, VP2, VP3) in a ratio of 1:1:10 produced by alternate splicing such that VP2 and VP3 are two truncated forms of VP1, all with common C-terminal sequences. Embodiments of these vector constructs and methods of preparing and purifying them are provided, e.g., in PCT/US2018/058744, which is incorporated herein by reference in its entirety.

In various embodiments, the DNA sequence of a pSMN vector construct, e.g., AVXS-101 vector construct, comprises SEQ ID NO: 1:

| (SEQ ID NO: 1) | |
|---|---|
| ctgcgcgctc gctcgctcac tgaggccgcc cgggcaaagc ccgggcgtcg | 50 |
| ggcgaccttt ggtcgcccgg cctcagtgag cgagcgagcg cgcagagagg | 100 |
| gagtggaatt cacgcgtgga tctgaattca attcacgcgt ggtacctctg | 150 |
| gtcgttacat aacttacggt aaatggcccg cctggctgac cgcccaacga | 200 |
| cccccgccca ttgacgtcaa taatgacgta tgttcccata gtaacgccaa | 250 |
| tagggacttt ccattgacgt caatgggtgg agtatttacg gtaaactgcc | 300 |
| cacttggcag tacatcaagt gtatcatatg ccaagtacgc cccctattga | 350 |
| cgtcaatgac ggtaaatggc ccgcctggca ttatgcccag tacatgacct | 400 |
| tatgggactt tcctacttgg cagtacatct actcgaggcc acgttctgct | 450 |
| tcactctccc catctccccc ccctccccac ccccaatttt gtatttattt | 500 |
| attttttaat tattttgtgc agcgatgggg gcgggggggg ggggggggcg | 550 |
| cgcgccaggc ggggcggggc ggggcgaggg gcggggcggg gcgaggcgga | 600 |
| gaggtgcggc ggcagccaat cagagcggcg cgctccgaaa gtttcctttt | 650 |
| atggcgaggc ggcggcggcg gcggccctat aaaaagcgaa gcgcgcggcg | 700 |
| ggcgggagcg ggatcagcca ccgcggtggc ggcctagagt cgacgaggaa | 750 |
| ctgaaaaacc agaaagttaa ctggtaagtt tagtcttttt gtcttttatt | 800 |
| tcaggtcccg gatccggtgg tggtgcaaat caaagaactg ctcctcagtg | 850 |
| gatgttgcct ttacttctag gcctgtacgg aagtgttact tctgctctaa | 900 |
| aagctgcgga attgtacccg cggccgatcc accggtccgg aattcccggg | 950 |
| atatcgtcga cccacgcgtc cgggccccac gctgcgcacc cgcgggtttg | 1000 |
| ctatggcgat gagcagcggc ggcagtggtg gcggcgtccc ggagcaggag | 1050 |
| gattccgtgc tgttccggcg cggcacaggc cagagcgatg attctgacat | 1100 |
| ttgggatgat acagcactga taaaagcata tgataaagct gtggcttcat | 1150 |
| ttaagcatgc tctaaagaat ggtgacattt gtgaaacttc gggtaaacca | 1200 |
| aaaaccacac ctaaaagaaa acctgctaag aagaataaaa gccaaaagaa | 1250 |
| gaatactgca gcttccttac aacagtggaa agttggggac aaatgttctg | 1300 |
| ccatttggtc agaagacggt tgcatttacc cagctaccat tgcttcaatt | 1350 |
| gattttaaga gagaaacctg tgttgtggtt tacactggat atggaaatag | 1400 |
| agaggagcaa aatctgtccg atctactttc cccaatctgt gaagtagcta | 1450 |
| ataatataga acagaatgct caagagaatg aaaatgaaag ccaagtttca | 1500 |
| acagatgaaa gtgagaactc caggtctcct ggaaataaat cagataacat | 1550 |
| caagcccaaa tctgctccat ggaactcttt tctccctcca ccacccccca | 1600 |
| tgccagggcc aagactggga ccaggaaagc caggtctaaa attcaatggc | 1650 |
| ccaccaccgc caccgccacc accaccaccc cacttactat catgctggct | 1700 |
| gcctccattt ccttctggac caccaataat tcccccacca cctcccatat | 1750 |
| gtccagattc tcttgatgat gctgatgctt tgggaagtat gttaatttca | 1800 |
| tggtacatga gtggctatca tactggctat tatatgggtt ttagacaaaa | 1850 |
| tcaaaaagaa ggaaggtgct cacattcctt aaattaagga gaaatgctgg | 1900 |
| catagagcag cactaaatga caccactaaa gaaacgatca gacagatcta | 1950 |
| gaaagcttat cgataccgtc gactagagct cgctgatcag cctcgactgt | 2000 |
| gccttctagt tgccagccat ctgttgtttg cccctccccc gtgccttcct | 2050 |
| tgaccctgga aggtgccact cccactgtcc tttcctaata aaatgaggaa | 2100 |
| attgcatcgc attgtctgag taggtgtcat tctattctgg ggggtggggt | 2150 |
| ggggcaggac agcaaggggg aggattggga agacaatagc aggcatgctg | 2200 |
| gggagagatc gatctgagga acccctagtg atggagttgg ccactccctc | 2250 |
| tctgcgcgct cgctcgctca ctgaggccgg gcgaccaaag gtcgcccgac | 2300 |
| gcccgggctt tgcccgggcg gcctcagtga gcgagcgagc gcgcagagag | 2350 |
| ggagtggcc. | 2359 |

In some embodiments, the amino acid sequence of the SMN protein encoded by the pSMN plasmid, e.g., AVX101, comprises:

(SEQ ID NO: 2)
MAMSSGGSGGGVPEQEDSVLFRRGTGQSDDSDIWDDTALIKAYDKAVAS
FKHALKNGDICETSGKPKTTPKRKPAKKNKSQKKNTAASLQQWKVGDKC
SAIWSEDGCIYPATIASIDFKRETCVVVYTGYGNREEQNLSDLLSPICE
VANNIEQNAQENENESQVSTDESENSRSPGNKSDNIKPKSAPWNSFLPP
PPPMPGPRLGPGKPGLKFNGPPPPPPPPPPHLLSCWLPPFPSGPPIIPP
PPPICPDSLDDADALGSMLISWYMSGYHTGYYMGFRQNQKEGRCSHSLN.

In some embodiments, AAV capsid proteins VP1, VP2, VP3 are derived from the same transcript. These have alternative start sites but share a carboxy terminus. Below, VP1 specific amino acid sequences are shown in black and are bolded. Amino acid sequences common to VP1 and VP2 are underlined and in italics. Amino acids common to all three capsid proteins are bolded and in italics.

| (SEQ ID NO: 3) | |
|---|---|
| 1 | MAADGYLPDW LEDNLSEGIR EWWALKPGAP QPKANQQHQD NARGLVLPGY KYLGPGNGLD |
| 61 | KGEPVNAADA AALEHDKAYD QQLKAGDNPY LKYNHADAEF QERLKEDTSF GGNLGRAVFQ |
| 121 | AKKRLLEPLG LVEEAAKTAP GKKRPVEQSP QEPDSSAGIG KSGAQPAKKR LNFGQTGDTE |
| 181 | SVPDPQPIGE PPAAPSGVGS LT [symbol] [symbol] [symbol] [symbol] |
| 241 | [symbol] [symbol] [symbol] [symbol] [symbol] [symbol] |
| 301 | [symbol] [symbol] [symbol] [symbol] [symbol] [symbol] |
| 361 | [symbol] [symbol] [symbol] [symbol] [symbol] [symbol] |
| 421 | [symbol] [symbol] [symbol] [symbol] [symbol] [symbol] |
| 481 | [symbol] [symbol] [symbol] [symbol] [symbol] [symbol] |
| 541 | [symbol] [symbol] [symbol] [symbol] [symbol] [symbol] |
| 601 | [symbol] [symbol] [symbol] [symbol] [symbol] [symbol] |
| 661 | [symbol] [symbol] [symbol] [symbol] [symbol] [symbol] |
| 721 | [symbol] [symbol] . |

In one embodiment, the AAV capsid proteins are derived from a transcript encoding the amino acid sequence set forth in SEQ ID NO: 3.

In various embodiments, disclosed herein are DNA plasmids comprising rAAV genomes. The DNA plasmids are transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV particles, in which an AAV genome to be packaged, rep and cap genes, and helper virus functions are provided to a cell are available in the art. In some embodiments, production of rAAV involves the following components present within a single cell (denoted herein as a packaging cell): a rAAV genome, AAV rep and cap genes separate from (i.e., not in) the rAAV genome, and helper virus functions. Production of pseudotyped rAAV is disclosed in, for example, WO 01/83692 which is incorporated by reference herein in its entirety. In various embodiments, AAV capsid proteins may be modified to enhance delivery of the recombinant vector. Modifications to capsid proteins are generally known in the art. See, for example, US 2005/0053922 and US 2009/0202490, the disclosures of which are incorporated by reference herein in their entirety.

General principles of rAAV production are reviewed in, for example, Carter, 1992, Current Opinions in Biotechnology, 1533-539; and Muzyczka, 1992, CUM Topics in Microbial. and Immunol., 158:97-129). Various approaches are described in Ratschin et al., Mol. Cell. Biol. 4:2072 (1984); Hennonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. In addition, the rAAV disclosed herein may be prepared, purified, manufactured, and/or formulated according to the disclosure of PCT/US2018/058744. The foregoing documents are hereby incorporated by reference in their entirety herein, with particular emphasis on those sections of the documents relating to rAAV preparation, purification, production, manufacturing, and formulation.

In another aspect, rAAV comprising a polynucleotide encoding an SMN protein, such as the rAAV9 discussed herein, are referred to as “rAAV SMN.” In some embodiments, the rAAV SMN genome has in sequence a first AAV2 ITR, the chicken-β actin promoter with a cytomegalovirus enhancer, an SV40 intron, a polynucleotide encoding SMN, a polyadenylation signal sequence from bovine growth hormone, and a second AAV2 ITR. In some embodiments, polynucleotide encoding SMN is a human SMN gene, e.g., set forth in or derived from GenBank Accession Number MN_000344.2, Genbank Accession #NM_017411, or any other suitable human SMN isoform. An exemplary SMN sequence comprises a sequence of:

| (SEQ ID NO: 4) | |
|---|---|
| 1 | CCACAAATGT GGGAGGGCGA TAACCACTCG TAGAAAGCGT GAGAAGTTAC TACAAGCGGT |
| 61 | CCTCCCGGCC ACCGTACTGT TCCGCTCCCA GAAGCCCCGG GCGGCGGAAG TCGTCACTCT |
| 121 | TAAGAAGGGA CGGGGCCCCA CGCTGCGCAC CCGCGGGTTT GCTATGGCGA TGAGCAGCGG |
| 181 | CGGCAGTGGT GGCGGCGTCC CGGAGCAGGA GGATTCCGTG CTGTTCCGGC GCGGCACAGG |
| 241 | CCAGAGCGAT GATTCTGACA TTTGGGATGA TACAGCACTG ATAAAAGCAT ATGATAAAGC |
| 301 | TGTGGCTTCA TTTAAGCATG CTCTAAAGAA TGGTGACATT TGTGAAACTT CGGGTAAACC |
| 361 | AAAAACCACA CCTAAAAGAA AACCTGCTAA GAAGAATAAA AGCCAAAAGA AGAATACTGC |
| 421 | AGCTTCCTTA CAACAGTGGA AAGTTGGGGA CAAATGTTCT GCCATTTGGT CAGAAGACGG |
| 481 | TTGCATTTAC CCAGCTACCA TTGCTTCAAT TGATTTTAAG AGAGAAACCT GTGTTGTGGT |
| 541 | TTACACTGGA TATGGAAATA GAGAGGAGCA AAATCTGTCC GATCTACTTT CCCCAATCTG |
| 601 | TGAAGTAGCT AATAATATAG AACAGAATGC TCAAGAGAAT GAAAATGAAA GCCAAGTTTC |
| 661 | AACAGATGAA AGTGAGAACT CCAGGTCTCC TGGAAATAAA TCAGATAACA TCAAGCCCAA |
| 721 | ATCTGCTCCA TGGAACTCTT TTCTCCCTCC ACCACCCCCC ATGCCAGGGC CAAGACTGGG |
| 781 | ACCAGGAAAG CCAGGTCTAA AATTCAATGG CCCACCACCG CCACCGCCAC CACCACCACC |
| 841 | CCACTTACTA TCATGCTGGC TGCCTCCATT TCCTTCTGGA CCACCAATAA TTCCCCCACC |
| 901 | ACCTCCCATA TGTCCAGATT CTCTTGATGA TGCTGATGCT TTGGGAAGTA TGTTAATTTC |
| 961 | ATGGTACATG AGTGGCTATC ATACTGGCTA TTATATGGGT TTCAGACAAA ATCAAAAAGA |
| 1021 | AGGAAGGTGC TCACATTCCT TAAATTAAGG AGAAATGCTG GCATAGAGCA GCACTAAATG |
| 1081 | ACACCACTAA AGAAACGATC AGACAGATCT GGAATGTGAA GCGTTATAGA AGATAACTGG |
| 1141 | CCTCATTTCT TCAAAATATC AAGTGTTGGG AAAGAAAAAA GGAAGTGGAA TGGGTAACTC |
| 1201 | TTCTTGATTA AAAGTTATGT AATAACCAAA TGCAATGTGA AATATTTTAC TGGACTCTTT |
| 1261 | TGAAAAACCA TCTGTAAAAG ACTGGGGTGG GGGTGGGAGG CCAGCACGGT GGTGAGGCAG |
| 1321 | TTGAGAAAAT TTGAATGTGG ATTAGATTTT GAATGATATT GGATAATTAT TGGTAATTTT |
| 1381 | ATGGCCTGTG AGAAGGGTGT TGTAGTTTAT AAAAGACTGT CTTAATTTGC ATACTTAAGC |
| 1441 | ATTTAGGAAT GAAGTGTTAG AGTGTCTTAA AATGTTTCAA ATGGTTTAAC AAAATGTATG |
| 1501 | TGAGGCGTAT GTGGCAAAAT GTTACAGAAT CTAACTGGTG GACATGGCTG TTCATTGTAC |
| 1561 | TGTTTTTTTC TATCTTCTAT ATGTTTAAAA GTATATAATA AAAATATTTA ATTTTTTTTT |
| 1621 | A. |

Conservative nucleotide substitutions of SMN DNA are also contemplated (e.g., a guanine to adenine change at position 625 of GenBank Accession Number NM_000344.2). In some embodiments, the genome lacks AAV rep and cap DNA, that is, there is no AAV rep or cap DNA between the ITRs of the genome. SMN polypeptides contemplated include, but are not limited to, the human SMN1 polypeptide set out in NCBI protein database number NP_000335.1. In embodiments the SMN DNA comprises a polynucleotide which encodes a human SMN polypeptide (for example the human SMN protein identified by Uniprot accession number Q16637, isoform 1 (Q16637-1)). Also contemplated is the SMN1-modifier polypeptide plastin-3 (PLS3) [Oprea et al., Science 320(5875): 524-527 (2008)]. Sequences encoding other polypeptides may be substituted for the SMN DNA.

### Pharmaceutical Compositions

In various embodiments, the virus particles of the present disclosure (referred to as viral particles) can be provided in pharmaceutical compositions suitable for intrathecal administration. The compositions may be provided in formulations comprising one or more inactive ingredient and/or one or more additional active ingredient in addition to the viral particles. In some embodiments, the compositions of the disclosure can be formulated in formulations suitable for intrathecal administration in a mammalian subject, e.g., a human, using components and techniques known in the art.

In some embodiments, the pharmaceutical formulation comprises (a) an AAV9 viral vector comprising a polynucleotide encoding a survival motor neuron (SMN) protein, (b) a Tris buffer, (c) magnesium chloride, (d) sodium chloride, and (e) a poloxamer (e.g., poloxamer 188), wherein the pharmaceutical composition does not comprise a preservative. In one embodiment of the formulation, the AAV9 viral vector further comprises a modified AAV2 ITR, a chicken beta-actin (CB) promoter, a cytomegalovirus (CMV) immediate/early enhancer, a modified SV40 late 16s intron, a Bovine growth hormone (BGH) polyadenylation signal, and an unmodified AAV2 ITR. In one embodiment of the formulation, the Tris buffer concentration is about 10-30 nM, e.g., about 20 mM. In one embodiment, the pH of the formulation is about 7.7 to about 8.3, e.g., about pH 8.0 (e.g., as measured by USP <791> (incorporated by reference in its entirety)). In one embodiment of the formulation, the magnesium chloride concentration is about 0.5-1.5 mM, e.g, about 1 mM. In one embodiment of the formulation, the sodium chloride concentration is about 100-300 mM, e.g., about 200 mM. In one embodiment, the formulation comprises about 0.001-0.15% w/v Poloxamer 188, e.g., about 0.005% w/v poloxamer 188. In some embodiments, the formulation comprises about 1-8×10¹³ vg/mL, e.g., about 1.9-4.2×10¹³ vg/mL of the AAV9 viral vector. In some embodiments, the formulation comprises about 1-8×10¹³ vg/mL and the AAV9 viral vector is administered in a unit dose of about 6.0×10¹³ vg. In some embodiments, the formulation comprises about 1.9-4.2×10¹³ vg/mL and the AAV9 viral vector is administered in a unit dose of about 6.0×10¹³ vg. In some embodiments, the formulation comprises about 1-8×10¹³ vg/mL and the AAV9 viral vector is administered in a unit dose of about 1.2×10¹⁴ vg. In some embodiments, the formulation comprises about 1.9-4.2×10¹³ vg/mL and the AAV9 viral vector is administered in a unit dose of about 1.2×10¹⁴ vg. In some embodiments, the formulation comprises about 1-8×10¹³ vg/mL and the AAV9 viral vector is administered in a unit dose of about 2.4×10¹⁴ vg. In some embodiments, the formulation comprises about 1.9-4.2×10¹³ vg/mL and the AAV9 viral vector is administered in a unit dose of about 2.4×10¹⁴ vg.

When formulated as a solution or suspension, the delivery system may comprise an acceptable carrier, e.g., an aqueous carrier. A variety of aqueous carriers may be used, e.g., water, buffered water, and/or saline. The formulation may also comprise tonicifiers to render the solution iso-osmotic or isotonic, e.g., NaCl, sugars, mannitol and the like. The formulation may also comprise surfactants to stabilize the composition against interfaces and shear, e.g., polysorbate 20, polysorbate 80 and the like. The formulation may be buffered to maintain optimal pH and stability, e.g., using acetate, succinate, citrate, histidine, phosphate or Tris buffers and the like. These compositions may be sterilized using sterilization techniques, or may be sterile filtered. The resulting aqueous solutions may be packaged for use as is, or lyophilized, the lyophilized preparation being combined with a sterile solution prior to administration.

The compositions, e.g., pharmaceutical compositions, may contain pharmaceutically acceptable auxiliary substances to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. In some embodiments, a pharmaceutical composition comprises a preservative. In some other embodiments, a pharmaceutical composition does not comprise a preservative.

In some embodiments, the pharmaceutical composition optionally also comprises one or more additional active or inactive components, e.g., a contrast agent (e.g., Omnipaque™ 180). In some embodiments, the pharmaceutical composition comprises a viral vector comprising an SMN polynucleotide disclosed herein and also comprises a contrast agent (e.g., Omnipaque™, or iohexol-containing agent). In some such embodiments, the contrast agent is premixed with the pharmaceutical composition. In some other embodiments, the contrast agent is not premixed with the pharmaceutical composition. In some embodiments, the contrast agent is mixed with the pharmaceutical composition just prior to intrathecal administration. In some embodiments, the contrast agent (e.g., Omnipaque™, iohexol, and the like) increases motor neuron transduction. In some embodiments, the contrast agent (e.g., Omnipaque™, iohexol, and the like) helps guide the intrathecal needle into the subarachnoid space.

In some embodiments, the contrast medium is administered in combination with a viral vector comprising an SMN polynucleotide disclosed herein, wherein the contrast medium is not premixed with or coformulated with the viral vector prior to administration. For example, in some embodiments, a contrast medium and a viral vector comprising an SMN polynucleotide disclosed herein are administered sequentially. In some embodiments, the contrast medium is mixed with the viral vector comprising an SMN polynucleotide immediately prior to administration as a single bolus.

In some embodiments, a pharmaceutical composition may be prepared and purified according to methods known in the art, e.g., those described in PCT/US2018/058744, which is incorporated herein by reference in its entirety. In some embodiments, a pharmaceutical composition has less than about 7% empty capsids (e.g., 7%, 6%, 5%, 4%, 3%, 2%, 1% or fewer, or any percentage in between of empty capsids), e.g., as assessed by, e.g., qPCR or ddPCR. In some embodiments, a pharmaceutical composition has one or more of the following purity features: less than 0.09 ng of benzonase per 1.0×10¹³ vg, less than 30 μg/g (ppm) of cesium, about 20-80 ppm of Poloxamer 188, less than 0.22 ng of BSA per 1.0×10¹³ vg, less than 6.8×10⁵ pg of residual plasmid DNA per 1.0×10¹³ vg, less than 1.1×10⁵ pg of residual hcDNA per 1.0×10¹³ vg, and less than 4 ng of rHCP per 1.0×10¹³ vg.

In various embodiments, a pharmaceutical composition retains a potency of between +/−20%, between +/−15%, between +/−10%, or between +/−5%, of a reference standard. In one embodiment, the potency is assessed against a reference standard using the methods in Foust et al., Nat. Biotechnol., 28(3), pp. 271-274 (2010). Any suitable reference standard may be used. In one embodiment, the pharmaceutical composition has an in vivo potency, as tested by SMAΔ7 mice. In an embodiment, a tested mouse given a 7.5×10¹³ vg/kg dose has a median survival of greater than 15 days, greater than 20 days, greater than 22 days or greater than 24 days. In one embodiment, the pharmaceutical composition has a potency, as tested by an in vitro cell-based assay, of 50-150%, 60-140% or 70-130% of a reference standard and/or suitable control.

In some embodiments, a pharmaceutical composition has rAAV viral vectors at a concentration between about 1×10¹³ vg/mL and 1×10¹⁵ vg/mL, e.g., between about 1-8×10¹³ vg/mL. In some embodiments, the pharmaceutical composition has less than about 10%, less than about 8%, less than about 7%, or less than about 5% empty viral capsids. In some embodiments, the pharmaceutical composition has less than about 100 ng/mL host cell protein per 1×10¹³ vg/mL. In some embodiments, the pharmaceutical composition has less than about 5×10⁶ pg/mL, less than about 1×10⁶ pg/mL, less than about 7.5×10⁵ pg/mL, or less than 6.8×10⁵ pg/mL residual host cell DNA (hcDNA) per 1×10¹³ vg/mL. In some embodiments, the pharmaceutical composition has less than about 10 ng, less than about 8 ng, less than about 6 ng, or less than about 4 ng of residual host cell protein (rHCP) per 1.0×10¹³ vg/mL. In some embodiments, at least about 50%, at least about 60%, at least about 70%, at least about 80%, at least about 90%, at least about 95%, or at least about 100% of the rAAV (e.g., AAV9) viral vector genomes/mL in the pharmaceutical composition are functional. In some embodiments, the pharmaceutical composition has residual plasmid DNA of less than or equal to 1.7×10⁶ pg/ml per 1×10¹³ vg/ml, or 1×10⁵ pg/ml per 1×10¹³ vg/ml to 1.7×10⁶ pg/ml per 1×10¹³ vg/ml. In some embodiments, the pharmaceutical composition has benzonase concentrations of less than 0.2 ng per 1.0×10¹³ vg, less than 0.1 ng per 1.0×10¹³ vg, or less than 0.09 ng per 1.0×10¹³ vg. In some embodiments, the pharmaceutical composition has bovine serum albumin (BSA) concentrations of less than 0.5 ng per 1.0×10¹³ vg, less than 0.3 ng per 1.0×10¹³ vg, or less than 0.22 ng per 1.0×10¹³ vg. In some embodiments, the pharmaceutical composition has endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or, less than about 0.02 EU/mL per 1.0×10¹³ vg/mL. In some embodiments, the pharmaceutical composition has concentrations of cesium less than 100 μg/g (ppm), less than 50 μg/g (ppm), or less than 30 μg/g (ppm). In some embodiments, the methods yield rAAV viral vectors that have about 10-100 ppm, 15-90 ppm, or about 20-80 ppm of Poloxamer 188. In some embodiments, the pharmaceutical composition has fewer than 2000, fewer than 1500, fewer than 1000 or fewer than 600 particles that are 25 μm in size per container. In some embodiments, the pharmaceutical composition has fewer than 10000, fewer than 8000, fewer than 1000 or fewer than 6000 particles that are 0 μm in size per container. In some embodiments, the pharmaceutical composition has pH of between 7.5 to 8.5, between 7.6 to 8.4 or between 7.8 to 8.3. In some embodiments, the pharmaceutical composition has osmolality of between 330 to 490 mOsm/kg, between 360 to 460 mOsm/kg or between 390 to 430 mOsm/kg. In some embodiments, the pharmaceutical composition has infectious titer of about 1.0×10⁸-10.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg. In some embodiments, the pharmaceutical composition has about 30-150%, about 60-140%, or about 70-130% relative potency based on an in vitro cell-based assay relative to a reference standard and/or suitable control. In some embodiments, the pharmaceutical composition has total protein levels of about 10-500 μg per 1.0×10¹³ vg, about 50-400 μg per 1.0×10¹³ vg, or about 100-300 μg per 1.0×10¹³ vg. In some embodiments, the pharmaceutical composition has an in vivo potency as determined by median survival in an SMNΔ7 mouse given at a 7.5×10¹³ vg/kg dose of greater than 15 days, greater than 20 days, greater than 22 days or greater than 24 days. In some embodiments, the pharmaceutical composition meets a combination of one or more (e.g., all) of the preceding criteria.

The disclosure herein also provides a kit for treating SMA, e.g., Type II or Type III SMA, in a patient in need thereof, wherein the kit comprises one or more doses of a pharmaceutical composition disclosed herein, e.g., one comprising an effective amount or dose of a viral vector comprising an SMN polynucleotide disclosed herein and optionally also comprising one or more additional active or inactive component, e.g., a contrast agent (e.g., Omnipaque™ 180), and instructions on how to use the pharmaceutical preparation or composition. In some embodiments, the kit comprises one or more doses of a pharmaceutical composition disclosed herein, e.g., one comprising an effective amount or dose of a viral vector comprising an SMN polynucleotide disclosed herein and also optionally comprising a contrast agent (e.g., Omnipaque™, or iohexol-containing agent).

In some embodiments, a kit comprises a contrast agent premixed in the same container as the pharmaceutical composition. In some embodiments, a kit comprises contrast agent provided in one or more containers in the kit and the pharmaceutical composition provided in one or more additional containers. In some embodiments, the contrast agent is mixed with the pharmaceutical composition prior to intrathecal administration.

In some embodiments, the kit contains one or more vials of a viral vector pharmaceutical composition. In some embodiments, each vial contains the viral vector pharmaceutical composition at a dose (e.g., a unit dose) of up to or at about 6.0×10¹³ vg. In some embodiments, each vial of a viral vector (e.g., each unit dose) of the kit contains the pharmaceutical composition at a dose of about 6.0×10¹³ vg. In some embodiments, each vial of a viral vector (e.g., each unit dose) of the kit contains the pharmaceutical composition at a dose of up to or at about 1.2×10¹⁴ vg. In some embodiments, each vial of a viral vector (e.g., each unit dose) of the kit contains the pharmaceutical composition at a dose of about 1.2×10¹⁴ vg. In some embodiments, each vial of a viral vector (e.g., each unit dose) of the kit contains the pharmaceutical composition at a dose of up to or at about 2.4×10¹⁴ vg. In some embodiments, each vial of a viral vector (e.g., each unit dose) of the kit contains the pharmaceutical composition at a dose of about 2.4×10¹⁴ vg. In some embodiments, the viral vector pharmaceutical composition is at a concentration of about 0.1-5.0×10¹³ vg/mL. In some embodiments, each vial contains a single dose of rAAV viral vector. In some embodiments, each vial contains more than a single dose of rAAV viral vector. In some embodiments, each vial contains less than a single dose of rAAV viral vector.

Uses of rAAV9 Viral Vector

In various embodiments, disclosed herein are methods for delivery of a polynucleotide to a patient in need of treatment for SMA, e.g., SMA type II or III, comprising administering a rAAV9 with a genome including an rAAV SMN polynucleotide. In some embodiments, the delivery is intrathecal delivery to the central nervous system of a patient, comprising administering a rAAV9 disclosed herein. In some embodiments, the rAAV9 is administered with a contrast agent. In some such embodiments, the rAAV9 and contrast agent are administered simultaneously, for example, in a single pharmaceutical composition. In other such embodiments, the rAAV9 and contrast agent are administered sequentially. For example, in some embodiments, a contrast medium is administered first and the rAAV9 is administered subsequent to administration of the contrast medium. In some embodiments, the rAAV9 is administered first and the contrast medium is administered subsequent to the administration of the AAV9 viral vector. In embodiments where the AAV9 viral vector and contrast medium are administered sequentially, the administration of the AAV9 viral vector and the contrast medium may be administered within, e.g., about 2 hours, within 1 hour, within 45 minutes, within 30 minutes, within 15 minutes, within 10 minutes or within 5 minutes of each other. In some embodiments, at least one of the contrast agent and rAAV9 is administered intrathecally. In some embodiments, both the contrast agent and rAAV9 (whether administered simultaneously or sequentially) are administered intrathecally.

In some embodiments, the contrast agent is a non-ionic, low-osmolar contrast agent. In some embodiments, the contrast agent may increase transduction of target cells in the central nervous system of the patient. In some embodiments, the contrast agent may help to target the delivery directly to the subarachnoid space. In some embodiments, the rAAV9 genome is a self-complementary genome. In other embodiments, the rAAV9 genome is a single-stranded genome.

In some embodiments, the rAAV viral vector is intrathecally delivered into the spinal canal or the subarachnoid space so that it reaches the cerebrospinal fluid (CSF). In some embodiments, the rAAV viral vector may diffuse within the CSF to regions distal to the site of delivery. In some embodiments, the rAAV viral vector is delivered to a brain region. In some embodiments, the rAAV viral vector is delivered to the motor cortex and/or the brain stem. In some embodiments, the rAAV viral vector is delivered to the spinal cord. In some embodiments, the rAAV viral vector is delivered to a lower motor neuron. Embodiments of the disclosure employ rAAV9 to deliver rAAV viral vector to nerve and glial cells. In some embodiments, the glial cell is a microglial cell, an oligodendrocyte or an astrocyte. In some embodiments, the rAAV9 is used to deliver a rAAV viral vector to a Schwann cell.

Titers of rAAV viral vector to be administered may vary depending, for example, on the particular rAAV, the mode of administration, the treatment goal, the age and other characteristics of the individual being treated, and the cell type(s) being targeted. Titer may be determined by known methods. Titers of rAAV may range from about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵, or more DNase resistant particles (DRP) per ml. Dosages may also be expressed in units of vector genomes (vg). The genomic titer can be determined using ddPCR as described in this application, in Lock et al., or any other methods known in the art. Dosages may also vary based on the timing of the administration to a human. These dosages of rAAV may range from about 1×10¹¹ vg/kg, about 1×10¹² vg/kg, about 1×10¹³ vg/kg, about 1×10¹⁴ vg/kg, about 1×10¹⁵ vg/kg, about 1×10¹⁶ vg/kg, or more vector genomes per kilogram body weight in an adult or neonate.

In some embodiments, the rAAV9 is administered at a dose of 1.0×10¹³ vg-9.9×10¹⁴ vg. In some embodiments, the rAAV9 is administered at a dose of 5.0×10¹³ vg-3.0×10¹⁴ vg. In some embodiments, the rAAV9 is administered at a dose of up to 6.0×10¹³ vg. In some embodiments, the rAAV9 is administered at a dose of about 6.0×10¹³ vg. In some embodiments, the rAAV9 is administered at a dose of up to 1.2×10¹⁴ vg. In some embodiments, the rAAV9 is administered at a dose of about 1.2×10¹⁴ vg. In some embodiments, the rAAV9 is administered at a dose of up to 2.4×10¹⁴ vg. In some embodiments, the rAAV9 is administered at a dose of about 2.4×10¹⁴ vg.

In some embodiments, the rAAV9 is administered in a unit dose of about 1.0×10¹³ vg-9.9×10¹⁴ vg. In some embodiments, the rAAV9 is administered in a unit dose of about 1.0×10¹³ vg-5.0×10¹⁴ vg. In some embodiments, the rAAV9 is administered in a unit dose of about 5.0×10¹³ vg-3.0×10¹⁴ vg.

In some embodiments, the rAAV9 is administered in a unit dose of about 6.0×10¹³ vg. some embodiments, the rAAV9 is administered in a unit dose of about 1.2×10¹⁴ vg. some embodiments, the rAAV9 is administered in a unit dose of about 2.4×10¹⁴ vg.

The dose can be determined by any suitable method. For example, PCR with primers specific to the viral vector can provide relative measurements, while qPCR may be used for smaller samples and absolute measurements. In some embodiments, ddPCR is used. ddPCR is a method for performing digital PCR that is based on water-oil emulsion droplet technology. Baker et al., “Digital PCR hits its stride.” Nature Methods, 9(6):541-544. Sykes et al., “Quantitation of targets for PCR by use of limiting dilution.” Biotechniques, 13(3)444-449. A sample is fractionated into tens of thousands of droplets, and PCR amplification of the template molecules occurs in each individual droplet. One does not necessarily need to make a standard curve or have primers with high amplification efficiency, hence ddPCR does not typically use as much sample as traditional PCR-based techniques. Examples of commercially available ddPCR machines include, but are not limited to, the BioRad QX100 ddPCR and the RainDance Raindrop Digital PCR. In one embodiment, the dose is determined using PCR. In another embodiment, the dose is determined using qPCR. In another embodiment, the dose is determined using digital droplet PCR (ddPCR). In some embodiments, multiple methods are used. In some embodiments, the PCR-based methods detect and quantify encapsidated AAV9 viral genome using specifically designed primers and probes targeting the SMN gene. In other embodiments, the PCR-based methods detect and quantify encapsidated AAV9 viral genome using specifically designed primers and probes targeting the chicken beta-actin promoter. In other embodiments, the PCR-based methods detect and quantify encapsidated AAV9 viral genome using specifically designed primers and probes targeting the CMV enhancer. In other embodiments, the PCR-based methods detect and quantify encapsidated AAV9 viral genome using specifically designed primers and probes targeting the ITR sequences. In other embodiments, the PCR-based methods detect and quantify encapsidated AAV9 viral genome using specifically designed primers and probes targeting the bovine growth hormone polyadenylation signal. In some embodiments, potency is measured using a suitable in vitro cellular assay or in vivo animal model. For example, the potency or % functional AAV SMN viral particles may be determined using an animal model of SMA, e.g., the SMNΔ7 mouse, or a quantitative cell-based assay using a suitable cell line, e.g., primary neural progenitor cells (NPCs) isolated from the cortex of SMAΔ7 mice. In one embodiment, the potency is assessed as against a reference standard using the methods in Foust et al., Nat. Biotechnol., 28(3), pp. 271-274 (2010). Any suitable reference standard may be used. In addition, exemplary methods for determining the dose, purity and percentage of functional viral vectors of the rAAV viral vectors disclosed herein are also provided in the disclosure of PCT/US2018/058744, which is incorporated herein by reference in its entirety.

Formulation of rAAV viral vector to be administered may vary depending, for example, on the method of intrathecal administration, the dose volume, and the pharmaceutical excipient. Grouls et al, “General considerations in the formulation of drugs for spinal delivery.” Spinal Drug Delivery, Chapter 15, Elsevier Science, Yaksh edition. In some embodiments, the rAAV viral vector may be administered in a therapeutic formulation suitable for intrathecal administration. In some embodiments, rAAV viral vector may be intrathecally administered as a bolus injection. In some embodiments, the rAAV viral vector may be intrathecally administered as a slow infusion. In some embodiments, the rAAV viral vector may be formulated in a sterile isotonic drug solution. In some embodiments, the rAAV viral vector may be formulated in saline solution. In some embodiments, the rAAV viral vector may be formulated in an artificial CSF, e.g., Elliott's B solution. In some embodiments, therapeutic formulation is filtered before administration.

In various embodiments, the methods and materials disclosed herein are indicated for and can be used in the treatment of SMA, e.g., by intrathecal administration to a patient lacking a functional copy of SMN1. Humans also carry a second nearly identical copy of the SMN gene called SMN2. Lefebvre et al. “Identification and characterization of a spinal muscular atrophy-determining gene.” Cell, 80(1):155-65. Monani et al. “Spinal muscular atrophy: a deficiency in a ubiquitous protein; a motor-neuron specific disease.” Neuron, 48(6):885-896. Both the SMN1 and SMN2 genes express SMN protein, however SMN2 contains a translationally silent mutation in exon 7, which results in inefficient inclusion of exon 7 in SMN2 transcripts. Thus, SMN2 produces both full-length SMN protein and a truncated version of SMN lacking exon 7, with the truncated version as the predominant form. As a result, the amount of functional full-length protein produced by SMN2 is much less (by 70-90%) than that produced by SMN1. Lorson et al. “A single nucleotide in the SMN gene regulates splicing and is responsible for spinal muscular atrophy.” PNAS, 96(11) 6307-6311. Monani et al, “A single nucleotide difference that alters splicing patterns distinguishes the SMA gene SMN1 from the copy gene SMN2.” Hum Mol Genet 8(7):1177-1183. Although SMN2 cannot completely compensate for the loss of the SMN1 gene, patients with milder forms of SMA generally have higher SMN2 copy numbers. Lefebvre et al., “Correlation between severity and SMN protein level in spinal muscular atrophy.” Nat Genet 16(3):265-269. Park et al., “Spinal muscular atrophy: new and emerging insights from model mice.” Curr Neurol Neurosci Rep 10(2):108-117. More than 95% of individuals with SMA retain at least one copy of the SMN2 gene. A caveat is that SMN2 copy number is not the sole phenotypic modifier. In particular, the c.859G>C variant in exon 7 of the SMN2 gene has been reported as a positive disease modifier. Patient with this particular mutation have less severe disease phenotypes. Prior et al., “A positive modified of spinal muscular atrophy in the SMN2 gene.” Am J Hum Genet 85(3):408-413. In some embodiments, the rAAV SMN disclosed herein is administered to Type II SMA patients with more than one copy, more than two copies, more than three copies, more than four copies or more than five copies of the SMN2 gene and/or lacking a c.859G>C variant in exon 7 of the SMN2 gene. In some embodiments, the rAAV SMN disclosed herein is administered to Type III SMA patients with more than two copies, more than three copies, more than four copies or more than five copies of the SMN2 gene and/or lacking a c.859G>C variant in exon 7 of the SMN2 gene. In some embodiments, the rAAV SMN disclosed herein is intrathecally administered to Type II SMA patients. In some embodiments, the rAAV SMN disclosed herein is intrathecally administered to Type III SMA patients.

Type I SMA (also called infantile onset or Werdnig-Hoffmann disease) is when SMA symptoms are present at birth or by the age of 6 months. In this type, babies typically have low muscle tone (hypotonia), a weak cry and breathing distress. They often have difficulty swallowing and sucking, and do not reach the developmental milestone of being able to sit up unassisted. They often show one or more of the SMA symptoms selected from hypotonia, delay in motor skills, poor head control, round shoulder posture and hypermobility of joints. Typically, these babies have two copies of the SMN2 gene, one on each chromosome 5. Over half of all new SMA cases are SMA type I. For Type I SMA, about 80% of patients have 1 or 2 copies of the SMN2 gene.

Type II or intermediate SMA is when SMA has its onset between the ages of 7 and 18 months and before the child can stand or walk independently. Children with Type II SMA generally have at least three SMN2 genes, and about 82% of Type II SMA patients have 3 copies of the SMN2 genes. Late-onset SMA (also known as types III and IV SMA, mild SMA, adult-onset SMA and Kugelberg-Welander disease) results in variable levels of weakness. Type III SMA has its onset after 18 months, and children can stand and walk independently, although they may require aid. Among Type III SMA patients, about 96% have 3 or 4 copies of the SMN2 genes. Type IV SMA has its onset in adulthood, and people are able to walk during their adult years. People with types III or IV SMA generally have between four and eight SMN2 genes, from which a fair amount of full-length SMN protein can be produced.

In one embodiment, rAAV, e.g., rAAV9 vectors disclosed herein, can be administered intrathecally to treat SMA, e.g., SMA type II or type III. The terms “treat,” “treatment,” and other related forms of the term comprise a step of administering, e.g., intrathecally, an effective dose, or effective multiple doses, of a composition comprising a rAAV as disclosed herein to an animal (including a human being) in need thereof. If the dose is administered prior to onset of symptoms of a disorder/disease, the administration is prophylactic. If the dose is administered after the development of a disorder/disease, the administration is therapeutic. In embodiments, an effective dose is a dose that partially or fully alleviates (i.e., eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Examples of disease states contemplated for treatment are set out herein.

In one embodiment, rAAV9 compositions of the disclosure are administered intrathecally to a patient in need of treatment for SMA, e.g., Type II or Type III SMA.

In some embodiments, the patient is 0-72 months of age. In some other embodiments, the patient is 6-60 months of age. In some embodiments, the patient is 6-24 months of age. In some embodiments, the patient is at least 6 months of age. In some embodiments, the patient is greater than 24 months of age.

In some embodiments, the patient has one or more mutations, e.g., a null mutation, in one copy of the SMN1 gene (encompassing any mutation that renders the encoded SMN1 protein nonfunctional). In some embodiments, the patient has one or more mutations, e.g., a null mutation, in two copies of the SMN1 gene. In some embodiments, the patient has one or more mutations, e.g., a null mutation, in all copies of the SMN1 gene. In some embodiments, the patient has a deletion in one copy of the SMN1 gene. In some embodiments, the patient has a deletion in two copies of the SMN1 gene. In some embodiments, the patient has biallelic SMN1 mutations, that is, either a deletion or substitution of SMN1 in both alleles of the chromosome. In some embodiments, the patient has at least one functional copy of the SMN2 gene. In some embodiments, the patient has at least two functional copies of the SMN2 gene. In some embodiments, the patient has at least three functional copies of the SMN2 gene. In some embodiments, the patient has at least four functional copies of the SMN2 gene. In some embodiments, the patient has at least five functional copies of the SMN2 gene. In some embodiments, the patient has bi-allelic SMN1 null mutations or deletions and has three copies of SMN2. In some embodiments, the patient does not have a c.859G>C substitution in exon 7 of at least one copy of the SMN2 gene. In some embodiments, the patient has bi-allelic SMN1 null mutations or deletions, has three copies of SMN2, and does not have a c.859G>C substitution in exon 7 of at least one copy of the SMN2 gene. In some embodiments, the genetic sequence of the SMN1 or SMN2 gene may be determined by, e.g., hybridization, PCR amplification, and/or partial or full chromosome or genome sequencing. In other embodiments, the genetic sequence and copy number of the SMN1 or SMN2 gene may be determined by high-throughput sequencing. In some embodiments, the genetic sequence and copy number of the SMN1 or SMN2 gene may be determined by microarray analysis. In some embodiments, the genetic sequence and copy number of the SMN1 or SMN2 gene may be determined by Sanger sequencing. In some embodiments, the copy number of the SMN1 or SMN2 gene may be determined by fluorescence in-situ hybridization (FISH).

In some embodiments, the patient has been or concurrently is diagnosed with SMA, e.g., SMA Type II or Type III prior to treatment, e.g., by a genomic test and/or a motor function test and/or a physical examination. In some embodiments, SMA Type II or Type III is diagnosed by clinical evaluation of symptoms, e.g. CHOP INTEND, Bayley Scales of Infant and Toddler Development®, or Hammersmith Functional Motor Scale-Expanded (HFMSE). In some embodiments, SMA Type II or Type III is diagnosed by a physical examination. In some embodiments, a Type II SMA patient as treated by the methods disclosed herein is or shows onset of disease symptoms before 24 months, 22 months, 20 months, 18 months, 16 months, 14 months, 12 months, 10 months, 8 months, or 6 months of age, or any age in between. In some embodiments, a Type III SMA patient as treated by the methods disclosed herein is or shows onset of disease symptoms after 12 months, 14 months, 16 months, 18 months, 20 months, 22 months, or 24 months of age, or any age in between. In some embodiments, patients are treated before they show symptoms of Type II or Type III SMA (e.g., one or more symptoms), and instead the patient is determined to need treatment, e.g., using one of the genetic tests described herein. In some embodiments, patients are treated after they show symptoms of Type II or Type III SMA (e.g., one or more symptoms), e.g., as determined using one of the tests described herein. In some embodiments, patients are treated before they show symptoms of Type II or Type III SMA. In some embodiments, patients are diagnosed with Type II or Type III SMA based on genetic testing, before they are symptomatic.

In some embodiments, the patient shows one or more SMA symptoms. SMA symptoms can include hypotonia, delay in motor skills, poor head control, round shoulder posture and hypermobility of joints. In some embodiments, poor head control is determined by placing the patient in a ring sit position with assistance given at the shoulders (front and back). Head control is assessed by the patient's ability to hold the head upright. In some embodiments, spontaneous movement is observed when the patient is in a supine position and motor skills is assessed by the patient's ability to lift their elbows, knees, hands and feet off the surface. In some embodiments, the patient's grip strength is measured by placing a finger in the patient's palm and lifting the patient until their shoulder comes off the surface. Hypotonia and grip strength is measured by how soon/long the patient maintains grasp. In some embodiments, head control is assessed by placing the patient's head in a maximum available rotation and measuring the patient's ability to turn head back towards midline. In some embodiments, shoulder posture may be assessed by sitting patient down with head and trunk support, and observing if patient flexes elbows or shoulder to reach for a stimulus that is placed at shoulder level at arms-length. In some embodiments, shoulder posture may also be assessed by placing patient in a side-lying position, and observing if patient flexes elbows or shoulder to reach for a stimulus that is placed at shoulder level at arms-length. In some embodiments, motor skills are assessed by observing if the patients flex their hips or knees when their foot is stroked, tickled or pinched. In some embodiments, shoulder flexion, elbow flexion, hip adduction, neck flexion, head extension, neck extension, and/or spinal incurvation may be assessed by known clinical measures, e.g., CHOP INTEND. Other SMA symptoms may be evaluated according to known clinical measures, e.g., CHOP INTEND.

In some embodiments, the patient shows the ability to sit but not walk. In some embodiments, the patient has the shows the ability to sit unassisted for 10 or more seconds but cannot stand or walk. In some embodiments, the patient shows the ability to sit unassisted with head erect for 10 or more seconds but cannot walk or stand. In some embodiments, the patient shows the ability of sitting independent as defined by the World Health Organization Multicentre Growth Reference Study (WHO-MGRS) criteria.

Without being bound by theory, intrathecal administration may allow drugs to bypass the blood-brain-barrier. As a result, for drugs where the central nervous system is the target, direct delivery by intrathecal administration may allow for reduced total dose and/or volume of pharmaceutical composition needed (e.g., as compared to IV administration), thereby reducing the risk of hepatotoxicity. Furthermore, direct delivery into the subarachnoid space may allow for higher transduction efficiency of cells in the central nervous system, e.g., lower motor neurons, glia cells and the like. The volume of cerebrospinal fluid (CSF) in the subarachnoid space may influence effective dose concentration chosen for intrathecal delivery. Since CSF volume in a human remains relatively constant after about the age of 3 years, the dose in a patient can be controlled more easily and uniformly across different patients. In some embodiments, intrathecal administration is used to pass through the blood-brain-barrier. In some embodiments, an rAAV9 viral vector disclosed herein is delivered intrathecally to a patient in need thereof, e.g., one identified as in need of treatment for SMA type II or type III. In some embodiments, the rAAV9 is injected into the spinal canal. In some embodiments, the rAAV9 is injected into the subarachnoid space. In some embodiments, the rAAV9 viral vector is injected under sterile conditions in a PICU patient room, or other appropriate settings (e.g., interventional suite, operating room, dedicated procedure room) with immediate access to acute critical care management. In some embodiments, patient vitals are monitored about every 15±5 minutes for 4 hours, and every hour±15 minutes for 24 hours after administration of the viral vector. In some embodiments, the rAAV9 viral vector does not comprise a preservative. In some embodiments, sedation or anesthesia is given to patients prior to administration of the rAAV9 viral vector. In some embodiments, intrathecal administration of rAAV9 viral vector may be performed on patients placed in a prone position, in a knee-chest position, in a lateral position, in a Sim's position, or in a lateral decubitus position. In some embodiments, the rAAV9 viral vector is administered in a syringe, or in a catheter. In some embodiments, a catheter may be inserted into the L1-L2, L2-L3, L3-L4, or L4-L5 interspinous space into the subarachnoid space. In some embodiments, a lumbar puncture is performed, collecting up to 10 mL, up to 9 mL, up to 8 mL, up to 7 mL, up to 6 mL, up to 5 mL, up to 4 mL, up to 3 mL, up to 2 mL or up to 1 mL of cerebrospinal fluid. In some embodiments, the rAAV9 viral vector is injected directly into the subarachnoid space. In some embodiments, the rAAV9 viral vector is premixed with an appropriate radiographic contrast solution (e.g., metrizamide, iopamidol, iohexol, ioversol, Omnipaque™ etc.) and injected directly into the subarachnoid space. In some embodiments, a contrast solution (e.g., metrizamide, iopamidol, iohexol, ioversol, Omnipaque™ etc.) is administered intrathecally prior to intrathecal administration of the rAAV9 viral vector. In some embodiments, the contrast solution is administered intrathetically within 2 hours, within 1 hour, within 45 minutes, within 30 minutes, within 15 minutes, within 10 minutes or within 5 minutes before intrathecal administration of the rAAV9 viral vector. In some embodiments, a contrast solution (e.g., metrizamide, iopamidol, iohexol, ioversol, Omnipaque™ etc.) is administered intrathecally after intrathecal administration of the rAAV9 viral vector. In some embodiments, the contrast solution is administered intrathetically within 2 hours, within 1 hour, within 45 minutes, within 30 minutes, within 15 minutes, within 10 minutes or within 5 minutes after intrathecal administration of the rAAV9 viral vector.

In some embodiments, the volume of contrast agent administered is up to about 0.5 mL, up to about 1.0 mL, up to about 1.5 mL, up to about 2.0 mL, or up to about 2.5 mL. In some embodiments, the total volume administered (rAAV9 viral vector and contrast agent) is no more than about 5 mL, no more than about 6 mL, no more than about 7 mL, no more than about 8 mL, no more than about 9 mL, or no more than about 10 mL. In some embodiments, the patient is placed in a different position following administration of rAAV9 viral vector. In some embodiments, the patient is placed in a Trendelenburg position, or tilted head-down at 20°-40°, e.g., 30°, following administration of the rAAV9 viral vector. In some embodiments, the patient is placed in a Trendelenburg position, or tilted head-down at 30° for 10-30 minutes, e.g., about 15 minutes, following administration of the rAAV9 viral vector.

In some embodiments, treatment is effective in preventing, reducing, alleviating, slowing and/or partially or fully reversing one or more symptom of SMA, e.g., SMA type II or type III. The efficacy of the treatment method may be determined using a variety of tests for motor skills before and after treatment. In particular, the Bayley Scales of Infant and Toddler Development® is a standard series of measurements used to assess the development of infants and toddlers. Bayley N. “Bayley Scales of Infant and Toddler Development.” 3^{rd} edition, Harcourt Assessment Inc., 2006. In particular, the Motor Scale component of Version III (Third Edition) of the Bayley Scales® measures gross and fine motor skills like grasping, sitting, stacking blocks and climbing stairs. In some embodiments, the patient is assessed as to whether their hands are fisted a majority of the time. In some embodiments, the patient is assessed to see if their eyes follow a moving person. In some embodiments, the patient is assessed as to whether he/she purposely attempts to place his/her hand in mouth. In some embodiments, the patient is assessed to see whether he/she holds his/her hands open most of the time when not attempting a task. In some embodiments, the patient is assessed to see if he/she can freely rotate his/her wrist from palm down to palm up when manipulating a small object. In some embodiments, the patient is given blocks and assessed to see if the patient picks up blocks using one or both hands, transfers block from hand to hand, grasps block with pad of thumb or fingertip, and whether the patient grasps the block with thumb partially opposed to fingers. In some embodiments, the patient is given a food pellet and assessed to see if he/she grasps block with pad of thumb or fingertip, and whether the patient grasps the block with thumb partially opposed to fingers. In some embodiments, the patient is given a book and assessed to see if the patient attempts to turn a page or several pages at once. In some embodiments, the patient is given a crayon or pencil and paper and assessed to see if the patient grasps the crayon or pencil using a palmar grasp, a static tripod grasp, or a quadruped grasp while making a mark on the paper. In further embodiments, the patient is assessed to see if his/her grasps is mature, controlled and dynamic while making a mark on the paper. In some embodiments, the patient is assessed to see if he/she holds the paper in place with one hand while scribbling or drawing with the other.

In some embodiments, the patient is assessed to see if he/she thrusts his/her arms or legs several times while in play. In some embodiments, the patient is assessed to see if he/she can intermittently lift his/her head free of a support. In some embodiments, the patient is assessed to see if he/she can hold his/her head erect for at least 3 seconds without support. In some embodiments, the patient is assessed to see if he/she has the ability to walk at least 5 steps with coordination and balance. In some embodiments, the patient is assessed to see if he/she has the ability to walk at least 5 steps with coordination and balance, in accordance with item 43 of the Bayley®-III—Gross Motor. In some embodiments, the patient is assessed to see if he/she has the ability to stand without assistance or support surface, and whether he/she has feedback postural control. In some embodiments, the patient is assessed to see if he/she has the ability to stand without assistance, in accordance with item 40 of the Bayley®-III—Gross Motor. In some embodiments, a patient is considered to have received effective treatment if the patient achieves the ability to stand without support at about 24 months, 12 months, 9 months, or 6 months after administration of treatment. In some embodiments, a patient is considered to have received effective treatment if the patient achieves the ability to walk without assistance, as defined by taking at least five steps independently displaying coordination and balance at about 24 months, 12 months, 9 months, or 6 months after administration of treatment.

Another commonly used measure of infant development is the Hammersmith Functional Motor Scale-Expanded (HFMSE). O'Hagen et al., “An expanded version of the Hammersmith Functional Motor Scale for SMA II and III patients.” Neuromuscul Disord, 17(9-10):693-7; Glanzman et al., “Validation of the Expanded Hammersmith Functional Motor Scale in spinal muscular atrophy type II and III.” J Child Neurol, 26(12):1499-1507. While the Hammersmith Functional Motor Scale was successful in assessing the ability of non-ambulant individuals with SMA, the HFMSE provided an additional 13-item add-on that could successfully distinguish motor skills among individuals with SMA Type II and Type III. In some embodiments, the patient is assessed for his/her ability to sit on a chair or a floor unsupported. In some embodiments, the patient is assessed for his/her ability to touch a hand to his/her head while sitting unsupported on a chair or a floor. In some embodiments, the patient is assessed for his/her ability to touch both hands to his/her head while sitting unsupported on a chair or a floor. In some embodiments, the patient is assessed as to whether he/she can roll to the side while lying down. In some embodiments, the patient is assessed as to whether he/she can roll face-up to face down or vice versa while lying down. In some embodiments, the patient is assessed as to whether he/she can lie down from a sitting position in a controlled manner. In some embodiments, the patient is assessed as to whether he/she can prop up on forearms while prone. In some embodiments, the patient is assessed as to whether he/she can lift his/her head up while in a prone position. In some embodiments, the patient is assessed as to whether he/she can prop up with straight arms for a count of 3 while prone. In some embodiments, the patient is assessed as to whether he/she can get from a lying to a sitting position without rolling onto his/her tummy. In some embodiments, the patient is assessed as to whether he/she can get onto his/her hands and knees while keeping the head up for a count of 3. In some embodiments, the patient is assessed as to whether he/she can crawl forwards on the hands and knees. In some embodiments, the patient is assessed as to whether he/she can lift his/her head while lying supine with arms folded across the chest. In some embodiments, the patient is assessed as to whether he/she can stand for a count of 3 with one hand or no hands as a support. In some embodiments, the patient is assessed as to whether he/she can walk without any help. In some embodiments, the patient is assessed as to whether he/she can bring either knee to chest while lying supine. In some embodiments, the patient is assessed as to whether he/she can get from a high kneel position to a half kneel position without using arms. In some embodiments, the patient is assessed as to whether he/she can get to a standing position from a high kneel position without using arms. In some embodiments, the patient is assessed as to whether he/she can get from a standing position to a sitting position without using arms. In some embodiments, the patient is assessed as to whether he/she can get from a standing position to a squatting position without using arms. In some embodiments, the patient is assessed as to whether he/she can jump forward 12 inches from a standing position. In some embodiments, the patient is assessed as to whether he/she can walk up or down 4 steps with no help or with the help of one railing. In some embodiments, a patient is considered to have received effective treatment if the patient exhibits a 5-10 point increase, e.g., an 8-point increase, from baseline on the HFMSE at about 24 months, 12 months, 9 months, or 6 months after administration of treatment. In some embodiments, a patient is considered to have received effective treatment if the patient exhibits a 9-point increase from baseline on the HFMSE at about 24 months, 12 months, 9 months, or 6 months after administration of treatment. In some embodiments, a patient is considered to have received effective treatment if the patient exhibits a 10-point increase from baseline on the HFMSE at about 24 months, 12 months, 9 months, or 6 months after administration of treatment.

In some embodiments, the efficacy of treatment is measured by changes in development abilities. In some embodiments, a baseline measurement is taken before administration of the rAAV9 viral vector. In some embodiments, the baseline measurement comprises measuring the fine and gross motor components of the Bayley Scales of Infant and Toddler Development®. In some embodiments, the baseline measurement comprises measuring item 43 (take at least 5 steps with no assistance) of the gross motor components of the Bayley Scales of Infant and Toddler Development®. In some embodiments, the baseline measurement comprises measuring item 40 (stand without support for at least 3 seconds) of the gross motor components of the Bayley Scales of Infant and Toddler Development®. In some embodiments, the baseline measurement comprises assessing the patient according to the Hammersmith Functional Motor Scale-Expanded (HFMSE). In some embodiments, the efficacy of treatment is assessed by measuring item 43 (take at least 5 steps with no assistance) of the gross motor components of the Bayley Scales of Infant and Toddler Development® and comparing to baseline. In some embodiments, the efficacy of treatment is assessed by measuring item 40 (stand without support for at least 3 seconds) of the gross motor components of the Bayley Scales of Infant and Toddler Development® and comparing to baseline. In some embodiments, the efficacy of treatment is assessed by assessing the patient on the HFMSE and comparing to baseline before treatment. In some embodiments, the baseline is established by measurements within 30 days before treatment. In some embodiments, the efficacy of treatment is assessed within 30 days of treatment. In some embodiments, the efficacy of treatment is assessed once a month for twelve months after treatment. In some embodiments, the assessments of efficacy is videotaped. In some embodiments, significant motor milestones are assessed by a standard Motor Milestone Development Survey shown in Table 2. In some embodiments, the efficacy of treatment is assessed at least 12 months after, at least 24 months after, at least 48 months after, at least 72 months after, or up 10 years after treatment.

TABLE 2
Motor Milestone Development Survey

| Developmental Milestone - Bayley | |
|---|---|
| Scale ® Item Number | Performance Criteria |
| Head Control - Gross Motor Subtest | Child holds head erect for at least 3 |
| Item #4 | seconds without support |
| Rolls from Back to Sides - Gross Motor | Child turns from back to both right and |
| Subtest Item #20 | left sides |
| Sits Without Support - Gross Motor | Child sits alone without support for at |
| Subtest Item #26 | least 30 seconds |
| Stands with Assistance - Gross Motor | Child supports own weight for at least 2 |
| Subtest Item #33 | seconds |
| Crawls - Gross Motor Subtest Item #34 | Child makes forward progress of at |
| | least 5 feet by crawling on hands and |
| | knees |
| Pulls to Stand - Gross Motor Subtest | Child raises self to standing position |
| Item #35 | using chair or other convenient object |
| | for support |
| Walks with Assistance - Gross Motor | Child walks by making coordinated, |
| Subtest Item #37 | alternated stepping movements |
| Stands Alone - Gross Motor Subtest | Child stands alone for at least 3 |
| Item #40 | seconds after you release his or her |
| | hands |
| Walks Alone - Gross Motor Subtest Item | Child takes at least five steps |
| #43 | independently, displaying coordination |
| | and balance |

In some embodiments, testing to evaluate treatment efficacy is not limited to the Bayley Scales of Infant and Toddler Development®, the Hammersmith Functional Motor Scale-Expanded (HFMSE), or the Motor Milestone Development Survey, but may also include other motor skills tests known in the art, including but not limited to CHOP INTEND, TIMP, CHOP TOSS, the Peabody Development Motor Scales, the Brazelton Neonatal Behavior Assessment test, Ability Captured Through Interactive Video Evaluation (ACTIVE), and measurements of compound motor action potentials (CMAP).

The pre-screening of patients amenable to treatment is also contemplated, e.g., according to the methods of identifying SMA, e.g., SMA type II or type III disclosed herein, as well as the administration of treatment to patients identified according to criteria disclosed herein.

AAVs may give rise to both a cellular and humoral immune response. As a result, a fraction of potential patients for AAV-based gene therapy harbors pre-existing antibodies against AAV. Jeune et al., “Pre-existing anti-Adeno-Associated Virus antibodies as a challenge in AAV gene therapy.” Hum Gene Ther Methods, 24(2):59-67. Boutin et al., “Prevalence of serum IgG and neutralizing factors against adeno-associated virus (AAV) types 1, 2, 5, 6, 8, and 9 in the healthy population: implications for gene therapy using AAV vectors.” Hum Gene Ther, 21:704-712. Because even very low levels of antibodies can prevent successful transduction, antecedent anti-AAV antibodies pose a serious obstacle to the universal application of AAV gene therapy. In some embodiments, the levels of anti-AAV9 antibody titers in a patient is determined prior to administration of the AAV viral vector and the patient is given the AAV by intrathecal administration only if antibody titers are below a threshold level. In some embodiments, the levels of anti-AAV9 antibody titers in a patient is determined by an ELISA binding immunoassay. In some embodiments, the patient has anti-AAV9 antibody titers at or below 1:100 as determined by an ELISA binding immunoassay prior to administration of treatment. In some embodiments, the patient has anti-AAV9 antibody titers at or below 1:50 as determined by an ELISA binding immunoassay prior to administration of treatment. In some embodiments, the patient has anti-AAV9 antibody titers above 1:100 as determined by an ELISA binding immunoassay after treatment and is monitored for 1-8 weeks or until titers decrease to below 1:100. In some embodiments, the patient has anti-AAV9 antibody titers above 1:100 as determined by an ELISA binding immunoassay after treatment and is monitored for 1-8 weeks or until titers decrease to below 1:50.

In some embodiments, patients with high anti-AAV antibody titer may be administered one or more immunosuppressant drugs. For example, monoclonal anti-CD20 antibodies such as rituximab, in combination with cyclosporine A, may bring down anti-AAV titers. Mingozzi et al., “Pharmacological modulation of humoral immunity in a nonhuman primate model of AAV gene transfer for hemophilia B.” Mol Ther, 20:1410-1416. In some embodiments, the patient has anti-AAV9 antibody titers above 1:100 as determined by an ELISA binding immunoassay prior to or after treatment and is treated with one or more immunosuppressant drugs, e.g. steroids like prednisolone. In some embodiments, the patient has anti-AAV9 antibody titers above 1:50 as determined by an ELISA binding immunoassay prior to or after treatment and is treated with one or more immunosuppressant drugs, e.g. steroids like prednisolone.

In some embodiments, a patient with high anti-AAV antibody titer may be subjected to plasmapheresis to deplete neutralizing antibodies prior to and/or after vector administration. Monteilhet et al., “A 10 patient case report on the impact of plasmapheresis upon neutralizing factors against adeno-associated virus (AAV) types 1, 2, 6, and 8.” Mol Ther, 19(11):2084-2091. During plasmapheresis, blood is withdrawn from a patient and the plasma and blood cells are separated by either centrifugation or hollow fiber filtration. The blood cells are then returned to the patient together with either treated plasma or replacement fluids, such as a 4.5% human albumin in saline. A common use of therapeutic apheresis is the removal of undesired immunoglobulins such as anti-AAV antibodies. In some embodiments, the patient has anti-AAV9 antibody titers above 1:100 as determined by an ELISA binding immunoassay prior to or after treatment and is treated with plasmapheresis. In some embodiments, the patient has anti-AAV9 antibody titers above 1:50 as determined by an ELISA binding immunoassay prior to or after treatment and is treated with plasmapheresis.

Pre-existing maternal antibodies to AAV9 may be transferred to a young patient through breast milk or placental transfer in utero. In some embodiments, the patient has anti-AAV9 antibody titers above 1:100 as determined by an ELISA binding immunoassay prior to or after treatment and is switched to formula feeding. In some embodiments, the patient has anti-AAV9 antibody titers above 1:50 as determined by an ELISA binding immunoassay prior to or after treatment and is switched to formula feeding.

Prior to and after administration of treatment, the condition of the patient may be monitored. In some embodiments, a patient who have received an AAV-based treatment may experience low platelet counts or thrombocytopenia, which is a condition characterized by particularly low platelet count. Thrombocytopenia can be detected by a full blood count using a diluted sample of blood on a hemocytometer. Thrombocytopenia can also be detected by viewing a slide prepared with the patient's blood (a thin blood film or peripheral smear) under the microscope. Normal human platelet counts range from 150,000 cells/ml to about 450,000 cells/ml.

In some embodiments, the patient has platelet counts above about 67,000 cells/ml prior to administration or above about 100,000 cells/ml, or above about 150,000 cells/ml. In some embodiments, the patient has platelet counts below about 150,000 cells/ml prior to administration or below about 100,000 cells/ml, or below about 67,000 cells/ml, and is monitored for 1-8 weeks or until platelet counts increase to above about 67,000 cells/ml, or above about 100,000 cells/ml, or above about 150,000 cells/ml. In some embodiments where platelet counts are below about 67,000 cells/ml after administration of the viral vector, the patient may be treated with platelet transfusion. In some embodiments, the patient does not have thrombocytopenia prior to administration of the viral vector. In some embodiments, the patient has thrombocytopenia after administration of the viral vector and is monitored for about 1-8 weeks or until the patient does not have thrombocytopenia. In some embodiments, the patient has thrombocytopenia after administration of the viral vector and is treated with a platelet transfusion.

Monitoring the condition of patients may also involve standard blood tests that measure levels of one or more of platelets, serum protein electrophoresis, serum gamma-glutamyl transferase (GGT), aspartate transaminase (AST) and alanine aminotransferase (ALT), total bilirubin, glucose, creatine kinase (CK), creatinine, blood urea nitrogen (BUN), electrolytes, alkaline phosphatase and amylase. Troponin I levels are a general measure for heart health, and elevated levels reflect heart damage or heart-related conditions. In some embodiments, troponin-I levels are monitored after administration of the viral vector. In some embodiments, patients may have troponin-I levels less than about 0.3, 0.2, 0.15, or 0.1 μg/ml before administration of the viral vector. In some embodiments, patients may have troponin-I levels less than about 0.176 μg/ml before administration of the viral vector. In some embodiments, patients may have troponin-I levels above about 0.176 μg/ml after administration of the viral vector. In some embodiments, patients receive cardiac monitoring after administration of the viral vector until troponin-I levels are less than about 0.176 μg/ml.

Aspartate transaminase (AST) and alanine aminotransferase (ALT) and total bilirubin are a general measure of hepatic function, while creatinine tracks renal function. Elevated levels of AST, ALT or total bilirubin may indicate hepatic malfunction. In some embodiments, the patient has normal hepatic function prior to administration of the viral vector. In some embodiments, the patient has hepatic transaminase levels less than about 8-40 U/L prior to administration of the viral vector. In some embodiments, the patient has AST or ALT levels less than about 8-40 U/L prior to administration of the viral vector. In some embodiments, the patient has gamma-glutamyl transferase (GGT) less than 3 times upper limit of normal, e.g., as determined by clinical standards and methods known in the art, e.g., CLIA standards. In some embodiments, the patient has bilirubin levels less than 3.0 mg/dL prior to administration of the viral vector. In some embodiments, patients have creatinine levels less than 1.8 mg/dL, less than 1.4 mg/dL, or less than 1.0 mg/dL prior to administration of the viral vector. In some embodiments, patients have hemoglobin (Hgb) levels between 8-18 g/dL prior to administration of the viral vector. In some embodiments, the patient has white blood cell (WBC) counts less than 20000 per mm³ prior to administration of the viral vector.

In various embodiments, gene therapy using AAV vectors as described herein may produce an antigen specific T-cell response to the AAV vector, e.g., between 2-4 weeks following gene transfer. One possible consequence to such antigen specific T-cell response is clearance of the transduced cells and loss of transgene expression. In an attempt to dampen the host immune response to the AAV based therapy, patients may be given immune suppressants. In some embodiments, T-cell response may be measured by ELISPOT assay. In some embodiments, T-cell response prior to administering the vector is 100 spot forming cells (SFC) per 10⁶ peripheral blood mononuclear cells (PBMCs). In some embodiments, patients may be given glucocorticoids before administration of viral vector. In some embodiments, patients may be given a corticosteroid before administration of viral vector. In some embodiments, patients may be given an oral steroid before administration of viral vector. Examples of oral steroids include but are not limited to prednisone, prednisolone, methylprednisolone, triamcinolone, bethamethasone, dexamethasone and hydrocortisone. In some embodiments, the oral steroid is or comprises prednisolone.

In some embodiments, the patient is started on prophylactic steroid at least 12-48 hours, e.g., at least 24 hours, prior to administering the viral vector. In some embodiments, the patient is given oral steroid for at least 10-60 days, e.g., at least 30 days, after administering the viral vector. In some embodiments, the oral steroid is administered once daily. In some embodiments, the oral steroid is administered twice daily. In some embodiments, the oral steroid is given at a dose of about 0.1-10 mg/kg, e.g, about 1 mg/kg. In some embodiments, the oral steroid is given at a dose of about 0.1-10 mg/kg/day, e.g., about 1 mg/kg/day. In some embodiments, the levels of AST and ALT are monitored after administration of the viral vector. In such embodiments, the oral steroid treatment is administered when AST and ALT levels exceed twice the upper limit of normal, e.g., as determined by clinical standards and methods known in the art, or about 120 IU/L. In some embodiments, the oral steroid treatment is administered for more than 30 days and for as long as AST and ALT levels exceed twice the upper limit of normal, e.g., as determined by clinical standards and methods known in the art, or for as long as levels exceed about 120 IU/L. In some embodiments, the oral steroid treatment is administered for more than 30 days as long as T-cell response is above 100 SFC per 10⁶ PBMCs. In some embodiments, the oral steroid treatment is administered for more than 30 days until T-cell response falls below 100 SFC per 10⁶ PBMCs. During sustained treatment with corticosteroids, the adrenal glands naturally decrease production of cortisol. If corticosteroid treatment is stopped abruptly, the body may experience cortisol deficiency. In some embodiments where oral steroid is given to a patient for at least 30 days, the steroid dose is slowly tapered on a schedule. In some embodiments, the oral steroid dose is tapered when AST and ALT levels fall below twice the upper limit of normal, e.g., as determined by clinical standards and methods known in the art, or about 120 IU/L. In some embodiments, tapering comprises stepped decrements to 0.5 mg/kg/day for about 2 weeks followed by 0.25 mg/kg/day for about 2 more weeks. In some other embodiments, tapering of the oral steroid occurs at the discretion of the doctor. In some embodiments, blood samples are collected and test for serum antibodies to AAV9 by ELISA, serum antibodies to SMN by ELISA, or interferon gamma (IFN-g) by ELISpots.

Methods of selecting patients who will benefit from the treatment disclosed here are also contemplated herein. In some embodiments, the patient is not contraindicated for spinal tap procedure, or administration of intrathecal therapy. In some embodiments, the patient does not have scoliosis, or severe scoliosis, e.g., as defined by a 50° curvature of spine that is evident on an X-ray examination. In some embodiments, the patient does not have a previous, planned, or expected scoliosis repair surgery or procedure scheduled within 2 years, within 1 year or within 6 months of administration of the rAAV9 viral vector. In some embodiments, the patient does not need invasive ventilatory support, or a gastric feeding tube. In some embodiments, the patient does not have a history of standing or walking independently. In some embodiments, the patient does not have an active viral infection at the time of administration of the rAAV9 viral vector. In further embodiments, these viral infections include but are not limited to human immunodeficiency virus (HIV) or serology positive hepatitis B or C or the Zika virus. In some embodiments, the patient does not have concomitant illness, for example major renal or hepatic impairment, known seizure disorder, diabetes mellitus, idiopathic hypocalciuria or symptomatic cardiomyopathy. In some embodiments, the patient does not have severe non-pulmonary infections or respiratory tract infections (e.g., pyelonephritis or meningitis) within four weeks of administration of rAAV9 viral vector. In some embodiments, the patient does not have a history of bacterial meningitis, brain or spinal cord disease. In some embodiments, the patient does not have a known allergy or hypersensitivity to gluococorticosteroids, e.g. prednisone or prednisolone, or their excipients. In some embodiments, the patient does not have a known allergy or hypersensitivity to iodine or iodine-containing products. In some embodiments, the patient is not concomitantly taking drugs for treating myopathy or neuropathy. In some embodiments, the patient is not receiving immunosuppressive therapy, plasmapheresis, immunomodulators such as adalimumab within three months of administration of rAAV9 viral vector.

Combination therapies are also contemplated herein. Combination as used herein includes either simultaneous treatment or sequential treatments. Combinations of methods can include the addition of certain standard medical treatments (e.g., riluzole in ALS), and/or combinations with novel therapies. For example, other therapies for SMA that may be used in the disclosed combination therapies include antisense oligonucleotides (ASOs) that alter bind to pre-mRNA and alter their splicing patterns. Singh. et al., “A multi-exon-skipping detection assay reveals surprising diversity of splice isoforms of spinal muscular atrophy genes.” Plos One, 7(11):e49595. In some embodiments, nusinersen (U.S. Pat. Nos. 8,361,977 and 8,980,853, incorporated herein by reference) may be used. Nusinersen is an approved ASO that target intron 6, exon 7 or intron 7 of SMN2 pre-mRNA, modulating the splicing of SMN2 to more efficiently produce full-length SMN protein. In some embodiments, the method of treatment comprising the AAV9 viral vector is administered in combination with a muscle enhancer. In some embodiments, a disclosed method of treatment comprises administering an AAV9 viral vector in combination with a neuroprotector. In some embodiments, a disclosed method of treatment comprises administering an AAV9 viral vector in combination with an antisense oligonucleotide-based drug targeting SMN. In some embodiments, a disclosed method of treatment comprises administering an AAV9 viral vector in combination with nusinersen. In some embodiments, a disclosed method of treatment comprises administering an AAV9 viral vector in combination with a myostatin-inhibiting drug. In some embodiments, a disclosed method of treatment comprises administering an AAV9 viral vector in combination with stamulumab. In some embodiments, a disclosed method of treatment comprises administering an AAV9 viral vector in combination with more than one additional treatment.

The rAAV viral vectors disclosed herein can be prepared according to preparation and purification methods known in the art. In some embodiments, the purification methods seek to remove contaminants from host cells and chemicals added during the harvesting of viral vectors. In some embodiments, the methods disclosed in PCT/US2018/058744 are used, and that PCT is incorporated herein by reference in its entirety. In some embodiments, the methods yield rAAV viral vectors at a concentration between about 1×10¹³ vg/mL and 1×10¹⁵ vg/mL, e.g., between about 1-8×10¹³ vg/mL. In some embodiments, the methods yield rAAV viral vectors at a dose (e.g., a unit dose) of about 1.0×10¹³ vg-9.9×10¹⁴ vg. In some embodiments, the methods yield rAAV viral vectors at a dose (e.g., a unit dose) of about 1.0×10¹³ vg-5.0×10¹⁴ vg. In some embodiments, the methods yield rAAV viral vectors at a dose (e.g., a unit dose) of about 5.0×10¹³ vg-3.0×10¹⁴ vg. In some embodiments, the methods yield rAAV viral vectors at a dose (e.g., a unit dose) of about 6.0×10¹³ vg. In some embodiments, the methods yield rAAV viral vectors at a dose (e.g., a unit dose) of about 1.2×10¹⁴ vg. In some embodiments, the methods yield rAAV viral vectors at a dose (e.g., a unit dose) of about 2.4×10¹⁴ vg.

In some embodiments, the methods yield rAAV viral vectors that have less than about 10%, less than about 8%, less than about 7%, or less than about 5% empty viral capsids. In some embodiments, the methods yield rAAV viral vectors that have less than about 100 ng/mL host cell protein per 1×10¹³ vg/mL. In some embodiments, the methods yield rAAV viral vectors that have less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL. In some embodiments, the methods yield rAAV viral vectors that have less than about 10 ng, less than about 8 ng, less than about 6 ng, or less than about 4 ng of residual host cell protein (rHCP) per 1.0×10¹³ vg/mL. In some embodiments, the methods yield at least about 50%, at least about 60%, at least about 70%, at least about 80%, at least about 90%, at least about 95%, or at least about 100% of the rAAV (e.g., AAV9) viral vector genomes/mL that are functional. In some embodiments, the methods yield rAAV viral vectors that have residual plasmid DNA of less than or equal to 1.7×10⁶ μg/ml per 1×10¹³ vg/ml, or 1×10⁵ μg/ml per 1×10¹³ vg/ml to 1.7×10⁶ μg/ml per 1×10¹³ vg/ml. In some embodiments, the methods yield rAAV viral vectors that have benzonase concentrations of less than 0.2 ng per 1.0×10¹³ vg, less than 0.1 ng per 1.0×10¹³ vg, or less than 0.09 ng per 1.0×10¹³ vg. In some embodiments, the methods yield rAAV viral vectors that have bovine serum albumin (BSA) concentrations of less than 0.5 ng per 1.0×10¹³ vg, less than 0.3 ng per 1.0×10¹³ vg, or less than 0.22 ng per 1.0×10¹³ vg. In some embodiments, the methods yield rAAV viral vectors that have endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL. In some embodiments, the methods yield rAAV viral vectors that have concentrations of cesium less than 100 μg/g (ppm), less than 50 μg/g (ppm), or less than 30 μg/g (ppm). In some embodiments, the methods yield rAAV viral vectors that have about 10-100 ppm, 15-90 ppm, or about 20-80 ppm of Poloxamer 188. In some embodiments, the methods yield rAAV viral vectors that have fewer than 2000, fewer than 1500, fewer than 1000 or fewer than 600 particles that are 25 μm in size per container. In some embodiments, the methods yield rAAV viral vectors that have fewer than 10000, fewer than 8000, fewer than 1000 or fewer than 6000 particles that are 0 μm in size per container. In some embodiments, the methods yield rAAV viral vectors that have pH of between 7.5 to 8.5, between 7.6 to 8.4 or between 7.8 to 8.3. In some embodiments, the methods yield rAAV viral vectors that have osmolality of between 330 to 490 mOsm/kg, between 360 to 460 mOsm/kg or between 390 to 430 mOsm/kg. In some embodiments, the methods yield rAAV viral vectors that have infectious titer of about 1.0×10⁸-10.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg. In some embodiments, the methods yield rAAV viral vectors that have about 30-150%, about 60-140%, or about 70-130% relative potency based on an in vitro cell-based assay relative to a reference standard and/or suitable control. In some embodiments, the methods yield rAAV viral vectors that have total protein levels of about 10-500 μg per 1.0×10¹³ vg, about 50-400 μg per 1.0×10¹³ vg, or about 100-300 μg per 1.0×10¹³ vg. In some embodiments, the methods yield rAAV viral vectors that have an in vivo potency as determined by median survival in an SMNΔ7 mouse given at a 7.5×10¹³ vg/kg dose of greater than 15 days, greater than 20 days, greater than 22 days or greater than 24 days.

In any of the above embodiments, the preparation and/or purification method may yield rAAV viral vectors that may be formulated for administration and/or are present in a pharmaceutical composition at a unit dose of about 6.0×10¹³ vg. In any of the above embodiments, the preparation and/or purification method may yield rAAV viral vectors that may be formulated for administration and/or are present in a pharmaceutical composition at a unit dose of about 1.2×10¹⁴ vg. In any of the above embodiments, the preparation and/or purification method may yield rAAV viral vectors that may be formulated for administration and/or are present in a pharmaceutical composition at a unit dose of about 2.4×10¹⁴ vg.

For example, in some embodiments, the methods yield rAAV viral vectors that have less than about 10%, less than about 8%, less than about 7%, or less than about 5% empty viral capsids, wherein the rAAV viral vectors are formulated for administration and/or are present in a pharmaceutical composition at a unit dose of about 6.0×10¹³ vg. In some embodiments, the methods yield rAAV viral vectors that have less than about 10%, less than about 8%, less than about 7%, or less than about 5% empty viral capsids, wherein the rAAV viral vectors are formulated for administration and/or are present in a pharmaceutical composition at a unit dose of about 1.2×10¹⁴ vg. In some embodiments, the methods yield rAAV viral vectors that have less than about 10%, less than about 8%, less than about 7%, or less than about 5% empty viral capsids, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have less than about 10%, less than about 8%, less than about 7%, or less than about 5% empty viral capsids. In some embodiments, a formulation or pharmaceutical composition comprises a unit dosage of rAAV viral vectors of about 1.2×10¹⁴ vg, wherein the rAAV viral vectors have less than about 10%, less than about 8%, less than about 7%, or less than about 5% empty viral capsids. In some embodiments, a formulation or pharmaceutical composition comprises a unit dosage of rAAV viral vectors of about 2.4×10¹⁴ vg, wherein the rAAV viral vectors have less than about 10%, less than about 8%, less than about 7%, or less than about 5% empty viral capsids.

In some embodiments, the methods yield rAAV viral vectors that have less than about 100 ng/mL host cell protein per 1×10¹³ vg/mL and the rAAV viral vectors are formulated for administration and/or are present in a pharmaceutical composition at a unit dose of about 6.0×10¹³ vg. In some embodiments, the methods yield rAAV viral vectors that have less than about 100 ng/mL host cell protein per 1×10¹³ vg/mL and the rAAV viral vectors are formulated for administration and/or are present in a pharmaceutical composition at a unit dose of about 1.2×10¹⁴ vg. In some embodiments, the methods yield rAAV viral vectors that have less than about 100 ng/mL host cell protein per 1×10¹³ vg/mL and the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have less than about 100 ng/mL host cell protein per 1×10¹³ vg/mL. In some embodiments, a formulation or pharmaceutical composition comprises a unit dosage of rAAV viral vectors of about 1.2×10¹⁴ vg, wherein the rAAV viral vectors have less than about 100 ng/mL host cell protein per 1×10¹³ vg/mL. In some embodiments, a formulation or pharmaceutical composition comprises a unit dosage of rAAV viral vectors of about 2.4×10¹⁴ vg, wherein the rAAV viral vectors have less than about 100 ng/mL host cell protein per 1×10¹³ vg/mL.

In some embodiments, the methods yield rAAV viral vectors that have less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL, wherein the rAAV viral vectors are formulated for administration and/or are present in a pharmaceutical composition at a unit dose of about 6.0×10¹³ vg. In some embodiments, the methods yield rAAV viral vectors that have less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL, wherein the rAAV viral vectors are formulated for administration and/or are present in a pharmaceutical composition at a unit dose of about 1.2×10¹⁴ vg. In some embodiments, the methods yield rAAV viral vectors that have less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL. In some embodiments, a formulation or pharmaceutical composition comprises a unit dosage of rAAV viral vectors of about 1.2×10¹⁴ vg, wherein the rAAV viral vectors have less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL. In some embodiments, a formulation or pharmaceutical composition comprises a unit dosage of rAAV viral vectors of about 2.4×10¹⁴ vg, wherein the rAAV viral vectors have less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL.

In some embodiments, the methods yield rAAV viral vectors that have less than about 10 ng, less than about 8 ng, less than about 6 ng, or less than about 4 ng of residual host cell protein (rHCP) per 1.0×10¹³ vg/mL, wherein the rAAV viral vectors are formulated for administration and/or are present in a pharmaceutical composition at a unit dose of about 6.0×10¹³ vg. In some embodiments, the methods yield rAAV viral vectors that have less than about 10 ng, less than about 8 ng, less than about 6 ng, or less than about 4 ng of residual host cell protein (rHCP) per 1.0×10¹³ vg/mL, wherein the rAAV viral vectors are formulated for administration and/or are present in a pharmaceutical composition at a unit dose of about 1.2×10¹⁴ vg. In some embodiments, the methods yield rAAV viral vectors that have less than about 10 ng, less than about 8 ng, less than about 6 ng, or less than about 4 ng of residual host cell protein (rHCP) per 1.0×10¹³ vg/mL, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have less than about 10 ng, less than about 8 ng, less than about 6 ng, or less than about 4 ng of residual host cell protein (rHCP) per 1.0×10¹³ vg/mL. In some embodiments, a formulation or pharmaceutical composition comprises a unit dosage of rAAV viral vectors of about 1.2×10¹⁴ vg, wherein the rAAV viral vectors have less than about 10 ng, less than about 8 ng, less than about 6 ng, or less than about 4 ng of residual host cell protein (rHCP) per 1.0×10¹³ vg/mL. In some embodiments, a formulation or pharmaceutical composition comprises a unit dosage of rAAV viral vectors of about 2.4×10¹⁴ vg, wherein the rAAV viral vectors have less than about 10 ng, less than about 8 ng, less than about 6 ng, or less than about 4 ng of residual host cell protein (rHCP) per 1.0×10¹³ vg/mL.

In some embodiments, the methods yield at least about 50%, at least about 60%, at least about 70%, at least about 80%, at least about 90%, at least about 95%, or at least about 100% of the AAV9 viral vector genomes/mL are functional, wherein the rAAV viral vectors are formulated for administration and/or are present in a pharmaceutical composition at a unit dose of about 6.0×10¹³ vg. In some embodiments, the methods yield at least about 50%, at least about 60%, at least about 70%, at least about 80%, at least about 90%, at least about 95%, or at least about 100% of the AAV9 viral vector genomes/mL are functional, wherein the rAAV viral vectors are formulated for administration and/or are present in a pharmaceutical composition at a unit dose of about 1.2×10¹⁴ vg. In some embodiments, the methods yield at least about 50%, at least about 60%, at least about 70%, at least about 80%, at least about 90%, at least about 95%, or at least about 100% of the AAV9 viral vector genomes/mL are functional, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein about 50%, at least about 60%, at least about 70%, at least about 80%, at least about 90%, at least about 95%, or at least about 100% of the rAAV (e.g, rAAV9) viral vector genomes/mL are functional. In some embodiments, a formulation or pharmaceutical composition comprises a unit dosage of rAAV viral vectors of about 1.2×10¹⁴ vg, wherein about 50%, at least about 60%, at least about 70%, at least about 80%, at least about 90%, at least about 95%, or at least about 100% of the rAAV (e.g, rAAV9) viral vector genomes/mL are functional. In some embodiments, a formulation or pharmaceutical composition comprises a unit dosage of rAAV viral vectors of about 2.4×10¹⁴ vg, wherein the rAAV viral vectors, wherein about 50%, at least about 60%, at least about 70%, at least about 80%, at least about 90%, at least about 95%, or at least about 100% of the rAAV (e.g, rAAV9) viral vector genomes/mL are functional.

In some embodiments, the methods yield rAAV viral vectors that have residual plasmid DNA of less than or equal to 1.7×10⁶ pg/ml per 1×10¹³ vg/ml, or 1×10⁵ pg/ml per 1×10¹³ vg/ml to 1.7×10⁶ pg/ml per 1×10¹³ vg/ml, wherein the rAAV viral vectors are formulated for administration and/or are present in a pharmaceutical composition at a unit dose of about 6.0×10¹³ vg. In some embodiments, the methods yield rAAV viral vectors that have residual plasmid DNA of less than or equal to 1.7×10⁶ pg/ml per 1×10¹³ vg/ml, or 1×10⁵ pg/ml per 1×10¹³ vg/ml to 1.7×10⁶ pg/ml per 1×10¹³ vg/ml, wherein the rAAV viral vectors are formulated for administration and/or are present in a pharmaceutical composition at a unit dose of about 1.2×10¹⁴ vg. In some embodiments, the methods yield rAAV viral vectors that have residual plasmid DNA of less than or equal to 1.7×10⁶ pg/ml per 1×10¹³ vg/ml, or 1×10⁵ pg/ml per 1×10¹³ vg/ml to 1.7×10⁶ pg/ml per 1×10¹³ vg/ml, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have residual plasmid DNA of less than or equal to 1.7×10⁶ pg/ml per 1×10¹³ vg/ml, or 1×10⁵ pg/ml per 1×10¹³ vg/ml to 1.7×10⁶ pg/ml per 1×10¹³ vg/ml. In some embodiments, a formulation or pharmaceutical composition comprises a unit dosage of rAAV viral vectors of about 1.2×10¹⁴ vg, wherein the rAAV viral vectors have residual plasmid DNA of less than or equal to 1.7×10⁶ pg/ml per 1×10¹³ vg/ml, or 1×10⁵ pg/ml per 1×10¹³ vg/ml to 1.7×10⁶ pg/ml per 1×10¹³ vg/ml. In some embodiments, a formulation or pharmaceutical composition comprises a unit dosage of rAAV viral vectors of about 2.4×10¹⁴ vg, wherein the rAAV viral vectors have residual plasmid DNA of less than or equal to 1.7×10⁶ pg/ml per 1×10¹³ vg/ml, or 1×10⁵ pg/ml per 1×10¹³ vg/ml to 1.7×10⁶ pg/ml per 1×10¹³ vg/ml.

In some embodiments, the methods yield rAAV viral vectors that have benzonase concentrations of less than 0.2 ng per 1.0×10¹³ vg, less than 0.1 ng per 1.0×10¹³ vg, or less than 0.09 ng per 1.0×10¹³ vg, wherein the rAAV viral vectors are formulated for administration and/or are present in a pharmaceutical composition at a unit dose of about 6.0×10¹³ vg. In some embodiments, the methods yield rAAV viral vectors that have benzonase concentrations of less than 0.2 ng per 1.0×10¹³ vg, less than 0.1 ng per 1.0×10¹³ vg, or less than 0.09 ng per 1.0×10¹³ vg, wherein the rAAV viral vectors are formulated for administration and/or are present in a pharmaceutical composition at a unit dose of about 1.2×10¹⁴ vg. In some embodiments, the methods yield rAAV viral vectors that have benzonase concentrations of less than 0.2 ng per 1.0×10¹³ vg, less than 0.1 ng per 1.0×10¹³ vg, or less than 0.09 ng per 1.0×10¹³ vg, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have benzonase concentrations of less than 0.2 ng per 1.0×10¹³ vg, less than 0.1 ng per 1.0×10¹³ vg, or less than 0.09 ng per 1.0×10¹³ vg. In some embodiments, a formulation or pharmaceutical composition comprises a unit dosage of rAAV viral vectors of about 1.2×10¹⁴ vg, wherein the rAAV viral vectors have benzonase concentrations of less than 0.2 ng per 1.0×10¹³ vg, less than 0.1 ng per 1.0×10¹³ vg, or less than 0.09 ng per 1.0×10¹³ vg. In some embodiments, a formulation or pharmaceutical composition comprises a unit dosage of rAAV viral vectors of about 2.4×10¹⁴ vg, wherein the rAAV viral vectors have benzonase concentrations of less than 0.2 ng per 1.0×10¹³ vg, less than 0.1 ng per 1.0×10¹³ vg, or less than 0.09 ng per 1.0×10¹³ vg.

In some embodiments, the methods yield rAAV viral vectors that have bovine serum albumin (BSA) concentrations of less than 0.5 ng per 1.0×10¹³ vg, less than 0.3 ng per 1.0×10¹³ vg, or less than 0.22 ng per 1.0×10¹³ vg, wherein the rAAV viral vectors are formulated for administration and/or are present in a pharmaceutical composition at a unit dose of about 6.0×10¹³ vg. In some embodiments, the methods yield rAAV viral vectors that have bovine serum albumin (BSA) concentrations of less than 0.5 ng per 1.0×10¹³ vg, less than 0.3 ng per 1.0×10¹³ vg, or less than 0.22 ng per 1.0×10¹³ vg, wherein the rAAV viral vectors are formulated for administration and/or are present in a pharmaceutical composition at a unit dose of about 1.2×10¹⁴ vg. In some embodiments, the methods yield rAAV viral vectors that have bovine serum albumin (BSA) concentrations of less than 0.5 ng per 1.0×10¹³ vg, less than 0.3 ng per 1.0×10¹³ vg, or less than 0.22 ng per 1.0×10¹³ vg, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have bovine serum albumin (BSA) concentrations of less than 0.5 ng per 1.0×10¹³ vg, less than 0.3 ng per 1.0×10¹³ vg, or less than 0.22 ng per 1.0×10¹³ vg. In some embodiments, a formulation or pharmaceutical composition comprises a unit dosage of rAAV viral vectors of about 1.2×10¹⁴ vg, wherein the rAAV viral vectors have bovine serum albumin (BSA) concentrations of less than 0.5 ng per 1.0×10¹³ vg, less than 0.3 ng per 1.0×10¹³ vg, or less than 0.22 ng per 1.0×10¹³ vg. In some embodiments, a formulation or pharmaceutical composition comprises a unit dosage of rAAV viral vectors of about 2.4×10¹⁴ vg, wherein the rAAV viral vectors have bovine serum albumin (BSA) concentrations of less than 0.5 ng per 1.0×10¹³ vg, less than 0.3 ng per 1.0×10¹³ vg, or less than 0.22 ng per 1.0×10¹³ vg.

In some embodiments, the methods yield rAAV viral vectors that have endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL, wherein the rAAV viral vectors are formulated for administration and/or are present in a pharmaceutical composition at a unit dose of about 6.0×10¹³ vg. In some embodiments, the methods yield rAAV viral vectors that have endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL, wherein the rAAV viral vectors are formulated for administration and/or are present in a pharmaceutical composition at a unit dose of about 1.2×10¹⁴ vg. In some embodiments, the methods yield rAAV viral vectors that have endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL. In some embodiments, a formulation or pharmaceutical composition comprises a unit dosage of rAAV viral vectors of about 1.2×10¹⁴ vg, wherein the rAAV viral vectors have endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL. In some embodiments, a formulation or pharmaceutical composition comprises a unit dosage of rAAV viral vectors of about 2.4×10¹⁴ vg, wherein the rAAV viral vectors have endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL.

In some embodiments, the methods yield rAAV viral vectors that have concentrations of cesium less than 100 μg/g (ppm), less than 50 μg/g (ppm), or less than 30 μg/g (ppm), wherein the rAAV viral vectors are formulated for administration and/or are present in a pharmaceutical composition at a unit dose of about 6.0×10¹³ vg. In some embodiments, the methods yield rAAV viral vectors that have concentrations of cesium less than 100 μg/g (ppm), less than 50 μg/g (ppm), or less than 30 μg/g (ppm), wherein the rAAV viral vectors are formulated for administration and/or are present in a pharmaceutical composition at a unit dose of about 1.2×10¹⁴ vg. In some embodiments, the methods yield rAAV viral vectors that have concentrations of cesium less than 100 μg/g (ppm), less than 50 μg/g (ppm), or less than 30 μg/g (ppm), wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have concentrations of cesium less than 100 μg/g (ppm), less than 50 μg/g (ppm), or less than 30 μg/g (ppm). In some embodiments, a formulation or pharmaceutical composition comprises a unit dosage of rAAV viral vectors of about 1.2×10¹⁴ vg, wherein the rAAV viral vectors have concentrations of cesium less than 100 μg/g (ppm), less than 50 μg/g (ppm), or less than 30 μg/g (ppm). In some embodiments, a formulation or pharmaceutical composition comprises a unit dosage of rAAV viral vectors of about 2.4×10¹⁴ vg, wherein the rAAV viral vectors have concentrations of cesium less than 100 μg/g (ppm), less than 50 μg/g (ppm), or less than 30 μg/g (ppm).

In some embodiments, the methods yield rAAV viral vectors that have about 10-100 ppm, 15-90 ppm, or about 20-80 ppm of Poloxamer 188, wherein the rAAV viral vectors are formulated for administration and/or are present in a pharmaceutical composition at a unit dose of about 6.0×10¹³ vg. In some embodiments, the methods yield rAAV viral vectors that have about 10-100 ppm, 15-90 ppm, or about 20-80 ppm of Poloxamer 188, wherein the rAAV viral vectors are formulated for administration and/or are present in a pharmaceutical composition at a unit dose of about 1.2×10¹⁴ vg. In some embodiments, the methods yield rAAV viral vectors that have about 10-100 ppm, 15-90 ppm, or about 20-80 ppm of Poloxamer 188, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have about 10-100 ppm, 15-90 ppm, or about 20-80 ppm of Poloxamer 188. In some embodiments, a formulation or pharmaceutical composition comprises a unit dosage of rAAV viral vectors of about 1.2×10¹⁴ vg, wherein the rAAV viral vectors have about 10-100 ppm, 15-90 ppm, or about 20-80 ppm of Poloxamer 188. In some embodiments, a formulation or pharmaceutical composition comprises a unit dosage of rAAV viral vectors of about 2.4×10¹⁴ vg, wherein the rAAV viral vectors have about 10-100 ppm, 15-90 ppm, or about 20-80 ppm of Poloxamer 188.

In some embodiments, the methods yield rAAV viral vectors that have fewer than 2000, fewer than 1500, fewer than 1000 or fewer than 600 particles that are ≥25 μm in size per container, wherein the rAAV viral vectors are formulated for administration and/or are present in a pharmaceutical composition at a unit dose of about 6.0×10¹³ vg. In some embodiments, the methods yield rAAV viral vectors that have fewer than 2000, fewer than 1500, fewer than 1000 or fewer than 600 particles that are ≥25 μm in size per container, wherein the rAAV viral vectors are formulated for administration and/or are present in a pharmaceutical composition at a unit dose of about 1.2×10¹⁴ vg. In some embodiments, the methods yield rAAV viral vectors that have fewer than 2000, fewer than 1500, fewer than 1000 or fewer than 600 particles that are ≥25 μm in size per container, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have fewer than 2000, fewer than 1500, fewer than 1000 or fewer than 600 particles that are ≥25 μm in size per container. In some embodiments, a formulation or pharmaceutical composition comprises a unit dosage of rAAV viral vectors of about 1.2×10¹⁴ vg, wherein the rAAV viral vectors have fewer than 2000, fewer than 1500, fewer than 1000 or fewer than 600 particles that are ≥25 μm in size per container. In some embodiments, a formulation or pharmaceutical composition comprises a unit dosage of rAAV viral vectors of about 2.4×10¹⁴ vg, wherein the rAAV viral vectors have fewer than 2000, fewer than 1500, fewer than 1000 or fewer than 600 particles that are ≥25 μm in size per container.

In some embodiments, the methods yield rAAV viral vectors that have fewer than 10000, fewer than 8000, fewer than 1000 or fewer than 6000 particles that are ≥10 μm in size per container, wherein the rAAV viral vectors are formulated for administration and/or are present in a pharmaceutical composition at a unit dose of about 6.0×10¹³ vg. In some embodiments, the methods yield rAAV viral vectors that have fewer than 10000, fewer than 8000, fewer than 1000 or fewer than 6000 particles that are ≥10 μm in size per container, wherein the rAAV viral vectors are formulated for administration and/or are present in a pharmaceutical composition at a unit dose of about 1.2×10¹⁴ vg. In some embodiments, the methods yield rAAV viral vectors that have fewer than 10000, fewer than 8000, fewer than 1000 or fewer than 6000 particles that are ≥10 μm in size per container, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have fewer than 10000, fewer than 8000, fewer than 1000 or fewer than 6000 particles that are ≥10 μm in size per container. In some embodiments, a formulation or pharmaceutical composition comprises a unit dosage of rAAV viral vectors of about 1.2×10¹⁴ vg, wherein the rAAV viral vectors have fewer than 10000, fewer than 8000, fewer than 1000 or fewer than 6000 particles that are ≥10 μm in size per container. In some embodiments, a formulation or pharmaceutical composition comprises a unit dosage of rAAV viral vectors of about 2.4×10¹⁴ vg, wherein the rAAV viral vectors have fewer than 10000, fewer than 8000, fewer than 1000 or fewer than 6000 particles that are ≥10 μm in size per container.

In some embodiments, the methods yield rAAV viral vectors that have pH of between 7.5 to 8.5, between 7.6 to 8.4 or between 7.8 to 8.3, wherein the rAAV viral vectors are formulated for administration and/or are present in a pharmaceutical composition at a unit dose of about 6.0×10¹³ vg. In some embodiments, the methods yield rAAV viral vectors that have pH of between 7.5 to 8.5, between 7.6 to 8.4 or between 7.8 to 8.3, wherein the rAAV viral vectors are formulated for administration and/or are present in a pharmaceutical composition at a unit dose of about 1.2×10¹⁴ vg. In some embodiments, the methods yield rAAV viral vectors that have pH of between 7.5 to 8.5, between 7.6 to 8.4 or between 7.8 to 8.3, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have pH of between 7.5 to 8.5, between 7.6 to 8.4 or between 7.8 to 8.3. In some embodiments, a formulation or pharmaceutical composition comprises a unit dosage of rAAV viral vectors of about 1.2×10¹⁴ vg, wherein the rAAV viral vectors have pH of between 7.5 to 8.5, between 7.6 to 8.4 or between 7.8 to 8.3. In some embodiments, a formulation or pharmaceutical composition comprises a unit dosage of rAAV viral vectors of about 2.4×10¹⁴ vg, wherein the rAAV viral vectors have pH of between 7.5 to 8.5, between 7.6 to 8.4 or between 7.8 to 8.3.

In some embodiments, the methods yield rAAV viral vectors that have osmolality of between 330 to 490 mOsm/kg, between 360 to 460 mOsm/kg or between 390 to 430 mOsm/kg, wherein the rAAV viral vectors are formulated for administration and/or are present in a pharmaceutical composition at a unit dose of about 6.0×10¹³ vg. In some embodiments, the methods yield rAAV viral vectors that have osmolality of between 330 to 490 mOsm/kg, between 360 to 460 mOsm/kg or between 390 to 430 mOsm/kg, wherein the rAAV viral vectors are formulated for administration and/or are present in a pharmaceutical composition at a unit dose of about 1.2×10¹⁴ vg. In some embodiments, the methods yield rAAV viral vectors that have osmolality of between 330 to 490 mOsm/kg, between 360 to 460 mOsm/kg or between 390 to 430 mOsm/kg, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have osmolality of between 330 to 490 mOsm/kg, between 360 to 460 mOsm/kg or between 390 to 430 mOsm/kg. In some embodiments, a formulation or pharmaceutical composition comprises a unit dosage of rAAV viral vectors of about 1.2×10¹⁴ vg, wherein the rAAV viral vectors have osmolality of between 330 to 490 mOsm/kg, between 360 to 460 mOsm/kg or between 390 to 430 mOsm/kg. In some embodiments, a formulation or pharmaceutical composition comprises a unit dosage of rAAV viral vectors of about 2.4×10¹⁴ vg, wherein the rAAV viral vectors have osmolality of between 330 to 490 mOsm/kg, between 360 to 460 mOsm/kg or between 390 to 430 mOsm/kg.

In some embodiments, the methods yield rAAV viral vectors that have infectious titer of about 1.0×10⁸-10.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg, wherein the rAAV viral vectors are formulated for administration and/or are present in a pharmaceutical composition at a unit dose of about 6.0×10¹³ vg. In some embodiments, the methods yield rAAV viral vectors that have infectious titer of about 1.0×10⁸-10.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg, wherein the rAAV viral vectors are formulated for administration and/or are present in a pharmaceutical composition at a unit dose of about 1.2×10¹⁴ vg. In some embodiments, the methods yield rAAV viral vectors that have infectious titer of about 1.0×10⁸-1.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have infectious titer of about 1.0×10⁸-1.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg. In some embodiments, a formulation or pharmaceutical composition comprises a unit dosage of rAAV viral vectors of about 1.2×10¹⁴ vg, wherein the rAAV viral vectors have infectious titer of about 1.0×10⁸-10.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg. In some embodiments, a formulation or pharmaceutical composition comprises a unit dosage of rAAV viral vectors of about 2.4×10¹⁴ vg, wherein the rAAV viral vectors have infectious titer of about 1.0×10⁸-10.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg.

In some embodiments, the methods yield rAAV viral vectors that have about 30-150%, about 60-140%, or about 70-130% relative potency based on an in vitro cell-based assay relative to a reference standard and/or suitable control, wherein the rAAV viral vectors are formulated for administration and/or are present in a pharmaceutical composition at a unit dose of about 6.0×10¹³ vg. In some embodiments, the methods yield rAAV viral vectors that have about 30-150%, about 60-140%, or about 70-130% relative potency based on an in vitro cell-based assay relative to a reference standard and/or suitable control, wherein the rAAV viral vectors are formulated for administration and/or are present in a pharmaceutical composition at a unit dose of about 1.2×10¹⁴ vg. In some embodiments, the methods yield rAAV viral vectors that have about 30-150%, about 60-140%, or about 70-130% relative potency based on an in vitro cell-based assay relative to a reference standard and/or suitable control, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have about 30-150%, about 60-140%, or about 70-130% relative potency based on an in vitro cell-based assay relative to a reference standard and/or suitable control. In some embodiments, a formulation or pharmaceutical composition comprises a unit dosage of rAAV viral vectors of about 1.2×10¹⁴ vg, wherein the rAAV viral vectors have about 30-150%, about 60-140%, or about 70-130% relative potency based on an in vitro cell-based assay relative to a reference standard and/or suitable control. In some embodiments, a formulation or pharmaceutical composition comprises a unit dosage of rAAV viral vectors of about 2.4×10¹⁴ vg, wherein the rAAV viral vectors have about 30-150%, about 60-140%, or about 70-130% relative potency based on an in vitro cell-based assay relative to a reference standard and/or suitable control.

In some embodiments, the methods yield rAAV viral vectors that have total protein levels of about 10-500 μg per 1.0×10¹³ vg, about 50-400 μg per 1.0×10¹³ vg, or about 100-300 μg per 1.0×10¹³ vg, wherein the rAAV viral vectors are formulated for administration and/or are present in a pharmaceutical composition at a unit dose of about 6.0×10¹³ vg. In some embodiments, the methods yield rAAV viral vectors that have total protein levels of about 10-500 μg per 1.0×10¹³ vg, about 50-400 μg per 1.0×10¹³ vg, or about 100-300 μg per 1.0×10¹³ vg, wherein the rAAV viral vectors are formulated for administration and/or are present in a pharmaceutical composition at a unit dose of about 1.2×10¹⁴ vg. In some embodiments, the methods yield rAAV viral vectors that have total protein levels of about 10-500 μg per 1.0×10¹³ vg, about 50-400 μg per 1.0×10¹³ vg, or about 100-300 μg per 1.0×10¹³ vg, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have total protein levels of about 10-500 μg per 1.0×10¹³ vg, about 50-400 μg per 1.0×10¹³ vg, or about 100-300 μg per 1.0×10¹³ vg. In some embodiments, a formulation or pharmaceutical composition comprises a unit dosage of rAAV viral vectors of about 1.2×10¹⁴ vg, wherein the rAAV viral vectors have total protein levels of about 10-500 μg per 1.0×10¹³ vg, about 50-400 μg per 1.0×10¹³ vg, or about 100-300 μg per 1.0×10¹³ vg. In some embodiments, a formulation or pharmaceutical composition comprises a unit dosage of rAAV viral vectors of about 2.4×10¹⁴ vg, wherein the rAAV viral vectors have total protein levels of about 10-500 μg per 1.0×10¹³ vg, about 50-400 μg per 1.0×10¹³ vg, or about 100-300 μg per 1.0×10¹³ vg.

In some embodiments, the methods yield rAAV viral vectors that have an in vivo potency as determined by median survival in an SMNΔ7 mouse given at a 7.5×10¹³ vg/kg dose of greater than 15 days, greater than 20 days, greater than 22 days or greater than 24 days, wherein the rAAV viral vectors are formulated for administration and/or are present in a pharmaceutical composition at a unit dose of about 6.0×10¹³ vg. In some embodiments, the methods yield rAAV viral vectors that have an in vivo potency as determined by median survival in an SMNΔ7 mouse given at a 7.5×10¹³ vg/kg dose of greater than 15 days, greater than 20 days, greater than 22 days or greater than 24 days, wherein the rAAV viral vectors are formulated for administration and/or are present in a pharmaceutical composition at a unit dose of about 1.2×10¹⁴ vg. In some embodiments, the methods yield rAAV viral vectors that have an in vivo potency as determined by median survival in an SMNΔ7 mouse given at a 7.5×10¹³ vg/kg dose of greater than 15 days, greater than 20 days, greater than 22 days or greater than 24 days, wherein the rAAV viral vectors are formulated for administration and/or are present in a pharmaceutical composition at a unit dose of about 2.4×10¹⁴ vg. In some embodiments, a formulation or pharmaceutical composition comprises a unit dosage of rAAV viral vectors of about 6.0×10¹³ vg, wherein the rAAV viral vectors have an in vivo potency as determined by median survival in an SMNΔ7 mouse given at a 7.5×10¹³ vg/kg dose of greater than 15 days, greater than 20 days, greater than 22 days or greater than 24 days. In some embodiments, a formulation or pharmaceutical composition comprises a unit dosage of rAAV viral vectors of about 1.2×10¹⁴ vg, wherein the rAAV viral vectors have an in vivo potency as determined by median survival in an SMNΔ7 mouse given at a 7.5×10¹³ vg/kg dose of greater than 15 days, greater than 20 days, greater than 22 days or greater than 24 days. In some embodiments, a formulation or pharmaceutical composition comprises a unit dosage of rAAV viral vectors of about 2.4×10¹⁴ vg, wherein the rAAV viral vectors have an in vivo potency as determined by median survival in an SMNΔ7 mouse given at a 7.5×10¹³ vg/kg dose of greater than 15 days, greater than 20 days, greater than 22 days or greater than 24 days.

In some embodiments, a formulation or pharmaceutical composition comprises a unit dosage of rAAV viral vectors of about 6.0×10¹³ vg and one or more of the following release criteria: less than about 10%, less than about 8%, less than about 7%, or less than about 5% empty viral capsids; less than about 100 ng/mL host cell protein per 1×10¹³ vg/mL; less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL; less than about 10 ng, less than about 8 ng, less than about 6 ng, or less than about 4 ng of residual host cell protein (rHCP) per 1.0×10¹³ vg/mL; at least about 50%, at least about 60%, at least about 70%, at least about 80%, at least about 90%, at least about 95%, or at least about 100% of rAAV viral vector genomes/mL that are functional; residual plasmid DNA of less than or equal to 1.7×10⁶ μg/mL per 1×10¹³ vg/mL, or 1×10⁵ μg/ml per 1×10¹³ vg/mL to 1.7×10⁶ μg/mL per 1×10¹³ vg/mL; benzonase concentrations of less than 0.2 ng per 1.0×10¹³ vg, less than 0.1 ng per 1.0×10¹³ vg, or less than 0.09 ng per 1.0×10¹³ vg; bovine serum albumin (BSA) concentrations of less than 0.5 ng per 1.0×10¹³ vg, less than 0.3 ng per 1.0×10¹³ vg, or less than 0.22 ng per 1.0×10¹³ vg; endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL; concentrations of cesium less than 100 μg/g (ppm), less than 50 μg/g (ppm), or less than 30 μg/g (ppm); about 10-100 ppm, 15-90 ppm, or about 20-80 ppm of Poloxamer 188; fewer than 2000, fewer than 1500, fewer than 1000 or fewer than 600 particles that are 25 μm in size per container; fewer than 10000, fewer than 8000, fewer than 1000 or fewer than 6000 particles that are 0 μm in size per container; pH of between 7.5 to 8.5, between 7.6 to 8.4 or between 7.8 to 8.3; osmolality of between 330 to 490 mOsm/kg, between 360 to 460 mOsm/kg or between 390 to 430 mOsm/kg; infectious titer of about 1.0×10⁸-10.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg; about 30-150%, about 60-140%, or about 70-130% relative potency based on an in vitro cell-based assay relative to a reference standard and/or suitable control; total protein levels of about 10-500 μg per 1.0×10¹³ vg, about 50-400 μg per 1.0×10¹³ vg, or about 100-300 μg per 1.0×10¹³ vg; an in vivo potency as determined by median survival in an SMNΔ7 mouse given at a 7.5×10¹³ vg/kg dose of greater than 15 days, greater than 20 days, greater than 22 days or greater than 24 days.

In some embodiments, a formulation or pharmaceutical composition comprises a unit dosage of rAAV viral vectors of about 6.0×10¹³ vg and one or more of the following release criteria: pH of about 7.7-8.3; osmolality of about 390-430 mOsm/kg; less than about 600 particles that are 25 μm in size per container; less than about 6000 particles that are 10 μm in size per container, about 1.7×10¹³-5.3×10¹³ vg/mL genomic titer; infectious titer of about 3.9×10⁸-8.4×10¹⁰ IU per 1.0×10¹³ vg; total protein levels of about 100-300 μg per 1.0×10¹³ vg; Pluronic F-68 content of about 20-80 ppm; relative potency of about 70-130% based on an in vitro cell-based assay, wherein the potency is relative to a reference standard and/or suitable control; in vivo potency characterized by median survival in a SMNΔ7 mouse model greater than or equal to 24 days at a dose of 7.5×10¹³ vg/kg; less than about 5% empty capsid; a total purity of greater than or equal to about 95%; less than or equal to about 0.13 EU/mL endotoxin.

In some embodiments, a formulation or pharmaceutical composition comprises a unit dosage of rAAV viral vectors of about 6.0×10¹³ vg and one or more of the following release criteria: less than about 0.09 ng of benzonase per 1.0×10¹³ vg; less than about 30 μg/g (ppm) of cesium; about 20-80 ppm of Poloxamer 188; less than about 0.22 ng of BSA per 1.0×10¹³ vg; less than about 6.8×10⁵ μg of residual plasmid DNA per 1.0×10¹³ vg; less than about 1.1×10⁵ μg of residual hcDNA per 1.0×10¹³ vg; less than about 4 ng of rHCP per 1.0×10¹³ vg; pH of about 7.7-8.3; osmolality of about 390-430 mOsm/kg; less than about 600 particles that are 25 μm in size per container; less than about 6000 particles that are 10 μm in size per container; about 1.7×10¹³-5.3×10¹³ vg/mL genomic titer; infectious titer of about 3.9×10⁸-8.4×10¹⁰ IU per 1.0×10¹³ vg; total protein levels of about 100-300 μg per 1.0×10¹³ vg; relative potency of about 70-130% based on an in vitro cell-based assay, wherein the potency is relative to a reference standard and/or suitable control; less than about 5% empty capsid.

In some embodiments, a formulation or pharmaceutical composition comprises a unit dosage of rAAV viral vectors of about 1.2×10¹⁴ vg and one or more of the following release criteria: less than about 10%, less than about 8%, less than about 7%, or less than about 5% empty viral capsids; less than about 100 ng/mL host cell protein per 1×10¹³ vg/mL; less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL; less than about 10 ng, less than about 8 ng, less than about 6 ng, or less than about 4 ng of residual host cell protein (rHCP) per 1.0×10¹³ vg/mL; at least about 50%, at least about 60%, at least about 70%, at least about 80%, at least about 90%, at least about 95%, or at least about 100% of rAAV viral vector genomes/mL that are functional; residual plasmid DNA of less than or equal to 1.7×10⁶ μg/mL per 1×10¹³ vg/mL, or 1×10⁵ μg/ml per 1×10¹³ vg/mL to 1.7×10⁶ μg/mL per 1×10¹³ vg/mL; benzonase concentrations of less than 0.2 ng per 1.0×10¹³ vg, less than 0.1 ng per 1.0×10¹³ vg, or less than 0.09 ng per 1.0×10¹³ vg; bovine serum albumin (BSA) concentrations of less than 0.5 ng per 1.0×10¹³ vg, less than 0.3 ng per 1.0×10¹³ vg, or less than 0.22 ng per 1.0×10¹³ vg; endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL; concentrations of cesium less than 100 μg/g (ppm), less than 50 μg/g (ppm), or less than 30 μg/g (ppm); about 10-100 ppm, 15-90 ppm, or about 20-80 ppm of Poloxamer 188; fewer than 2000, fewer than 1500, fewer than 1000 or fewer than 600 particles that are 25 μm in size per container; fewer than 10000, fewer than 8000, fewer than 1000 or fewer than 6000 particles that are 0 μm in size per container; pH of between 7.5 to 8.5, between 7.6 to 8.4 or between 7.8 to 8.3; osmolality of between 330 to 490 mOsm/kg, between 360 to 460 mOsm/kg or between 390 to 430 mOsm/kg; infectious titer of about 1.0×10⁸-10.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg; about 30-150%, about 60-140%, or about 70-130% relative potency based on an in vitro cell-based assay relative to a reference standard and/or suitable control; total protein levels of about 10-500 μg per 1.0×10¹³ vg, about 50-400 μg per 1.0×10¹³ vg, or about 100-300 μg per 1.0×10¹³ vg; an in vivo potency as determined by median survival in an SMNΔ7 mouse given at a 7.5×10¹³ vg/kg dose of greater than 15 days, greater than 20 days, greater than 22 days or greater than 24 days.

In some embodiments, a formulation or pharmaceutical composition comprises a unit dosage of rAAV viral vectors of about 1.2×10¹⁴ vg and one or more of the following release criteria: pH of about 7.7-8.3; osmolality of about 390-430 mOsm/kg; less than about 600 particles that are 25 μm in size per container; less than about 6000 particles that are 10 μm in size per container, about 1.7×10¹³-5.3×10¹³ vg/mL genomic titer; infectious titer of about 3.9×10⁸-8.4×10¹⁰ IU per 1.0×10¹³ vg; total protein levels of about 100-300 μg per 1.0×10¹³ vg; Pluronic F-68 content of about 20-80 ppm; relative potency of about 70-130% based on an in vitro cell-based assay, wherein the potency is relative to a reference standard and/or suitable control; in vivo potency characterized by median survival in a SMNΔ7 mouse model greater than or equal to 24 days at a dose of 7.5×10¹³ vg/kg; less than about 5% empty capsid; a total purity of greater than or equal to about 95%; less than or equal to about 0.13 EU/mL endotoxin.

In some embodiments, a formulation or pharmaceutical composition comprises a unit dosage of rAAV viral vectors of about 1.2×10¹⁴ vg and one or more of the following release criteria: less than about 0.09 ng of benzonase per 1.0×10¹³ vg; less than about 30 μg/g (ppm) of cesium; about 20-80 ppm of Poloxamer 188; less than about 0.22 ng of BSA per 1.0×10¹³ vg; less than about 6.8×10⁵ μg of residual plasmid DNA per 1.0×10¹³ vg; less than about 1.1×10⁵ μg of residual hcDNA per 1.0×10¹³ vg; less than about 4 ng of rHCP per 1.0×10¹³ vg; pH of about 7.7-8.3; osmolality of about 390-430 mOsm/kg; less than about 600 particles that are 25 μm in size per container; less than about 6000 particles that are 10 μm in size per container; about 1.7×10¹³-5.3×10¹³ vg/mL genomic titer; infectious titer of about 3.9×10⁸-8.4×10¹⁰ IU per 1.0×10¹³ vg; total protein levels of about 100-300 μg per 1.0×10¹³ vg; relative potency of about 70-130% based on an in vitro cell-based assay, wherein the potency is relative to a reference standard and/or suitable control; less than about 5% empty capsid.

In some embodiments, a formulation or pharmaceutical composition comprises a unit dosage of rAAV viral vectors of about 2.4×10¹⁴ vg and one or more of the following release criteria: less than about 10%, less than about 8%, less than about 7%, or less than about 5% empty viral capsids; less than about 100 ng/mL host cell protein per 1×10¹³ vg/mL; less than about 5×10⁶ μg/mL, less than about 1×10⁶ μg/mL, less than about 7.5×10⁵ μg/mL, or less than 6.8×10⁵ μg/mL residual host cell DNA (hcDNA) per 1×10¹³ vg/mL; less than about 10 ng, less than about 8 ng, less than about 6 ng, or less than about 4 ng of residual host cell protein (rHCP) per 1.0×10¹³ vg/mL; at least about 50%, at least about 60%, at least about 70%, at least about 80%, at least about 90%, at least about 95%, or at least about 100% of rAAV viral vector genomes/mL that are functional; residual plasmid DNA of less than or equal to 1.7×10⁶ μg/mL per 1×10¹³ vg/mL, or 1×10⁵ μg/ml per 1×10¹³ vg/mL to 1.7×10⁶ μg/mL per 1×10¹³ vg/mL; benzonase concentrations of less than 0.2 ng per 1.0×10¹³ vg, less than 0.1 ng per 1.0×10¹³ vg, or less than 0.09 ng per 1.0×10¹³ vg; bovine serum albumin (BSA) concentrations of less than 0.5 ng per 1.0×10¹³ vg, less than 0.3 ng per 1.0×10¹³ vg, or less than 0.22 ng per 1.0×10¹³ vg; endotoxin levels of less than about 1 EU/mL per 1.0×10¹³ vg/mL, less than about 0.75 EU/mL per 1.0×10¹³ vg/mL, less than about 0.5 EU/mL per 1.0×10¹³ vg/mL, less than about 0.4 EU/mL per 1.0×10¹³ vg/mL, less than about 0.35 EU/mL per 1.0×10¹³ vg/mL, less than about 0.3 EU/mL per 1.0×10¹³ vg/mL, less than about 0.25 EU/mL per 1.0×10¹³ vg/mL, less than about 0.2 EU/mL per 1.0×10¹³ vg/mL, less than about 0.13 EU/mL per 1.0×10¹³ vg/mL, less than about 0.1 EU/mL per 1.0×10¹³ vg/mL, less than about 0.05 EU/mL per 1.0×10¹³ vg/mL, or less than about 0.02 EU/mL per 1.0×10¹³ vg/mL; concentrations of cesium less than 100 μg/g (ppm), less than 50 μg/g (ppm), or less than 30 μg/g (ppm); about 10-100 ppm, 15-90 ppm, or about 20-80 ppm of Poloxamer 188; fewer than 2000, fewer than 1500, fewer than 1000 or fewer than 600 particles that are 25 μm in size per container; fewer than 10000, fewer than 8000, fewer than 1000 or fewer than 6000 particles that are 0 μm in size per container; pH of between 7.5 to 8.5, between 7.6 to 8.4 or between 7.8 to 8.3; osmolality of between 330 to 490 mOsm/kg, between 360 to 460 mOsm/kg or between 390 to 430 mOsm/kg; infectious titer of about 1.0×10⁸-10.0×10¹⁰ IU per 1.0×10¹³ vg, about 2.5×10⁸-9.0×10¹⁰ IU per 1.0×10¹³ vg, or about 3.9×10⁸-8.4×10¹⁰ IU per 1.0×10¹³ vg; about 30-150%, about 60-140%, or about 70-130% relative potency based on an in vitro cell-based assay relative to a reference standard and/or suitable control; total protein levels of about 10-500 μg per 1.0×10¹³ vg, about 50-400 μg per 1.0×10¹³ vg, or about 100-300 μg per 1.0×10¹³ vg; an in vivo potency as determined by median survival in an SMNΔ7 mouse given at a 7.5×10¹³ vg/kg dose of greater than 15 days, greater than 20 days, greater than 22 days or greater than 24 days.

In some embodiments, a formulation or pharmaceutical composition comprises a unit dosage of rAAV viral vectors of about 2.4×10¹⁴ vg and one or more of the following release criteria: pH of about 7.7-8.3; osmolality of about 390-430 mOsm/kg; less than about 600 particles that are ≥25 μm in size per container; less than about 6000 particles that are ≥10 μm in size per container, about 1.7×10¹³-5.3×10¹³ vg/mL genomic titer; infectious titer of about 3.9×10⁸-8.4×10¹⁰ IU per 1.0×10¹³ vg; total protein levels of about 100-300 μg per 1.0×10¹³ vg; Pluronic F-68 content of about 20-80 ppm; relative potency of about 70-130% based on an in vitro cell-based assay, wherein the potency is relative to a reference standard and/or suitable control; in vivo potency characterized by median survival in a SMNΔ7 mouse model greater than or equal to 24 days at a dose of 7.5×10¹³ vg/kg; less than about 5% empty capsid; a total purity of greater than or equal to about 95%; less than or equal to about 0.13 EU/mL endotoxin.

In some embodiments, a formulation or pharmaceutical composition comprises a unit dosage of rAAV viral vectors of about 2.4×10¹⁴ vg and one or more of the following release criteria: less than about 0.09 ng of benzonase per 1.0×10¹³ vg; less than about 30 μg/g (ppm) of cesium; about 20-80 ppm of Poloxamer 188; less than about 0.22 ng of BSA per 1.0×10¹³ vg; less than about 6.8×10⁵ μg of residual plasmid DNA per 1.0×10¹³ vg; less than about 1.1×10⁵ μg of residual hcDNA per 1.0×10¹³ vg; less than about 4 ng of rHCP per 1.0×10¹³ vg; pH of about 7.7-8.3; osmolality of about 390-430 mOsm/kg; less than about 600 particles that are 25 μm in size per container; less than about 6000 particles that are 10 μm in size per container; about 1.7×10¹³-5.3×10¹³ vg/mL genomic titer; infectious titer of about 3.9×10⁸-8.4×10¹⁰ IU per 1.0×10¹³ vg; total protein levels of about 100-300 μg per 1.0×10¹³ vg; relative potency of about 70-130% based on an in vitro cell-based assay, wherein the potency is relative to a reference standard and/or suitable control; less than about 5% empty capsid.

The present disclosure is further illustrated by the following examples that should not be construed as limiting. The contents of all references, patents, and published patent applications cited throughout this application, as well as the figures, are incorporated herein by reference in their entirety for all purposes.

### EXAMPLES

### Pre-Clinical Example

The SMNΔ7 mouse is a suitable model to study gene transfer. Butchbach et al., “Abnormal motor phenotype in the SMNΔ7 mouse model of spinal muscular atrophy.” Neurobiology of disease, 27(2): 207-19. Injecting 5×10¹¹ viral genomes of scAAV9.CB.SMN into the facial vein on day 1 old mice rescues the SMNΔ7 mouse model. Foust et al., “Rescue of the spinal muscular atrophy phenotype in a mouse model by early postnatal delivery of SMN.” Nature biotechnology, 28(3): 271-4. Approximately 42±2% of lumbar spinal motor neurons were transduced in scAAV9.CB.SMN treated mice. SMN levels were increased as well, in brain, spinal cord, and muscle of scAAV9.CB.SMN-treated animals, compared to untreated SMA mice (although lower than WT controls). SMA animals treated with either scAAV9.CB.SMN or scAAV9.CB.GFP on P1 were assessed for their righting ability and were compared to Wild Type (WT) control mice and untreated mice. WT controls could right themselves quickly, whereas the SMN- and Green Fluorescent Protein (GFP)-treated SMA animals showed difficulty at P5. However, by P13, 90% of SMN-treated animals could right themselves compared with 20% of GFP-treated controls and 0% of untreated SMA animals. At P18, SMN-treated animals were larger than GFP-treated animals, but smaller than WT controls. Locomotive ability of the SMN-treated mice was nearly identical to WT controls, as assayed by open field testing and wheel running.

Survival of SMN-treated SMA animals compared with GFP-treated SMA animals was significantly improved. No GFP-treated control animals survived past P22 and had a median life span of 15.5 days. The weights of GFP mice peaked at P10 and then precipitously declined until death, while SMN mice showed a steady weight gain until around P40 with it stabilizing at 17 g (about half the weight of WT controls). The smaller size of corrected animals is likely related to the tropism and incomplete transduction of scAAV9, resulting in a ‘chimeric’ animal in which some cells were not transduced. Additionally, the smaller size suggests an embryonic role for SMN. Most remarkably, SMN-treated mice survived well past 250 days of age.

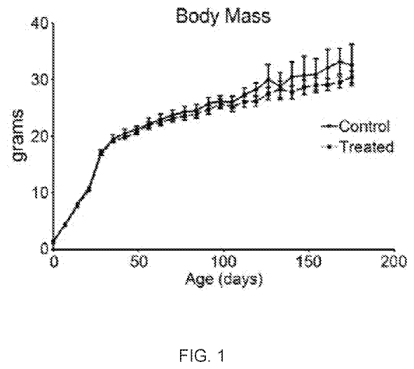

Toxicology biodistribution was also studied. In the non-Good Laboratory Practice (non-GLP) studies, 24 mice and 4 non-human primates (NHPs) were injected, by way of vascular delivery, with scAAV9.CB.SMN. To assess toxicity and safety scAAV9.CB.SMN was injected into P1 wild-type friend virus b-type (FVB) mice with either vehicle (PBS) (3 males/6 females) or 3.3×10¹⁴ vg/kg of scAAV9.CB.SMN (6 males/9 females) via the facial temporal vein. This dose was previously shown to be most efficacious in the SMNΔ7 mouse model of SMA16. P1 mice were used in anticipation of simulating potential clinical studies in infants, which is the planned population for the first-in-human clinical trial. All mice survived the injection procedure and the initial 24-hour observation period without any signs of distress or weight loss. Body mass was measured, and hands-on observations were performed weekly for the remainder of the study; neither revealed any difference between control and treated cohorts (FIG. 1).

At 60, 90 and 180 days post-injection, blood from the mice was collected for hematology studies and clinical chemistries assessment (ALT, AST, ALK Phos, creatinine, BUN, electrolytes, and CK). All were normal except for one variant at the 90-day time point. This difference appeared to be due to a technical problem relating to the site of blood draw, which differed from that of all other mice. For histopathology, 13 mice were necropsied at 120 days post-injection and 8 mice at 180 days. All organs were normal; in particular there was no inflammation seen in any section from any organ (heart, liver, kidney, muscle, gonads, brain, lung, lymph nodes, and intestines).

In the safety study for the four male Cynomolgus Macaques, subjects were injected at 90 days of age to closely mimic the likely age of administration of treatment in SMA Type I infants. The scAAV9.CB.SMN vector was administered one time by catheterization of the saphenous vein with a dose of 6.7×10¹³/kg, which corresponds to the lowest dose tested for which SMN-07 mice showed a significant increase of survival. Animals were followed for six months until they were sacrificed at approximately 9 months of age. No adverse effects were seen, and all clinical chemistries were normal. T-cell immune response was tested using ELISpot in peripheral blood mononuclear cells (PBMCs), and all were negative at 6 months post injection.

In these non-GLP studies, serum chemistry and hematology studies were unremarkable as was the histopathology assessment. The NHP subjects mounted appropriate immune responses to capsid (but not to transgene), with very high transgene expression persisting at 6 months post-injection. These studies provide strong evidence that systemically-delivered scAAV9.CB.SMN is safe and well tolerated, even at the high doses used for penetration of the blood-brain barrier. Foust et al. Nat. Biotechnol., 28(3), pp. 271-274 (2010).

When newborn FVB mice were given a single intravenous injection of scAAV9.CB.SMN at levels up to 3.3×10¹⁴ vg/kg on Day 1, there was neither test article-related mortality nor evidence of toxicity seen at time points up to 24 weeks after administration. Treatment-related decreases in mean body weight and mean body weight gain, as well as lower activated partial thromboplastin time (APTT) values, were mild effects of treatment, but did not yield toxicity.

Activity of the scAAV9.CB.SMN was demonstrated by the bio distribution and the presence of a specific transgene ribonucleic acid (RNA) expression in brain and spinal cord, the main targeted therapeutic tissues. Low levels of antibodies to the AAV9 capsid were found after 12 and 24 weeks in males and females given 3.3×10¹⁴ vg/kg (Group 3). No alteration was observed in clinical pathology and histopathology analyses. Based on these results, the no observable adverse effect level (NOAEL) of scAAV9.CB.SMN in newborn male and female mice is considered to be 3.3×10¹⁴ vg/kg.

In these studies, scAAV9.CB.SMN intrathecal administration to the CSF was safe and well tolerated in mice (through Week 12) and macaques (up to 14 months post injection). CSF delivery in mice likely reduced periphery exposure of scAAV9.CB.SMN and qualitative polymerase chain reaction (qPCR) results indicate transgene expression was higher in cervical and lumbar regions compared to the thoracic region. Monkeys maintained in the Trendelenburg position for 5 minutes at injection and were confirmed seronegative for anti-AAV9 antibodies prior to injection. All non-human primates were highly positive for AAV9 antibodies up to 6 months post injection. No cytotoxic T-lymphocyte response to either AAV9 capsid or SMN transgene was observed for 6 months post injection. No tissue degradation or reactive response in the brain or spinal cord was observed.

In pivotal Good Laboratory Practice (GLP) compliant 3-month mouse toxicology studies, the main target organs of toxicity were the heart and liver. Following IV infusion in the mouse, vector and transgene were widely distributed with the highest expression generally observed in heart and liver, and substantial expression in the brain and spinal cord. AVXS-101-related findings in the ventricles of the heart were comprised of dose-related inflammation, edema and fibrosis, and in the atrium, inflammation and thrombosis. Liver findings were comprised on hepatocellular hypertrophy, Kupffer cell activation, and scattered hepatocellular necrosis. A NOAEL was not identified for AVXS-101-related heart and liver findings in the mouse, and the Maximum Tolerated Dose was defined as 1.5×10¹⁴ vg/kg, providing a safety margin of approximately 1.4-fold relative to the recommended therapeutic dose of 1.1×10¹⁴ vg/kg. The translatability of the observed findings in mice to primates is not known at this time.

These data support moving forward to clinical trials.

To determine whether CSF delivery can reduce the transduction of peripheral organs compared to the intravenous (IV) injections, a detailed bio distribution analysis was performed on the tissue of the nonhuman primates that were placed head down in the Trendelenburg position for either 5 or 10 minutes (n=5). These animals were selected over the nonhuman primates that were not placed head down because the treatment highly improved distribution in the spinal cord and brain, favoring this approach for clinical trials. Two weeks post-injection, the cynomolgus macaques were sacrificed and various tissues were collected to perform detailed Deoxyribonucleic Acid (DNA) and RNA bio distribution analyses. scAAV9.CBA.GFP was lower in most peripheral tissues except spleen and liver compared to the high levels in brain and spinal cord. These findings are in line with previous reports from other groups. Dirren et al., “Intracerebroventricular injection of adeno-associated virus 6 and 9 vectors for cell type specific transgene expression in the spinal cord.” Hum Gene Ther 25: 109-120; Gray et al., “Global CNS gene delivery and evasion of anti-AAV-neutralizing antibodies by intrathecal AAV administration in non-human primates.” Gene Ther 20: 450-459. In the skeletal muscles and the CNS, there is a strong correlation between DNA and RNA levels, while in soft tissues and glands, RNA levels are generally lower than expected for the viral genomes detected. In particular, testes, intestines, and spleen show a 1,000 times fewer RNA molecules than DNA. Despite the detection of AAV in peripheral organs, there was a significant decrease in the amount of vector detected peripherally compared to systemic injection. Dirren et al.; Gray et al. Additionally, similar observations were made when comparing mice that were injected either intravenously or intracerebroventricularly at P1 24 weeks post-treatment. Thus, CSF delivery is adding a significant potential safety component to future clinical trials with AVXS-101.

In some embodiments, Trendelenburg positioning improves CSF delivery. Dosing and efficacy of scAAV9-SMN was evaluated in SMA mice and non-human primates, delivered directly to the CSF via single injection. Widespread transgene expression was observed throughout the spinal cord in mice and nonhuman primates when using a 10 times lower dose compared to the IV application. In nonhuman primates, lower doses than in mice can be used for similar motor neuron targeting efficiency. The transduction efficacy was found to be further improved when subjects were kept in the Trendelenburg position to facilitate spreading of the vector. Meyer et al., “Improving single injection CSF delivery of AAV9-mediated gene therapy for SMA: a dose-response study in mice and nonhuman primates.” Molecular therapy: the journal of the American Society of Gene Therapy 23, 477-487. Tilting the animals significantly improved transduction in the thoracic and cervical region of the spinal cord, as demonstrated by immunofluorescence and quantification of GFP/ChAT double positive motor neurons. Tilting for 10 minutes was sufficient to increase motor neuron transduction to 55, 62, and 80% in the cervical, thoracic, and lumbar region respectively, which implies major benefits for patients according to the rescue observed in the mouse model. The motor neuron counts tightly correlated with GFP transcript quantification in each of the spinal cord segments.

### Example 1—Clinical Trial Protocol

A Phase 1, open-label, single-dose administration clinical trial is performed on infants and children with a genetic diagnosis consistent with SMA, bi-allelic deletion of SMN1 and 3 copies of SMN2 without the genetic modifier who are able to sit but cannot stand or walk at the time of study entry. Patients receive AVXS-101 in a dose comparison safety study of up to three (3) potential therapeutic doses as described below. Patients are stratified in two groups, those ≥6 months and <24 months of age at time of dosing and those ≥24 months and <60 months of age at time of dosing. At least fifteen (15) patients ≥6 months and <24 months are enrolled and twelve (12) patients ≥24 and <60 months are enrolled.

The first cohort enrolls three (3) patients (Cohort 1) ≥6 months and <24 months of age who will receive administration of 6.0×10¹³ vg of AVXS-101 (Dose A). There are at least a four (4) week interval between the dosing of each patient within the cohort. The investigators confer with the Data Safety Monitoring Board (DSMB) on all Grade III or higher AEs within 48 hours of awareness that are possibly, probably, or definitely related to the study agent before continuing enrollment. Following enrollment of the first three patients and based upon the available safety data a decision is made whether to: a) stop due to toxicity, or b) proceed to Cohort 2 using Dose B.

For Dose B, three (3) patients <60 months of age are enrolled to receive administration of 1.2×10¹⁴ vg of AVXS-101 (Dose B). Again, there is at least a 4-week interval between dosing of the three patients within the cohort. Based on the available safety data from the three Cohort 2 patients and all of the Cohort 1 patients, further 4-week intervals between patients dosing may be unnecessary. The investigators confer with the DSMB on all Grade III or higher AEs within 48 hours that are possibly, probably, or definitely related to the study agent before continuing enrollment. Following enrollment of the first six (6) patients and based upon available safety data, a decision is made whether to a) stop due to toxicity, or b) continue to enroll an additional 21 patients until twelve (12) patients ≥6 months and <24 months and twelve (12) patients ≥24 months and <60 months have received Dose B.

Based upon an ongoing assessment of safety and efficacy data from patients treated with the 1.2×10¹⁴ vg dose, testing of a third dose (Dose C), is considered. Three (3) patients <60 months of age receive Dose C, which will be up to 2.4×10¹⁴ vg administered intrathecally. There is again a four-week interval between dosing of the first three patients receiving Dose C, as in Cohorts 1 and 2. Following enrollment of the first three (3) Dose C patients and based upon available safety data, a decision is made whether to: a) stop due to toxicity, or b) continue to enroll an additional 21 patients until there are a total of twelve (12) patients >6 months and <24 months and twelve (12) patients ≥24 and <60 months that have received Dose C.

Selection of the appropriate dose and justification for testing Dose C may be supported by ongoing safety and efficacy reviews of clinical findings from the patients receiving Dose B (1.2×10¹⁴ vg). The selected dose is up to 2.4×10¹⁴ vg delivered intrathecally. Doses up to 1.1×10¹⁴ vg/kg have been safely administered systemically (intravenously) to children weighing up to 8.4 kg (total dose 9.24×10¹⁴ vg). In addition, in preclinical studies, the intrathecal administration of scAAV9.CB.SMN was safe and well tolerated up to 14 months post injection in large non-human primates at a dose of 2×10¹³ vg/kg.

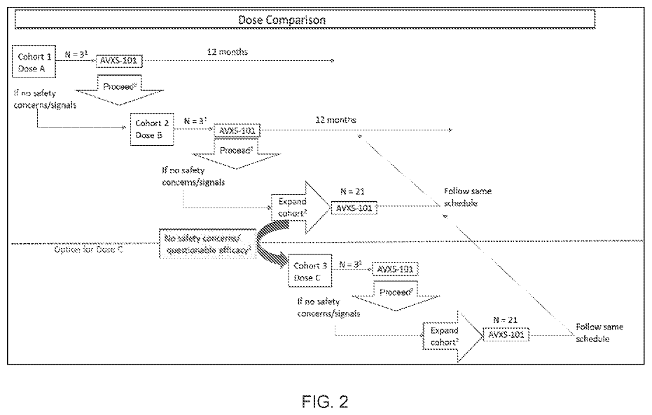

The overall study design is summarized in FIG. 2.

Safety is assessed through monitoring adverse event (AE) reports and concomitant medication usage, and by conducting physical examinations, vital sign assessments, cardiovascular evaluations, and laboratory evaluations. Patients are observed at the hospital for 48 hours post intrathecal injection. Patients return for follow up visits on Days 7, 14, 21, and 30. Patients return monthly thereafter, following the Day 30 visit, for 12 months from dose administration. Upon study completion, study patients are asked to enroll in a vital long-term follow-up study examining the lasting safety of AVXS-101 up to 15 years.

### Number of Patients

At least 27 patients are enrolled; up to 51 patients may be enrolled if escalation to Dose C is determined necessary.

### Treatment Assignment

This is an open-label comparative single-dose study. Treatment is assigned in accord with the dose escalation schedule specified herein.

### Dose Adjustment Criteria

The study investigates a one-time intrathecal injection of AVXS-101.

### Criteria for Study Termination

An independent Data Safety Monitoring Board (DSMB) and medical monitor monitors safety data on a continual basis throughout the trial. The DSMB can recommend early termination of the trial for reasons of safety. Study enrollment is halted by the investigators if any patient experiences a Grade III, or higher AE toxicity that is unanticipated and possibly, probably, or definitely related to the study product that presents with clinical symptoms and requires medical treatment. This includes any patient death, important clinical laboratory finding, or any severe local complication in the injected area related to administration of the study agent.

The trial may be terminated if the DSMB recommends an early termination of the study for safety reasons. The trial may also be terminated by recommendation of the Regulatory Authority. Lastly, the trial may also be terminated if patients develop unacceptable levels of toxicity, defined as the occurrence of any unanticipated CTCAE Grade 3 or higher AE/toxicity that is possibly, probably, or definitely related to gene replacement therapy, and is associated with clinical symptoms and/or requires medical treatment.

### Patient Inclusion Criteria

Patients meet all of the following inclusion criteria:

1. Patients ≥6 months of age and up to 60 months (1800 days) of age at time of dosing following diagnostic confirmation during screening period by genotype who demonstrate the ability to sit unassisted for 10 or more seconds but cannot stand or walk
Diagnostic confirmation by genotype includes lab documentation of homozygous absence of SMN1 exon 7; with exactly three copies of SMN2.
   2. Negative gene testing for SMN2 gene modifier mutation (c.859G>C).
   3. Onset of clinical signs and symptoms consistent with SMA at <12 months of age.
   4. Able to sit independently and not standing or walking independently. Definition of sitting independently is defined by the World Health Organization (WHO)-MGRS criteria of being able to sit up unsupported with head erect for at least 10 seconds. Child should not use arms or hands to balance body or support position (Wijnhoven 2004).
   5. Meet age-appropriate institutional criteria for use of anesthesia and sedation, as determined necessary by the investigator.
   6. Be up-to-date on childhood vaccines. Seasonal vaccinations that include palivizumab prophylaxis (also known as Synagis) to prevent respiratory syncytial virus (RSV) infections are also recommended in accordance with American Academy of Pediatrics (AAP 2009).
   7. Parent(s)/legal guardian(s) willing and able to complete the informed consent process.

### Patient Exclusion Criteria

Patients must not meet any of the following exclusion criteria:

1. Current or historical ability to stand or walk independently.
2. Contraindications for spinal tap procedure or administration of intrathecal therapy (e.g., spina bifida, meningitis, impairment, or clotting abnormalities, or obstructive spinal hardware preventing effective access to CSF space) or presence of an implanted shunt for the drainage of CSF or an implanted CNS catheter.
3. Severe contractures as determined by designated Physical Therapist(s) at screening that interfere with either the ability to attain/demonstrate functional measures (e.g., standing, walking) or interferes with ability to receive IT dosing 10. Severe scoliosis (defined as ≥50° curvature of spine) evident on X-ray examination.
4. Previous, planned or expected scoliosis repair surgery/procedure within 1 year of dose administration.
5. Use of invasive ventilatory support (tracheotomy with positive pressure) or pulse oximetry <95% saturation at screening while the patient is awake, or for high altitudes >1000 m, oxygen saturation <92% while the patient is awake
Pulse oximetry saturation must not decrease ≥four (4) percentage points between screening and highest value on day of dosing.
   6. Use or requirement of non-invasive ventilatory support for 12 or more hours daily in the two weeks prior to dosing.
   7. Medical necessity for a gastric feeding tube, where the majority of feedings are given by non-oral methods (i.e., nasogastric tube or nasojejunal tube) or patients whose weight-for-age falls below the 3rd percentile based on WHO Child Growth Standards (Onis 2006). Placement of a permanent gastrostomy prior to screening is not an exclusion.
   8. Active viral infection (includes HIV or serology positive for hepatitis B or C, or Zika virus).
   9. Serious non-respiratory tract illness requiring systemic treatment and/or hospitalization within two (2) weeks prior to study entry.
   10. Respiratory infection requiring medical attention, medical intervention or increase in supportive care of any manner within four (4) weeks prior to study entry.
   11. Severe non-pulmonary/respiratory tract infection (e.g., pyelonephritis, or meningitis) within four (4) weeks before study dosing or concomitant illness that in the opinion of the PI creates unnecessary risks for gene transfer such as:
Major renal or hepatic impairment
Known seizure disorder
Diabetes mellitus
Idiopathic hypocalciuria
Symptomatic cardiomyopathy
   12. History of bacterial meningitis or brain or spinal cord disease, including tumors, or abnormalities by MRI or CT that would interfere with the LP procedures or CSF circulation.
   13. Known allergy or hypersensitivity to prednisolone or other glucocorticosteroids or their excipients.
   14. Known allergy or hypersensitivity to iodine or iodine-containing products.
   15. Concomitant use of any of the following: drugs for treatment of myopathy or neuropathy, agents used to treat diabetes mellitus, or ongoing immunosuppressive therapy, plasmapheresis, immunomodulators such as adalimumab, or immunosuppressive therapy within 3 months of study dosing (e.g., corticosteroids, cyclosporine, tacrolimus, methotrexate, cyclophosphamide, intravenous immunoglobulin, rituximab).
   16. Inability to withhold use of laxatives or diuretics in the 24 hours prior to dose administration.
   17. Anti-AAV9 antibody titers >1:50 as determined by ELISA binding immunoassay
Should a potential patient demonstrate anti AAV9 antibody titer >1:50, he or she may receive retesting within 30 days of the screening period and will be eligible to participate if the anti AAV9 antibody titer upon retesting is ≤1:50.
   18. Abnormal laboratory values considered to be clinically significant (INR>1.4), GGT>3×ULN, Bilirubin≥3.0 mg/dL, Creatinine≥1.0 mg/dL, Hgb<8 or >18 g/DI; WBC>20,000 per cmm) prior to study dosing.
   19. Participation in recent SMA treatment clinical trial or receipt of an investigational or approved compound product or therapy received with the intent to treat SMA (e.g., valproic acid, nusinersen) at any time prior to screening for this study
Oral beta agonists must be discontinued 30 days prior to dosing
Inhaled albuterol specifically prescribed for the purposes of respiratory (bronchodilator) management is acceptable and not a contraindication at any time prior to screening for this study.
   20. Expectation of major surgical procedures during the 1-year study assessment period (e.g., spinal surgery or tracheostomy).
   21. Inability or unwillingness to comply with study procedures or inability to travel for repeat visits.
   22. Unwillingness to keep study results/observations confidential or to refrain from posting confidential study results/observations on social media sites.
   23. Refusal to sign consent form.

### Patient Withdrawal Criteria and Discontinuation

Patients may be discontinued from the study if they develop unacceptable levels of toxicity, defined as the occurrence of any unanticipated CTCAE Grade 3 or higher Adverse Event/toxicity that is possibly, probably, or definitely related to the gene replacement therapy, and is associated with clinical symptoms and/or requires medical treatment. Patients are withdrawn if they die, in which case autopsies will be requested of any patients, with the exception of untreated patients, that expire following participation in a gene transfer study. Patients may also be withdrawn if they fail to comply with protocol-required visits or study procedures for 3 or more consecutive visits that are not rescheduled, unless due to hospitalization. Patients whose parent(s) or legal guardian(s) withdraws consent are also withdrawn from the study. Finally, patients may be withdrawn at the discretion of the investigator. Early termination procedures should be completed within 14 days for any patient who prematurely discontinues the study for any reason.

### Description of Study Product

The biological product is a non-replicating recombinant self-complementary adeno-associated virus serotype 9 (AAV9) containing the cDNA of the human SMN gene under the control of the cytomegalovirus (CMV) enhancer/chicken-β-actin-hybrid promoter (CB). The AAV inverted terminal repeat (ITR) has been modified to promote intramolecular annealing of the transgene, thus forming a double-stranded transgene ready for transcription. This modified ITR, termed a “self-complementary” (sc) ITR, has been shown to significantly increase the speed of which the transgene is transcribed, and the resulting protein is produced. Cells transduced with AVXS-101 (scAAV9.CB.hSMN) express the human SMN protein.

TABLE 3
Investigational Product

| | Investigational Product |
|---|---|
| Product Name | AVXS-101 |
| Unit Dose | 6.0 × 10¹³ vg (Dose A) |
| | 1.2 × 10¹⁴ vg (Dose B) |
| | No more than 2.4 × 10¹⁴ vg (Dose C) |
| Route of Administration | Intrathecal Injection |
| Physical Description | Once thawed, AVXS-101 is a clear to |
| | slightly opaque, colorless to faint white |
| | solution, free of visible particulates |

### Prior and Concomitant Medications

Prior and concomitant medications are captured in an electronic Case Report Form (eCRF) from two weeks prior to study dosing until the End of Study visit.

### Prophylactic Administration of Prednisolone

An antigen specific T-cell response to the AAV vector was observed in the ongoing Phase 1 clinical study investigating AVXS-101 treatment via intravenous infusion. This is an expected response between 2-4 weeks following gene transfer. One possible consequence to such antigen specific T-cell response is clearance of the transduced cells and loss of transgene expression.

In an attempt to dampen the host immune response to the AAV based therapy, patients receive prophylactic prednisolone (glucocorticoid) (approximately 1 mg/kg/day) 24 hours prior to AVXS-101 dosing. Treatment continues for approximately 30 days in accord with the following treatment guideline:
Until at least 30 days post-infusion: 1 mg/kg/day
Weeks 5 and 6: 0.5 mg/kg/day
Weeks 7 and 8: 0.25 mg/kg/day
Week 9: prednisolone discontinued

If the aspartate aminotransferase (AST) or alanine aminotransferase (ALT) values are >2×upper limit of normal (ULN), or if T-cell response is ≥100 SFC/10⁶ PBMCs after 30 days of treatment, the dose of prednisolone is maintained until the AST and ALT values decrease below threshold. If T-cell response continues past Day 60, investigator discretion should be used considering risk benefit for maintaining prednisolone. Variance from these recommendations is at the discretion of the investigator based on potential safety issues for each patient.

### Prohibited Medications

Concomitant use of any of the following medications is prohibited:
Drugs for treatment of myopathy or neuropathy
Agents used to treat diabetes mellitus
Therapy received with the intent to treat SMA (e.g., valproic acid, nusinersen).
   Oral beta-agonists must be discontinued at least 30 days prior to gene therapy dosing.
   Inhaled beta agonists may be used to treat respiratory complications of SMA provided such medications are dosed at clinically appropriate levels
Ongoing immunosuppressive therapy, plasmapheresis, immunomodulators such as adalimumab, or immunosuppressive therapy within 3 months of starting the trial (e.g., corticosteroids, cyclosporine, tacrolimus, methotrexate, cyclophosphamide, intravenous immunoglobulin, rituximab)

Corticosteroid usage following completion of the prednisolone taper is permissible at the discretion of the managing physician as part of routine clinical management. The use of prednisone in such circumstances should be documented appropriately as a concomitant medication, and the event precipitating its usage should be appropriately documented as an AE.

Should the use of corticosteroids (aside from inhaled corticosteroids for bronchospasm) be considered as part of care during the course of the prednisolone taper, this medical management should be discussed with the sponsor medical monitor, who is responsible for any indicated medication adjustments related to the taper.

### Treatment Compliance

AVXS-101 is administered as a one-time intrathecal injection.

### Randomization and Blinding

This is an open-label study.

### Study Product Dose and Dose Justification

Patients receive a one-time dose of AVXS-101 6.0×10¹³ vg, 1.2×10¹⁴ vg, or a third dose of up to 2.4×10¹⁴ vg, if determined necessary via intrathecal injection. The delivery directly into the CSF via intrathecal injection allows for reduction of the amount of viral vector approximately by a factor of ten with equal distribution and efficacy throughout the CNS, reducing viral vector loads and further optimizing. Selection of the appropriate dose and justification for studying all dose escalations are further supported by ongoing safety and efficacy reviews of clinical findings from the patients receiving previous doses as described. The highest selected dose is up to 2.4×10¹⁴ vg delivered intrathecally. Doses up to 1.1×10¹⁴ vg/kg have been safely administered systemically (intravenously) to children weighing up to 8.4 kg (total dose 9.24×10¹⁴ vg). In addition, in preclinical studies, the intrathecal administration of scAAV9.CB.SMN was safe and well tolerated up to 14 months post injection in large non-human primates at a dose of 2×10¹³ vg/kg.

### Study Product Preparation

Preparation of AVXS-101 is done aseptically under sterile conditions by a pharmacist.

AVXS-101 is pre-mixed with an appropriate contrast medium approved and labeled for pediatric use for radiographic monitoring of the injection via lumbar intrathecal injection. The total volume of AVXS-101+contrast medium does not exceed 8 m L.

The dose-delivery vessel is delivered to the designated pediatric intensive care unit (PICU) patient room or other appropriate setting (e.g., interventional suite, operating room, dedicated procedure room) with immediate access to acute critical care management.

Patients receive AVXS-101 intrathecal injection under sterile conditions in a PICU patient room or other appropriate setting (e.g., interventional suite, operating room, dedicated procedure room) with immediate access to acute critical care management. Patients are admitted, and vitals monitored every 15 (+/−5) minutes for four hours and every hour (+/−15 minutes) for 24 hours following the AVXS-101 dosing procedure.

Sites are instructed to use an atraumatic needle inserted with the bevel parallel to the dura fibers; this has been shown to considerably reduce damage to the dura and consequently decrease the risk for cerebrospinal fluid leak after lumbar puncture including in children. Ebinger et al., “Headache and backache after lumbar puncture in children and adolescents: a prospective study.” Pediatrics, 113:1588-1592; Kiechl-Kohlendorfer et al., “Cerebrospinal fluid leakage after lumbar puncture in neonates: incidence and sonographic appearance.” Am J Roentgenol, 181:231-234.

Sedation/anesthesia is required for all patients receiving AVXS-101. Method and medications are at the discretion of the local anesthesiologist but should incorporate a sufficient degree of sedation or anxiolysis to ensure analgesia and lack of movement for the procedure and post-procedure Trendelenburg positioning placement. Patients are placed in the Trendelenburg position, tilted head-down at 30° for 15 minutes following administration of vector to enhance distribution to cervical and brain regions.

AVXS-101 is administered by an investigator or interventional radiologist or other appropriately trained and experienced physician under sterile conditions with fluoroscopic/radiographic guidance as per institutional guidelines. Patients are placed in the lateral decubitus position and a catheter with stylet is inserted by a lumbar puncture into the L3-L4 or L4-L5 interspinous space into the subarachnoid space. Subarachnoid cannulation is confirmed with the flow of clear cerebrospinal fluid (CSF) from the catheter. Approximately four (4) mL CSF is removed for Dose A and Dose B, a volume of CSF closely approximating the volume of AVXS-101 plus contrast injected (up to seven (7) mL) is removed for Dose C and disposed of as per institutional guidelines. AVXS-101 in the pre-mixed contrast solution is injected directly into the subarachnoid space. Flushing of the injection needles with 0.5 mL saline is allowed as per institutional standards/guidelines.

### Post-Administration Procedures

Following AVXS-101 administration patients return to a designated PICU bed, or other appropriate setting, with close monitoring of vital signs. Concomitant medications and all AEs/serious AEs are also monitored and documented following dosing procedures.

Patients are kept in the PICU patient room or other appropriate setting (e.g., interventional suite, operating room, dedicated procedure room) with immediate access to acute critical care management for 48 hours for closer monitoring of mental status. During the inpatient stay, personnel are required to follow appropriate safety precautions as per institutional standards for infection control; standards should require personal protective equipment (PPE) such as gowns, gloves, masks, glasses, and closed-toe shoes. Patients' families are provided standardized, IRB-approved handouts regarding monitoring for mental status changes which includes monitoring for fever, irritability, neck pain, light sensitivity and vomiting. Patients may be discharged from the hospital when the following criteria are met:
Afebrile
Absence of hypersensitivity reactions
Absence of meningismus
Absence of abnormal laboratory values suggestive of possible CNS infection or complication

### Dose Escalation

There is a 4-week dosing interval between all patients within Cohort 1 to allow review of the safety analysis from six-time points (Days 1, 2, 7, 14, 21, 30) prior to dosing of the next patient.

The investigators confer with the DSMB on all Grade III or higher AEs within 48 hours of awareness that are possibly, probably, or definitely related to the study agent before continuing enrollment. Following enrollment of the first three (3) patients ≥6 months and <24 months of age at the time of dosing and based upon the available safety data a decision is made whether to: a) stop due to toxicity, or b) proceed to Cohort 2 using Dose B.

For Dose B, there is at least a 4-week interval between dosing of the first three (3) patients <60 months of age at the time of dosing within the cohort. Based on the available safety data from the first three (3) Cohort 2 patients and all of the Cohort 1 patients, further 4-week intervals between patients dosing may be unnecessary. The investigators confer with the DSMB on all Grade III or higher AEs within 48 hours that are possibly, probably, or definitely related to the study agent before continuing enrollment. Following enrollment of the first six (6) patients and based upon available safety data a decision is made whether to a) stop due to toxicity or b) continue to enroll an additional 21 patients until twelve (12) patients ≥6 months and <24 months of age at time of dosing and twelve (12) patients >24<60 months of age at time of dosing have received Dose B.

Based upon an ongoing assessment of safety and efficacy data from patients treated with the 1.2×10¹⁴ vg dose, testing of a third dose (Dose C) may be considered. Three (3) patients <60 months of age receive Dose C which will be up to 2.4×10¹⁴ vg administered intrathecally. There will again be a four-week interval between dosing of the first three patients receiving Dose C, as in Cohorts 1 and 2. Following enrollment of the first three (3) Dose C patients and based upon available safety data a decision is made whether to: a) stop dosing Dose C due to safety concern, or b) continue to enroll an additional 21 patients until there are a total of twelve (12) patients ≥6 months and <24 months and twelve (12) patients ≥24 and <60 months that have received Dose C.

### Physical Therapy Assessments: Hammersmith Functional Motor Scale—Expanded

The Hammersmith Functional Motor Scale-Expanded was devised for use in children with spinal muscular atrophy Type 2 and Type 3, to give objective information on motor ability and clinical progression.

The Hammersmith Functional Motor Scale-Expanded is administered by a physical therapist in accord with Table 4 within 30 days of dosing and monthly through twelve (12) months for all patients ≥24 months of age. Patients <24 months of age at time of dosing begin having Hammersmith Functional Motor Scale-Expanded assessments at such time that 24 months of age is reached. The Hammersmith Functional Motor Scale-Expanded sessions are videotaped.

### Physical Therapy Assessments: Bayley Scales of Infant and Toddler Development®

Bayley Scales of Infant and Toddler Development®, Third Edition is a standardized, norm-referenced infant assessment. The gross and fine motor subtests were completed within 30 days before dosing at baseline and then monthly through Month 12. Bayley Scales® assessments are videotaped.

### Physical Therapy Assessments: Motor Milestone Development Survey

The achievement of significant motor milestones are assessed by the physical therapist using a standard Motor Milestone Development Survey shown in Table 2 with definitions of each milestone driven by the Bayley Scales® (see Physical Therapy Manual). The physical therapist records whether the patient has attained each of the milestones on the Motor Milestone Development Survey in accordance with Table 4. Once observed, a motor milestone is considered attained. The date of attainment of each motor milestone is determined by the date of the visit in which the milestone is observed. During the Screening visit, the physical therapist completes an assessment of baseline milestone achievement in accordance with Table 4; this assessment is recorded on video and the findings documented. As the Bayley Scales® do not necessarily require the child to repeat previously attained milestones, each milestone may be captured on video. Development milestone assessment sessions are documented.

TABLE 4
Schedule of Assessments

| | Study Interval | | | | | | | | | |
|---|---|---|---|---|---|---|---|---|---|---|
| | Baseline | Vector (AVXS-101) | | | | | | | | |
| | Screening | Injection (Inpatient) | | | | | | | | |
| | Visit # | | | | | | | | | |
| | | | | | | | | | Monthly | Month 12 |
| | 1 | 2 | | | 3 | 4 | 5 | 6 | (7-16) | or EOS (17) |
| | # Days/Month in Study | | | | | | | | | |
| | | | | | | | | | Through | |
| | −60 to −2 | −1 | 1 | 2-3 | 7 | 14 | 21 | 30 | month 11 | Month 12 |
| Window | | | | | +/−2 | | | +/−7 | | +/−7 |
| Informed Consent | X | | | | | | | | | |
| Spinal X-ray | X | | | | | | | | | |
| Demographics/Medical History | X | | X | X | X | X | X | X | X | X |
| Physical Exam | X | | X | X | X | X | X | X | X | X |
| Vitals/Weight/Length/Height | X | | X | X | X | X | X | X | X | X |
| Pulse Oximetry | | | X | X | X | X | X | X | X | X |
| Pulmonary Exam | X | | | | | | | X | X | X |
| 12-Lead ECG | X | | X | X | | | | | X | X |
| 12-Lead Holter Monitor | X | X | X | X | | | | X | X | X |
| Echocardiogram | X | | | | | | | | X | X |
| Capillary Blood Gas | | X | | X | | | | | | |
| HFMS-Expanded (with video) | X | | | | | | | X | X | X |
| Bayley ®-III (with video) | X | | | | | | | X | X | X |
| Motor Milestone Development | X | | | | | | | X | X | X |
| Survey (with video) | | | | | | | | | | |
| Hematology/Chemistry | X | X | | X | X | X | X | X | X | X |
| CK-MB | X | | | | X | | | X | X | X |
| Troponin I | X | | | | X | | | X | X | X |
| Coagulation | X | X | | X | X | X | X | X | X | X |
| Urinalysis | X | X | | X | X | X | X | X | X | X |
| Virus Serology | X | | | | | | | | | |
| Blood for diagnostic confirmation | X | | | | | | | | | |
| testing | | | | | | | | | | |
| Saliva, Urine, and Stool Samples | | X | | X | X | X | | X | | |
| (for viral shedding) | | | | | | | | | | |
| Baseline screening of Mother (anti- | X | | | | | | | | | |
| AAV9 Ab) | | | | | | | | | | |
| Immunology Labs (anti-AAV9/SMN) | X | | | | X | X | X | X | | |
| Immunology Labs (IFN-γ T-cells) | | | | | X | X | X | X | | |
| Prednisolone dosing | | X | X | X | X | X | X | X | | |
| Study Product administration with | | | X | | | | | | | |
| fluoroscopic/radiographic guidance | | | | | | | | | | |
| Photograph injection site | | | X | | X | X | X | X | | |
| Adverse Events | X | X | X | X | X | X | X | X | X | X |

| | |
|---|---|
| Prior and Concomitant Medications | To be collected from 2 weeks before study dosing until final study visit |

### Video Evidence

Physical therapy assessments at each study visit are videotaped in an effort to produce compelling, demonstrable, documented evidence of efficacy, as determined by changes in functional abilities. Parent(s)/legal guardian(s) may also share home videos demonstrating achievement of functional abilities with the study site.

Videos are provided to an independent, centralized reviewer for unbiased assessment of milestone achievement. The independent reviewer uses the Motor Milestone Development Survey to document whether the video displays evidence of having achieved each motor milestone. The date of motor milestone achievement is computed as the earliest of the video dates in which achievement of the milestone has been demonstrated.

### Other Clinical Assessments: Demographic/Medical History

Patient demographics and medical history information are collected at baseline and captured in a Case Report Form (CRF). Medical history throughout the study is collected at each visit. Medical History information includes, but is not limited to: familial history of spinal muscular atrophy including affected siblings or parent carriers, gestational age at birth, length/height/head circumference at birth, hospitalization information from time of birth including number, duration, and reason for hospitalizations including ICD-10 codes if available, historical ventilatory support, if any, and historical feeding support, if any.

### Other Clinical Assessments: Vital Signs

Vital signs include blood pressure, respiratory rate, pulse, and axillary temperature within 30 days of dosing and at the time points specified in Table 4. Vitals including pulse oximetry and heart rate are continuously monitored and recorded by a team member during the injection. At Visit 2, vitals including blood pressure, respiratory rate, pulse axillary temperature, pulse oximetry and heart rate are monitored and recorded every 15 minutes (+/−5 minutes) for four hours and every hour (+/−15 minutes) for 24 hours following the AVXS-101 dosing procedure.

### Other Clinical Assessments: Weight and Length/Height

Weight and length and/or height, as appropriate are measured as per the time points specified in Table 4.

### Other Clinical Assessments: Physical Examination

Physical examination includes review of the following systems: head, eyes, ears, nose and throat (HEENT), lungs/thorax, cardiovascular, abdomen, musculoskeletal, neurologic, dermatologic, lymphatic, and genitourinary. The head circumference is measured with each physical examination. To measure head circumference, the examiner securely wraps a flexible measuring tape around the circumference of the head, above the eyebrows over the broadest part of the forehead, above the ears, and over the most prominent part of the occiput. The measurement should be taken 3 times, and the largest measurement should be recorded to an accuracy of 0.1 cm. Baseline physical examinations are completed within 30 days of dosing, and in accord with the time points specified in Table 4.

### Other Clinical Assessments: Vaccination Recommendations

Patients are encouraged to follow all routinely scheduled immunizations as recommended by the Center for Disease Control (CDC). Seasonal vaccinations that include palivizumab prophylaxis (also known as Synagis) to prevent respiratory syncytial virus (RSV) infections are also recommended in accordance with American Academy of Pediatrics (AAP 2009).

### Other Clinical Assessments: 12-Lead Electrocardiogram (ECG)

A 12-lead ECG is performed at screening/baseline, Day 1, Day 2, Day 3, Month 3, Month 6, Month 9, and Month 12 (or Early Termination). ECG tracings or ECG machine data is collected for centralized review by a cardiologist. A 12-Lead ECG is performed (concurrent with Holter Monitor) on the day of gene delivery and on Day 2 and Day 3 post-gene delivery. Additional electrophysiological monitoring is at the discretion of the investigator as per local institutional guidelines.

### Other Clinical Assessments: 12-Lead Holter

Patients have a 12-lead continuous Holter monitor attached 24 hours prior to dose administration on Day −1. The Holter monitor remains through 48 hours (Day 3). Serial ECG data is pulled in triplicate from the Holter monitor data at the following time points: pre-dose, 2 hour, 4 hour, 6 hour, 8 hour, 12 hour, 24 hour, 36 hour, and 48 hour. Twenty-four-hour Holter monitoring is performed at screening and Months 1, 2, 3, 6, 9 and 12 visits (or Early Termination).

### Other Clinical Assessments: Echocardiogram

An echocardiogram is performed at screening/baseline, and at the Month 3, Month 6, Month 9, and Month 12 Visits (or Early Termination).

### Other Clinical Assessments: Spinal X-Ray

A spinal X-ray is performed at screening/baseline to rule out patients with severe scoliosis or those that would require major spinal surgical procedures during the 1-year study assessment period.

### Other Clinical Assessments: Pulmonary Exam

Patients are assessed by a pulmonologist at the time points specified in Table 4 and may be fitted with a non-invasive positive pressure ventilator (e.g., BiPAP) at the discretion of the pulmonologist and/or investigator. Patients requiring non-invasive ventilatory support are asked to bring the machine to each study visit such that the study staff can remove an SD card which records actual usage data. This usage data is transferred to the clinical database. Patients requiring non-invasive ventilatory support are asked to remove the SD card and ship it to the study site in instances of missed study visits.

### Fluoroscopic/Radiographic Guidance of AVXS-101 Injection

AVXS-101 intrathecal injection procedure is performed under sterile conditions under fluoroscopy by an interventional radiologist or other appropriately trained and experienced physician in accordance with institutional guidelines. Capture of radiographic images may not be required for this procedure.

### Other Clinical Assessments: Photographs of Injection Site

Photographs are taken of the injection site through Day 30 at the time points specified in Table 4 to monitor healing of the injection wound

### Other Clinical Assessments: Laboratory Assessments

Biological samples are collected throughout the trial at the time points specified in Table 4. Biological samples are collected and shipped to a central laboratory. Samples for laboratory tests on the day prior to dosing (Day −1) are collected prior to dosing and are processed locally by the site's Clinical Laboratory Improvement Amendment (CLIA)-certified local laboratory. In some cases, samples may be collected locally for immediate results or other safety or logistical concerns.

TABLE 5
Total Blood Volume

| Visit | | Tests | Total Volume | |
|---|---|---|---|---|
| Screening | | Hematology, chemistry/CK-MB or Troponin I Coagulation, | 19.3-19.6 | mL |
| | | virus serology, immunology sample (AAV9/SMN Ab only), | | |
| | | diagnostic confirmation sample | | |
| Day | 1 | Hematology, chemistry, coagulation, capillary blood gas | 6.0 | mL |
| Day | 2 | Hematology, chemistry, coagulation, capillary blood gas | 6.0 | mL |
| Day | 7 | Hematology, chemistry/CK-MB3 or Troponin I, coagulation, | 10.0-12.3 | mL |
| | | immunology sample | | |
| Day | 14 | Hematology, chemistry, coagulation immunology sample | 9.0-11.0 | mL |
| Day | 21 | Hematology, chemistry, coagulation immunology sample | 9.0-11.0 | mL |
| Day | 30 | Hematology, chemistry/CK-MB3 or Troponin I, coagulation, | 11.0-12.3 | mL |
| | | immunology sample | | |
| Month | 2 | Hematology, chemistry/CK-MB3 or Troponin I, coagulation | 6.0-6.3 | mL |
| Month | 3 | Hematology, chemistry, coagulation | 5 | mL |
| Month | 4 | Hematology, chemistry, coagulation | 5 | mL |
| Month | 5 | Hematology, chemistry, coagulation | 5 | mL |
| Month | 6 | Hematology, chemistry/CK-MB3 or Troponin I, coagulation | 6.0-6.3 | mL |
| Month | 7 | Hematology, chemistry, coagulation | 5 | mL |
| Month | 8 | Hematology, chemistry, coagulation | 5 | mL |
| Month | 9 | Hematology, chemistry/CK-MB3 or Troponin I, coagulation | 6.0-6.3 | mL |
| Month | 10 | Hematology, chemistry, coagulation | 5 | mL |
| Month | 11 | Hematology, chemistry, coagulation | 5 | mL |

| Last Study | Hematology, chemistry/CK-MB3 or Troponin I, coagulation | 6.0-6.3 | mL |
|---|---|---|---|
| Visit | | | |
| (Month 12) | | | |
| | Total Volume for Study 1-Year Duration | 135-137.1 | mL |

In a case where sufficient blood cannot be collected from a patient, blood is used in the following priority order with the first having greatest priority and last having the least priority:

1. Safety blood labs: chemistry>hematology>coagulation>CK-MB or Troponin
2. IFN-γ ELISpots to detect T-cell responses
3. Serum antibody to AAV9 and SMN
4. Genetic re-confirmation testing

If there is not sufficient blood volume to include the genetic reconfirmation testing sample at the screening visit, the patient returns before Visit 2. All patients have genetic reconfirmation testing completed.

### Other Clinical Assessments: Hematology

Hematology analysis includes a CBC with differential and platelet count with smear. Samples are collected and shipped in accord with the laboratory manual provided by the central laboratory. Immediate/same-day hematology analyses during in-patient dosing, as determined by the investigator, are performed as per investigational site standard procedures at the local laboratory.

### Other Clinical Assessments: Serum Chemistry

Samples are collected and shipped in accord with the laboratory manual provided by the central laboratory.

Immediate/same-day chemistry analyses during in-patient dosing, as determined by the investigator, are performed as per investigational site standard procedures at the local laboratory.

Chemistry analysis include the following at all study visits: Serum gamma glutamyl transferase (GGT), AST/ALT, Serum total bilirubin, Direct bilirubin, Albumin, Glucose, Total creatine kinase, Creatinine, BUN, Electrolytes, Alkaline phosphatase.

CK-MB or Troponin I is collected at screening, Day 7, Day 30, Day 60 and at Months 6, 9, and 12/End of Study. Troponin I is measured instead of CK-MB in new patients who are screened and enrolled after amendment 5 (protocol version 6.0) goes into effect. Participants who have been screened and enrolled but who have not yet received gene replacement therapy (visit #2) at the time that amendment 5 (protocol version 6.0) goes into effect have baseline troponin I testing prior to treatment with AVXS-101 and have troponin I testing in place of CK-MB. CK-MB is collected from all other participants. Investigators receive laboratory results from all study visits from the central laboratory (except Day −1).

### Other Clinical Assessments: Virus Serology

The administration of an AAV vector has the risk of causing immune-mediated hepatitis. For patients who have HIV or positive serology for hepatitis B or C or Zika virus, administration of AAV vector may represent an unreasonable risk; therefore, negative serology testing are confirmed at screening, prior to treatment. These samples are collected and shipped in accord with the laboratory manual provided by the central laboratory.

### Other Clinical Assessments: Coagulation Studies

Coagulation studies include prothrombin time (PT), partial prothrombin time (PTT), and international normalized ratio (INR) are collected in accordance with the laboratory manual provided by the central laboratory. Coagulation studies are performed as per the timepoints specified in Table 4.

### Other Clinical Assessments: Urinalysis

Urine samples are collected in accord with the laboratory manual provided by the central laboratory as per the time points specified in Table 4. Day −1 and immediate/same-day urinalyses during in-patient dosing, as determined by the investigator, are performed as per investigational site standard procedures at the local laboratory. Urinalysis includes the following parameters: Color, Clarity/turbidity, pH, Specific gravity, Glucose, Ketones, Nitrites, Leukocyte esterase, Bilirubin, Blood, Protein, Red Blood Cells, White Blood Cells, Squamous epithelial cells, Casts, Crystals, Bacteria, Yeast.

### Other Clinical Assessments: Capillary Blood Gas

Capillary blood gas is completed as per the time points specified in Table 4. A puncture or small incision is made with a lancet or similar device into the cutaneous layer of the patients' skin at a highly vascularized area (heel, finger, toe). To accelerate blood flow and reduce the difference between the arterial and venous gas pressures, the area is warmed prior to the puncture. As the blood flows freely from the puncture site, the sample is collected in a capillary tube.

### Other Clinical Assessments: ELISA: Anti-AAV9 Ab

Blood samples are collected and shipped to the central laboratory in accord with the laboratory manual to test for serum antibodies to AAV9 at screening and as per the timepoints specified in Table 4.

### Other Clinical Assessments: ELISA: Anti-SMN Ab

Blood samples are collected and shipped to the central laboratory in accord with the laboratory manual to test for serum antibodies to SMN as per the timepoints specified in Table 4.

### Other Clinical Assessments: IFN-γ ELISpots

Blood is collected and shipped to the central laboratory in accord with the laboratory manual to perform interferon gamma (IFN-γ) ELISpots to detect T-cell responses to AAV9 and SMN as per the timepoints specified in Table 4.

### Other Clinical Assessments: Baseline Screening of Mother

There is potential that the mother of the enrolled patient may have pre-existing antibodies to AAV9 that may be transferred to the patient via placental transfer in utero or theoretically through breast milk. Informed consent is requested from the mother of the patient to screen the mother for circulating antibodies to AAV9. Once informed consent has been obtained, the mother has her blood drawn from a peripheral vein and shipped to the central laboratory for screening of anti-AAV9 antibodies. If AAV9 antibodies are identified, the investigator should discuss with the mother whether to continue or to stop breastfeeding. Patients consuming banked breast milk from donor sources that cannot be tested for anti-AAV9 antibodies are transitioned to formula prior to participation.

### Other Clinical Assessments: Blood for Diagnostic Confirmation Testing

A blood sample is collected during the screening visit and shipped to the central laboratory in accord with the laboratory manual for re-confirmation of SMN1 deletions, SMN2 copy number, and absence of exon 7 gene modifier mutation (c.859G>C). This is done to ensure consistency in diagnostic testing practices.

### Other Clinical Assessments: Saliva, Urine, and Stool Collection

Studies have shown that some vector can be excreted from the body for up to a few weeks after injection; this is called “viral shedding”. Vector shedding can be found in the blood, urine, saliva, and stool for up to a week following injection. The risks associated with the shed vector are not known at this time; however, it is unlikely as the vector is non-infectious and cannot replicate. Regardless, IRB-approved instructions are provided to patient families and care givers regarding use of protective gloves if/when coming into direct contact with patient bodily fluids and/or waste as well as good hand-hygiene for a minimum of two weeks after the injection. Additionally, patients are prohibited from donating blood for two years following the vector injection.

Saliva, urine, and stool samples are collected in accord with the laboratory manual for viral shedding studies in accord with Table 4 including 24 hours and 48 post-doses. Patients at all sites 48 months who are no longer in diapers provide full volume urine and full volume feces samples at Day 7, Day 14, and Day 30 for at least one void and one defecation. Samples are prepared as per the laboratory manual, stored in a −80° C. freezer, and shipped to the central laboratory in accord with the laboratory manual. A subset of patients at sites opting to participate in the viral shedding sub-study have 24-hour total volume urine and fecal samples collected through 24 hour-post dose and 48 hours-post dose (to include all excretions in those time periods).

### Example 2—AVXS-101 Studies in SMA Patients (Clinical Trials Interim Results I)

Patients were identified, treated and evaluated as per the protocol described in Example 1. AVXS-101 was administered intrathecally to patients with spinal muscular atrophy (SMA) who could sit but not stand or walk at the time of study entry. Patients had 3 copies of the SMN2 gene in addition to biallelic deletion of SMN1. Patients were stratified in two groups, those >6 months and <24 months of age at time of dosing and those ≥24 months and <60 months of age at time of dosing. Sixteen patients >6 months and <24 months, and twelve patients ≥24<60 months were enrolled. Within the younger-age group, three patients received administration of 6.0×10¹³ vg of AVXS-101 (Dose A). The remainder of the younger patients, and all of the older patients received 1.2×10¹⁴ vg of AVXS-101 (Dose B).

Patients received AVXS-101 premixed with 1.5 mL of an appropriate contrast medium for radiographic monitoring as a one-time administration via lumbar intrathecal (IT) injection. Patients received prophylactic prednisolone for the first two months after treatment to dampen the host immune response. Safety and efficacy are evaluated periodically over a 12-month period after treatment. For patients >6 months and <24 months of age at time of dosing, an efficacy measure was the proportion of patients who achieved the ability to stand alone (Bayley Scales of Infant and Toddler Development®-Gross Motor Subset #40). Additional milestones, defined by World Health Organization Multicentre Growth Reference Study (WHO-MGRS) criterion (Wijnhoven 2004), including rolling from back to side, crawling, standing with support, pulling to stand, and walking with or without assistance, were assessed. For patients ≥24 months and <60 months of age at time of dosing, an outcome measure was the change from baseline in Hammersmith Functional Motor Scale-Expanded (HFMSE). Percent of responders (defined as achieving HFMSE score >3 points; Swoboda, et al 2010) was assessed monthly.

Patients between the ages of 6 and 24 months with SMA Type 2 were evaluated between five and 12 months after receiving Dose A (6.0×10¹³ vg; n=3) or Dose B (1.2×10¹⁴ vg; n=13) intrathecal AVXS-101. As shown in Table 6, changes in Bayley® Gross Motor Scale scores ranged between −1 and 14 points (mean increase+SD of 3.6+3.5 pts), with 14 of 16 patients (87.5%) showing improvements from baseline. Seven of 16 patients achieved at least one new Bayley® item after treatment. Two patients—one in each dose group—achieved the study endpoint of standing independently (E02, E24); one patient (E24) achieved standing before 20 months of age and now ambulates independently.

TABLE 6
Selected items of Bayley Scales of Infant and Toddler Development - Gross Motor Scale in SMA Type 2 patients aged 6 months - 24 months.

| | | | Rolls from | | Sits | | | | | | | |
|---|---|---|---|---|---|---|---|---|---|---|---|---|
| | | | Back to | Pulls up | without | Supports | | Pulls to | | Stands | | |
| | Age at | | Sides | to Sit | Support | Weight | Crawls | Stand | Walks with | Alone | Months | |
| | Injection | Sits* | (Item | (Item | (Item | (Item | (Item | (Item | Assistance | (Item | after | Change in |
| Patient | (mos) | Independently | #20) | #23) | #26) | #33) | #34) | #35) | (Item | #40) | Treatment | Bayley ® |
| E-01⁺ | 18.8 | X | X | ◯ | X | | ◯ | | | | 12 | 5 |
| E-02⁺ | 20.2 | X | X | X | X | X | ◯ | ◯ | X | ◯ | 12 | 5 |
| E-03* | 12.5 | X | X | X | X | | | | | | 12 | 7 |
| E-04 | 14.7 | X | ◯ | ◯ | X | | | | | | 11 | 11 |
| E-06 | 23.2 | X | X | | X | | | | | | 8 | 3 |
| E-09 | 20 | X | X | | X | | | | | | 7 | 3 |
| E-12 | 19.8 | X | X | X | X | | | | | | 7 | 2 |
| E-14 | 14.3 | X | ◯ | | ◯ | | | | | | 7 | 3 |
| E-15 | 12 | X | X | | X | | | | | | 7 | −1 |
| E-20 | 19.9 | X | X | | X | | | | | | 6 | 2 |
| E-21 | 20.3 | X | X | | X | | | | | | 5 | 4 |
| E-23 | 19.8 | X | X | | X | | | | | | 5 | 1 |
| E-24 | 7 | X | X | X | X | ◯ | ◯ | ◯ | ◯ | ◯ | 5 | 17 |
| E-25 | 17.1 | X | ◯ | ◯ | ◯ | ◯ | X | | | | 4 | 2 |
| E-27 | 11.9 | X | X | X | X | | | | | | 5 | 6 |
| E-28 | 15.1 | X | X | ◯ | ◯ | | | | | | 5 | 0 |

(X) denotes ability to perform the item independently prior to treatment;
(◯) represents new ability to perform the item independently after treatment.

Patients between the ages of two and five years with SMA Type 2 were evaluated between five and nine months after receiving Dose B (1.2×10¹⁴ vg; n=12) of intrathecal AVXS-101. As shown in Table 7, changes in Bayley® Gross Motor Scale scores ranged between −8 and 10 points (mean increase+SD of 2.1+1.3 pts), with nine of 12 patients (75%) showing improvement from baseline. Five of 12 patients (42%) achieved at least one new Bayley® item after treatment. Two patients (E07; E13) demonstrated ability to stand with support after treatment. One patient (E07) is now able to walk with assistance.

TABLE 7
Selected items of Bayley Scales of Infant and Toddler Development ® - Gross Motor Scale in SMA Type 2 patients aged 2 years to 5 years.

| | | | Rolls from | | Sits | | | | | | | |
|---|---|---|---|---|---|---|---|---|---|---|---|---|
| | | | back to | Pulls up | without | Supports | | Pulls to | Walks with | Stands | | |
| | Age at | | sides | to Sit | Support | Weight | Crawls | Stand | Assistance | Alone | Months | |
| | Injection | Sits* | (Item | (Item | (Item | (Item | (Item | (Item | (Item | (Item | after | Change in |
| Patient | (mos) | Independently | #20) | #23) | #26) | #33) | #34) | #35) | #37) | #40) | Treatment | Bayley ® |

| | | | | | | | | | | | |
|---|---|---|---|---|---|---|---|---|---|---|---|
| E-05 | 29.5 | X | X | | X | | | | | 9 | 1 |
| E-07 | 50 | X | X | X | X | ◯ | X | X | ◯ | 6 | 3 |
| E-08 | 35.6 | X | X | ◯ | X | | | | | 7 | 8 |
| E-10 | 45.3 | X | X | | X | | X | | | 7 | 2 |
| E-11 | 53.7 | X | X | | X | | | | | 7 | 1 |
| E-13 | 30.7 | X | X | | X | ◯ | | | | 7 | 10 |
| E-16 | 28 | X | X | ◯ | X | | X | | | 6 | 3 |
| E-17 | 32 | X | | | X | | | | | 6 | 0 |
| E-18 | 54.5 | X | X | | X | | | | | 6 | 0 |
| E-19 | 26.2 | X | X | | X | | | | | 6 | −8 |
| E-22 | 37.2 | X | X | | X | | | | | 6 | −1 |
| E-26 | 27.3 | X | ◯ | ◯ | X | | | | | 5 | 4 |

(X) denotes ability to perform the item independently prior to treatment;
(◯) represents new ability to perform the item independently after treatment.

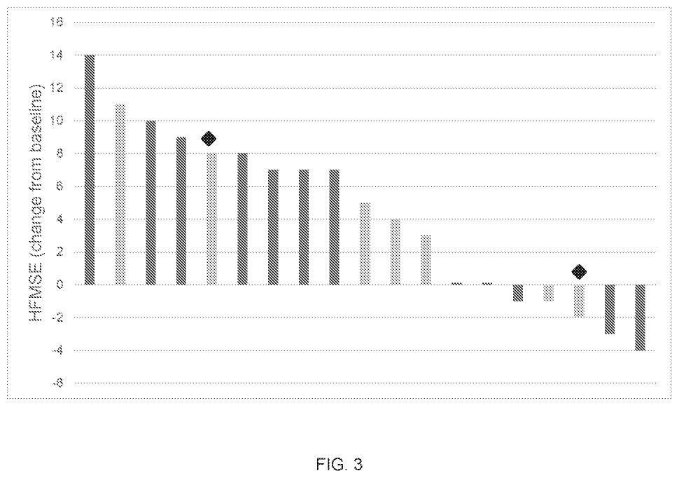

The Hammersmith Functional Motor Scale Expanded (HFMSE) was performed on patients after reaching two years of age (6 to 24 months age group) and older patients (2 to 5-year age group). Changes in HFMSE scores ranged between −4 and 14 points (mean increase+SD of 4.3+5.3 pts), with 12 of 19 patients (63.1%) showing improvement from baseline. Seven of 12 patients (58%) in the older age group (2 to 5 years) showed improvements in HFMSE, while five of seven patients (71%) in the younger (6 to 24 months) group improved. One patient (E02), treated at 20.3 months of age, achieved ability to stand unsupported. Twelve of 19 patients (63%) were considered responders (achieving an improvement on HFMSE of three points or more) (FIG. 3). A correlation between HFMSE score and age of patient at time of treatment was not found. Swoboda et al. (2010) “SMA CARNI-VAL Trial Part I: Double-Blind, Randomized, Placebo-Controlled Trial of L-Carnitine and Valproic Acid in Spinal Muscular Atrophy,” PLOS ONE 5(8): e12140.

TABLE 8
Selected Hammersmith Functional Motor Scale Expanded (HFMSE) in SMA
Type 2 patients aged 2 years to 5 years at the time of assessment.

| | | Sits | Rolls from | Sitting | Four Point | | Supported | Stands | | |
|---|---|---|---|---|---|---|---|---|---|---|
| | Age at | Independently | Side to Side | to Lying | Kneeling | Crawling | Standing | Unsupported | Months | |
| | Injection | (Item | (Items | (Item | (Item | (Item | (Item | (Item | after | Change in |
| Patient | (mos) | #1) | #6-9) | #10) | #15) | #16) | #18) | #19) | Treatment | HFMSE |
| E-01⁺ | 18.8 | XX | X | | XX | XX | ◯ | | 12 | −2 |
| E-02⁺ | 20.3 | XX | X◯ | X◯ | X◯ | ◯◯ | X◯ | ◯◯ | 12 | 8 |
| E-04 | 14.7 | XX | XX | X◯ | ◯ | | | | 11 | 5 |
| E-05 | 29.5 | XX | XX | ◯ | | | | | 9 | 7 |
| E-06 | 23.2 | XX | X◯ | ◯ | | | | | 8 | 11 |
| E-07 | 50 | XX | X◯ | X◯ | XX | XX | ◯◯ | | 6 | 7 |
| E-08 | 35.1 | XX | X◯ | ◯ | | | | | 7 | 8 |
| E-09 | 49.6 | XX | XX | ◯ | | | | | 7 | 4 |
| E-10 | 45 | XX | XX | X | XX | XX | ◯ | | 7 | 0 |
| E-11 | 53.6 | XX | X | | | | | | 7 | 0 |
| E12 | 19.8 | XX | ◯ | | | | | | 7 | 3 |
| E-13 | 30.7 | XX | X◯ | ◯◯ | ◯◯ | ◯ | ◯◯ | | 7 | 14 |
| E-16 | 28 | XX | X◯ | X◯ | XX | XX | | | 6 | 8 |
| E-17 | 31.9 | XX | | | | | | | 6 | −1 |
| E-18 | 54 | XX | X | X | X | | | | 6 | −3 |
| E-19 | 26 | XX | XX | | | | | | 6 | −4 |
| E-20 | 19.9 | XX | XX | X | | | | | 6 | −1 |
| E-22 | 37.2 | XX | ◯ | ◯ | | | | | 6 | 7 |
| E-26 | 27.2 | XX | ◯ | | | | | | 5 | 9 |

(X) denotes ability to perform the item with assistance prior to treatment;
(XX) denotes ability to perform the item independently prior to treatment.
(◯) represents new ability to perform the item with assistance after treatment;
(◯◯) represents new ability to perform the item independently after treatment.
(X◯) denotes ability to perform the item with assistance prior to treatment and new ability to perform the item without assistance after treatment.

FIG. 3 shows the HFMSE scores of individual patients as a function of patient age. Testing of HFMSE did not begin in patients in the 6 to 24 months age group until they reached 24 months of age. Sixty three percent of patients (12 of 19) showed improvements in HFMSE. One patient in Dose A (6.0×10¹³ vg) group showed improvement of eight points by eight months of treatment; a second Dose A patient declined by two points after seven months of assessment.

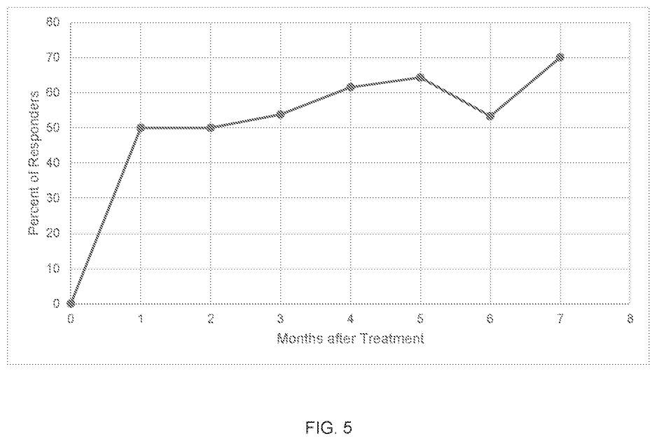

Patients who achieved at least a 3-point improvement of HFMSE were characterized as responders in this study. For the older-age cohort (two to five years of age), HFMSE was assessed from baseline through 5 months of treatment for 12 patients, and for 10 and 5 patients at months six and seven, respectively. For the younger-aged cohort (six months to two years), one patient was assessed at months three and four, and five patients were assessed at months six and seven after receiving AVXS-101 treatment. All patients in the older-aged cohort, and patients in the younger-aged cohort who reached two years of age and beyond in are shown in FIG. 5. A rapid responder rate of 50% was observed as soon as one month after treatment. The responder rate was maintained at or above 50% through seven months of study, with a trend toward increasing response rates over time.

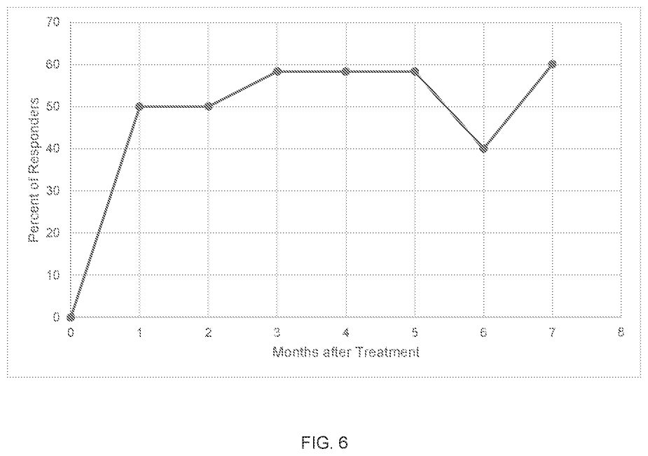

For the full cohort (n=12) from baseline through five months of treatment, the monthly responder rates for patients in the older-aged cohort (two to five years of age) for whom HFMSE assessments were performed are shown in FIG. 6. A responder rate of 50% was observed as soon as one month after treatment. With the exception of the sixth month after treatment, the responder rates were maintained at or above 50% through seven months of study. One early responder had a drop in HFMSE at the six-month evaluation, reducing the responder rate below 50% at this timepoint.

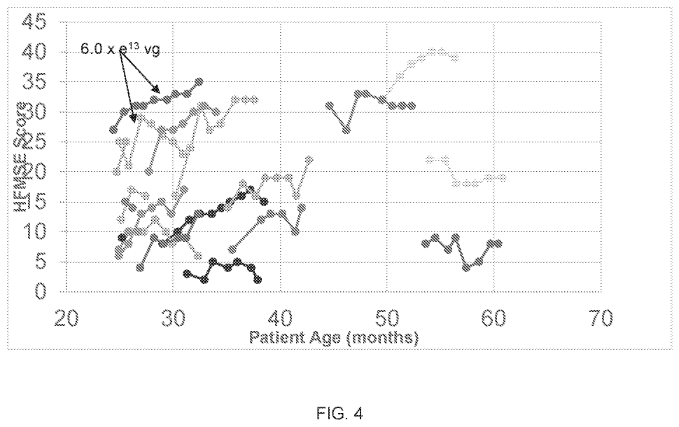

Over all, twenty-three new motor milestones were observed in 11 of 24 patients during the period of observation of four to twelve months (Tables 6 to 8). In the older-aged cohort, the mean HFMSE score increased by 4.3 points between 5 and 9 months of study (Table 8). A majority of patients in both age cohorts (63%) had improvements in HFMSE scores after treatment, irrespective of dose (FIGS. 3, 4). Fifty percent of patients in this study had clinically meaningful improvements in HFMSE (i.e. responders, with scores >3 points) after just one month of therapy, with responder rates gradually increasing over time. Treatment with AVXS-101 was more efficacious than has been reported for other therapies such as, for example, standard of care. These results demonstrate that a majority of patients had early responses to a single dose of intrathecal AVXS-101, and show a rapid onset of response, with maintenance of effect throughout the period during which intrathecally administered AVXS-101 has been studied.

### Example 3—AVXS-101 Studies in SMA Patients (Clinical Trials Interim Results II)

Further interim results of the clinical trials as detailed in Example 1 and 2 are presented here. AVXS-101 was administered intrathecally (IT) to patients with spinal muscular atrophy (SMA) who could sit unsupported for ≥10 seconds but could not stand or walk independently at the time of study entry. Patients had 3 copies of the SMN2 gene in addition to biallelic deletion of SMN1. Patients were stratified in two groups, those ≥6 months and <24 months of age at time of dosing, and those ≥24 months and <60 months of age at time of dosing. Pre-treatment baseline assessments were performed for all study patients (≥6 months and <60 months of age) using the Bayley Scales® and additional baseline assessments were performed for the ≥24 month and <60 months age group using the HFMSE.

Within these two age groups, three different therapeutic doses were administered as described: Three patients ≥6 months and <24 months of age at time of dosing received a single IT administration of 6.0×10¹³ vg of AVXS-101 (Dose A). Thirteen patients ≥6 months and <24 months of age and twelve patients ≥24 month and <60 months of age received a single IT administration of 1.2×10¹⁴ vg of AVXS-101 (Dose B). Three patients ≥6 months and <24 months of age at time of dosing received a single IT administration of 2.4×10¹⁴ vg of AVXS-101 (Dose C). In future studies, an additional 21 patients will be given Dose C, with 9 of those patients from the ≥6 months and <24 months age group at time of dosing, and 12 of those patients from the ≥24 month and <60 months age group at time of dosing.

The current study population also included 31 patients in the Intent-to-Treat (ITT) Set, which was defined as all patients who received IT AVXS-101, of whom 19 patients were ≥6 months and <24 months of age, and 12 patients were ≥24 month and <60 months of age at the time of enrollment. In addition, 4 patients (3 Dose A and 1 Dose B patient) were included in the Efficacy Completer Analysis Set (ECAS), which was defined as all patients who have completed 12 months of post-dose follow-up. All efficacy analyses were conducted using the ITT Set as the primary population and ECAS as a supportive population in the interim results.

Data from patients treated with AVXS-101 were compared with patient-level data drawn from a peer-reviewed and widely cited natural history dataset collected by the Pediatric Neuromuscular Clinical Research (PNCR) network. Kaufmann et al., “Prospective cohort study of spinal muscular atrophy types 2 and 3.” (2012) Neurology, 79(18):1889-1897. The PNCR is a large natural history study developed from a cohort of 337 patients with any form of SMA, followed at 3 large, internationally recognized tertiary medical centers with significant expertise in the management of SMA (Harvard University/Boston Children's Hospital, Columbia University and the University of Pennsylvania/Children's Hospital of Philadelphia). The data do not contain assessments using the Bayley Scales of Infant and Toddler Development®, which limits PNCR data use for the ≥6 months and <24 months age group. The SMN2 modifier mutation (c.859G>C) described by Prior and colleagues was not assessed in the PNCR study cohort. Prior et al., “A positive modifier of spinal muscular atrophy in the SMN2 gee.” (2009) A. J. Hum. Genet., 85(3):408-441.

PNCR N=51 natural history control group: For patients ≥6 months and <24 months of age, a cohort of 51 patients drawn from the PNCR natural history study was designated a “population-matched” control cohort. This comparison cohort includes all 51 patients enrolled in the PNCR study who met the criteria of: (1) having SMA types 2 or 3, (2) 3 copies of SMN2, (3) symptom onset before 12 months of age, and (4) had at least one visit at or before 36 months of age. Of this cohort, 7/51 patients (13.74%) attained the ability to stand alone, which was defined as achieving a score of 2 on item #19 of the HFMSE at any time at or before 36 months of age. The ability to walk alone was attained in 5/51 patients (10%) and was defined as achieving a HFMSE score of 2 on item #20 at any time at or before 36 months of age.

PNCR N=15 natural history control group: For patients ≥24 months and <60 months of age, patient-level data from a cohort of 15 patients drawn from the PNCR natural history study was chosen as a “population-matched” control cohort. This control group was used for the primary analyses. This natural history control group had: (1) SMA types 2 or 3, (2) 3 copies of SMN2, (3) symptom onset before 12 months of age, (4) a diagnosis of SMA before 24 months of age, and (5) inability to stand or walk at enrollment into the PNCR study. The cohort members received a HFMS or HFMSE evaluation between 24 and 60 months of age which was used as the baseline for comparison of follow-up assessments. This PNCR group of 15 patients had one patient who had an HFMSE score of 0 recorded at baseline and all follow-up visits. In 5/15 (33%) individuals from the cohort, HFMSE scores were collected for a period longer than 12 months. The final visit was 18 months for 2/15 patients (13%), 42 months for 2/15 patients (13%) and 48 months for 1/15 patients (7%).

PNCR N=17 natural history control group: For patients ≥24 months and <60 months of age, patient-level data from a cohort of 17 patients drawn from the PNCR study were identified in order to improve matching between the patient group and the natural history controls. This control group was used for sensitivity analyses. Twelve patients originally in the PNCR N=15 control group were in the PNCR N=17 natural history control group. Three patients originally in the PNCR N=15 control group were not included (the one individual with HFMSE=0 for baseline and follow up visits, 2 individuals with final visits >12 months). These 17 individuals had age, clinical, and genetic criteria that were matched as closely as possible to the study group. The first visit within the ≥24 months and <60 months age range was defined as the baseline visit. Subsequent visits within a 12-month interval were used to determine change from baseline for HFMSE. Clinically, these individuals were able to sit but could not stand or walk independently. Genetically, patients harbored biallelic SMN1 deletions and 3 copies of SMN2. A limitation of using PNCR natural history controls was that evaluation intervals were not consistent among participants. Hence, some individuals in this control group had months of data (See e.g., Table 13).

The patient disposition by treatment and by age for all enrolled patients is detailed in Table 9. A summary of demographic and baseline characteristics by treatment by age group for the Safety Analysis Set is provided in Table 10.

TABLE 9
Patient Disposition - All patients (Interim Results II cutoff)

| | | Dose B | | | Dose C | | | |
|---|---|---|---|---|---|---|---|---|
| | Dose A | | | Age ≥24 | | Age ≥24 | | |
| | Age <24 | Age <24 | | and <60 | Age <24 | and <60 | | |
| | months | months | | months | months | months | Overall | |
| Patients Screened | | | | | | | 36 | |
| Patient Screen Failures | | | | | | | 5 | |
| Patients in the | 3 | 13 | | 12 | 3 | 0 | 31 | |
| Enrolled Set (n (%)) | | | | | | | | |
| Patients in the ITT Set | 3 (100) | 13 | (100) | 12 (100) | 3 (100) | 0 | 31 | (100) |
| (n (%)) | | | | | | | | |
| Patients in the Full | 3 (100) | 13 | (100) | 12 (100) | 3 (100) | 0 | 31 | (100) |
| Analysis Set (n (%)) | | | | | | | | |
| Patients in the Safety | 3 (100) | 13 | (100) | 12 (100) | 3 (100) | 0 | 31 | (100) |
| Analysis Set (n (%)) | | | | | | | | |
| Patients in the | 3 (100) | 1 | (7.7) | 0 | 0 | 0 | 4 | (12.9) |
| Efficacy Completer | | | | | | | | |
| Analysis Set (n (%)) | | | | | | | | |
| Patients completed | 3 (100) | 1 | (7.7) | 0 | 0 | 0 | 4 | (12.9) |
| the study thus far (n | | | | | | | | |
| (%)) | | | | | | | | |

| | | | | | | |
|---|---|---|---|---|---|---|
| Patients discontinued | 0 | 0 | 0 | 0 | 0 | 0 |
| from the study (n (%)) | | | | | | |

TABLE 10
Demographics and Baseline Characteristics - Safety Analysis Set

| Demographic/ | Dose A | | Dose B |
|---|---|---|---|
| Characterics | | | Age ≥24 |
| Category/ | Age <24 | Age <24 | and <60 |
| Statistic | months | months | months |

Age (months)

| n | 3 | | 13 | | 12 | |
|---|---|---|---|---|---|---|
| Mean (SD) | 15.67 | (4.041) | 15.46 | (4.427) | 35.92 | (10.483) |
| Median (Min, Max) | 18.00 | (11.0, 18.00) | 16.00 | (6.0, 22.0) | 32.00 | (25.0, 53.0) |

Gender (n (%))

| | | | | | | |
|---|---|---|---|---|---|---|
| Male | 1 | (33.3) | 7 | (53.8) | 6 | (50.0) |
| Female | 2 | (66.7) | 6 | (46.2) | 6 | (50.0) |

Ethnicity (n (%))

| Hispanic or Latino | 2 | (66.7) | 3 | (23.1) | 0 | |
|---|---|---|---|---|---|---|
| No Hispanic or | 1 | (33.3) | 10 | (76.9) | 12 | (100) |
| Latino | | | | | | |

Race (n (%))

| | | | | | | |
|---|---|---|---|---|---|---|
| White | 2 | (66.7) | 10 | (76.9) | 8 | (66.7) |

| | | | | | |
|---|---|---|---|---|---|
| Asian | 0 | 1 | (7.7) | 4 | (33.3) |

| Other | 0 | | 1 | (7.7) | 0 |
|---|---|---|---|---|---|
| Multiple | 1 | (33.3) | 1 | (7.7) | 0 |

Baseline weight (kg)

| n | 3 | | 13 | | 12 | |
|---|---|---|---|---|---|---|
| Mean (SD) | 9.90 | (1.900) | 9.67 | (0.778) | 13.36 | (3.235) |
| Median (Min, Max) | 9.90 | (8.0, 11.8) | 9.50 | (8.3, 10.8) | 12.70 | (9.8, 20.2) |

Baseline length/height (cm)

| n | 3 | | 13 | | 12 | |
|---|---|---|---|---|---|---|
| Mean (SD) | 76.63 | (4.744) | 77.12 | (5.308) | 92.28 | (8.449) |
| Median (Min, Max) | 74.90 | (73.0, 82.0) | 75.50 | (69.0, 87.0) | 89.00 | (82.5, 112.0) |

Baseline BMI (kg/m²)

| n | 3 | | 13 | | 12 | |
|---|---|---|---|---|---|---|
| Mean (SD) | 16.736 | (1.4937) | 16.363 | (1.6485) | 15.530 | (1.9429) |
| Median (Min, Max) | 17.549 | (15.01, 17.65) | 16.576 | (12.55, 18.90) | 15.223 | (12.78, 18.66) |

Familial History of SMA including affected siblings or parent carriers (n [%])

| | | | | | | |
|---|---|---|---|---|---|---|
| Yes (n (%)) | 1 | (33.3) | 1 | (7.7) | 1 | (8.3) |
| No (n (%)) | 1 | (33.3) | 12 | (92.3) | 11 | (91.7) |

| | | | | |
|---|---|---|---|---|
| Unknown (n (%)) | 1 | (33.3) | 0 | 0 |

Gestational age at birth (weeks)

| n | 3 | | 13 | | 11 | |
|---|---|---|---|---|---|---|
| Mean (SD) | 38.33 | (1.155) | 39.15 | (0.899) | 39.45 | (2.162) |
| Median (Min, Max) | 39.00 | 37.0, 39.0) | 39.00 | (38.0, 41.0) | 40.00 | (35.0, 42.0) |

Birth Weight (kg)

| n | 3 | | 12 | | 11 | |
|---|---|---|---|---|---|---|
| Mean (SD) | 3.193 | (0.3722) | 3.699 | (0.8065) | 3.248 | (0.5360) |
| Median (Min, Max) | 3.240 | (2.80, 3.54) | 3.590 | (3.10, 6.13) | 3.200 | (2.55, 4.20) |

Birth Length (cm)

| n | 3 | | 9 | | 7 | |
|---|---|---|---|---|---|---|
| Mean (SD) | 50.557 | (2.2748) | 50.459 | (1.9058) | 51.261 | (2.2702) |
| Median (Min, Max) | 50.170 | (48.50, 53.00) | 51.000 | (47.00, 52.07) | 51.000 | (48.26, 55.50) |

Head Circumference at birth (cm)

| n | 3 | | 5 | | 7 | |
|---|---|---|---|---|---|---|
| Mean (SD) | 36.880 | (3.4063) | 34.464 | (0.8328) | 34.814 | (1.5356) |
| Median (Min, Max) | 36.000 | (34.00, 40.64) | 34.800 | (33.02, 35.00) | 34.000 | (33.00, 36.70) |

Patient reported hospitalizations (n [%])

| | | | | | | |
|---|---|---|---|---|---|---|
| Yes (n (%)) | 1 | (33.3) | 4 | (30.8) | 5 | (41.7) |
| No (n (%)) | 2 | (66.7) | 9 | (69.2) | 7 | (58.3) |

Patient reported feeding support (n [%])

| Yes (n (%)) | 0 | | 0 | | 0 | |
|---|---|---|---|---|---|---|
| No (n (%)) | 3 | (100) | 13 | (100) | 12 | (100) |

Patient reported ventilatory support (n [%])

| Yes (n (%)) | 0 | | 0 | | 1 | (8.3) |
|---|---|---|---|---|---|---|
| No (n (%)) | 3 | (100) | 13 | (100) | 11 | (91.7) |

| Demographic/ | | | | | |
|---|---|---|---|---|---|
| Characterics | Dose B | | Dose C | | |
| Category/ | Age <24 | | Age <24 | | |
| Statistic | months | | months | Overall | |
| Age (months) | | | | | |
| n | 3 | | — | 31 | |
| Mean (SD) | 18.00 | (3.464) | — | 23.65 | (12.200) |
| Median (Min, Max) | 16.0 | (16.0, 22.0) | — | 19.00 | (6.0, 53.0) |
| Gender (n (%)) | | | | | |
| Male | 3 | (100) | | 17 | (54.8) |
| Female | 0 | | | 14 | (45.2) |
| Ethnicity (n (%)) | | | | | |
| Hispanic or Latino | 0 | | 0 | 5 | (16.1) |
| No Hispanic or | 3 | (100) | 0 | 26 | (83.9) |
| Latino | | | | | |
| Race (n (%)) | | | | | |
| White | 2 | (66.7) | 0 | 22 | (71.0) |
| Asian | 1 | (33.3) | 0 | 6 | (19.4) |
| Other | 0 | | 0 | 1 | (3.2) |
| Multiple | 0 | | 0 | 2 | (6.5) |
| Baseline weight (kg) | | | | | |
| n | 3 | | — | 31 | |
| Mean (SD) | 9.23 | (0.252) | — | 11.08 | (2.783) |
| Median (Min, Max) | 9.20 | (9.0, 9.5) | — | 10.10 | (8.0, 20.2) |
| Baseline length/height (cm) | | | | | |
| n | 3 | | — | 31 | |
| Mean (SD) | 74.50 | (2.500) | — | 82.68 | (9.998) |
| Median (Min, Max) | 74.50 | (72.0, 77.0) | — | 81.00 | (69.0, 112.0) |
| Baseline BMI (kg/m²) | | | | | |
| n | 3 | | — | 31 | |
| Mean (SD) | 16.653 | (0.6724) | — | 16.105 | (1.6973) |
| Median (Min, Max) | 16.576 | (16.02, 17.36) | — | 16.139 | (12.55, 18.90) |
| Familial History of SMA including affected siblings or parent carriers (n [%]) | | | | | |
| Yes (n (%)) | 0 | | 0 | 3 | (9.7) |
| No (n (%)) | 2 | (66.7) | 0 | 26 | (83.9) |
| Unknown (n (%)) | 1 | (33.3) | 0 | 2 | (6.5) |
| Gestational age at birth (weeks) | | | | | |
| n | 3 | | — | 30 | |
| Mean (SD) | 40.00 | (1.000) | — | 39.27 | (1.507) |
| Median (Min, Max) | 40.00 | (39.0, 41.0) | — | 39.00 | (35.0, 42.0) |
| Birth Weight (kg) | | | | | |
| n | 3 | | — | 29 | |
| Mean (SD) | 3.483 | (0.2937) | — | 3.453 | (0.6507) |
| Median (Min, Max) | 3.430 | (3.22, 3.80) | — | 3.410 | (2.55, 6.13) |
| Birth Length (cm) | | | | | |
| n | 3 | | — | 22 | |
| Mean (SD) | 49.520 | (2.1478) | — | 50.600 | (2.0272) |
| Median (Min, Max) | 48.300 | (48.26, 52.00) | — | 51.000 | (47.00, 55.50) |
| Head Circumference at birth (cm) | | | | | |
| n | 2 | | — | 17 | |
| Mean (SD) | 34.750 | (1.0607) | — | 35.068 | (1.8300) |
| Median (Min, Max) | 34.750 | (34.00, 35.50) | — | 34.800 | (33.00, 40.64) |
| Patient reported hospitalizations (n [%]) | | | | | |
| Yes (n (%)) | 1 | (33.3) | 0 | 11 | (35.5) |
| No (n (%)) | 2 | (66.7) | 0 | 20 | (64.5) |
| Patient reported feeding support (n [%]) | | | | | |
| Yes (n (%)) | 0 | | 0 | 0 | |
| No (n (%)) | 3 | (100) | 0 | 31 | (100) |
| Patient reported ventilatory support (n [%]) | | | | | |
| Yes (n (%)) | 0 | | 0 | 1 | (3.2) |
| No (n (%)) | 3 | (100) | 0 | 30 | (96.8) |

Interim Results: ≥6 months and <24 months group interim assessment of primary efficacy endpoint (Doses A, B, and C; total n = 19)

The primary efficacy endpoint for this age group was attainment of Bayley Scales of Infant and Toddler Development®—Gross Motor Subset Item #40, “stand without support for at least 3 seconds.” Patients were considered to have achieved this milestone if the milestone was attained at any time during the 12-month post-dose follow-up. Video recordings of the study site assessment of milestones were confirmed by an independent central reviewer.

### Primary Efficacy Results by Dose for the ITT Set are Summarized Below and in Table 11:

For Dose A (6.0×10¹³ vg of AVXS-101), 1 of 3 patients (33.3%), patient 007-001, achieved standing with support at 11 months post-treatment. This patient was approximately 20 months of age when dosed. Although the patient did not stand alone, this patient achieved the following skills at study entry: supporting weight (Bayley® #33), walking with support (Bayley® #37), and walking sideways with support (Bayley® #38).

For Dose B (1.2×10¹⁴ vg of AVXS-101), 1 of 13 patients (7.7%), patient 007-002, achieved standing without support within 3 months post-treatment. This patient was approximately 7 months of age when dosed. According to the study physician, this patient had no manifestations of SMA identified with the neurological examination. Since the patient had an affected sibling, the patient was diagnosed early in life with genetic testing and followed with nerve conduction studies. Prior to study entry, the patient's compound muscle action potential (CMAP) was abnormal.

For Dose C (2.4×10¹⁴ vg of AVXS-101), no patients (0 of 3) achieved the milestone of standing without support at assessments up to 12 months post-treatment (Table 11).

For Dose B+Dose C, 1 of 16 patients (6.3%), patient 007-002 (described above), achieved the milestone of standing without support at 3 months post-treatment.

TABLE 11
Proportion of patients <24 months of age at time of dosing achieving the
ability to stand alone at any post-baseline visit up to 12 months - ITT set

| | | PNCR Natural | | | | | | | | |
|---|---|---|---|---|---|---|---|---|---|---|
| | | History Controls | | Dose A | Dose B | | Dose C | | Dose B + C | |
| Assessment | Statistics | (n = 51) | | (n = 3) | (n = 13) | | (n = 3) | | (n = 16) | |
| Proportion of | Yes | 7 | (13.7) | 1 (33.3) | 1 | (7.7) | 0 | | 1 | (6.3) |
| patients | No | 44 | (86.3) | 2 (66.7) | 12 | (92.3) | 3 | (100.0) | 15 | (93.8) |
| achieving | | | | | | | | | | |
| the ability to | | | | | | | | | | |
| stand alone | | | | | | | | | | |

| | | | | | | | |
|---|---|---|---|---|---|---|---|
| Proportion | Difference in proportions (95% CI) | −6.0 | (−21.8, 22.8) | −13.7 | (−28.9, 56.5) | −7.5 | (−22.0, 17.2) |

| | | | | |
|---|---|---|---|---|
| difference | p-value (Fisher's exact test) | >0.9999 | >0.9999 | 0.6687 |
| test * | | | | |

* The Fisher's exact test was performed only for Doses B, C, and B + C.

For natural history controls with SMA types 2 and 3 from the PNCR N=51 data set, 7 of the 51 patients (13.7%) achieved the milestone of standing without support (as shown in Table 11).

Statistical analysis was performed according to the protocol using a Fisher's exact test for the comparison between groups of the proportion of patients achieving the milestone (primary efficacy endpoint) and a Kaplan-Meier analysis for the supportive efficacy endpoint. The primary efficacy endpoint of achieving the ability to stand independently at any post-baseline visit up 12 months is summarized in Table 11.

The time to achieving the ability to stand alone was summarized for all patients in the PNCR group as well as by dose in the ITT Set. Using a Cox proportional hazards model to assess the treatment difference with patient age at baseline as a covariate, the hazard ratio (95% CI) was 0.43 (0.05, 3.93) for Dose B, 0 (0, not evaluable) for Dose C, and 0.37 (0.04, 3.39) for Dose B+Dose C groups, with p-values of 0.4576, 0.9951, and 0.3826, respectively. Most of the study patients had not achieved the milestone of standing independently as of the reporting of the interim results, prohibiting calculation of values such as the 25th percentile, median, and 75th percentile.

Interim Results: ≥24 Months and <60 Months Group Interim Assessment of Primary Efficacy Endpoint (Dose B; Total n=12)
a. Primary Efficacy Analysis with PNCR N=15 Natural History Control Group.

The primary efficacy endpoint for this age group was the change from baseline in HFMSE at Month 12. The baseline, post-baseline, and change from baseline values in HFMSE are summarized and analyzed using the ITT Set. The PNCR N=15 natural history control group is used as the primary “population matched” control cohort for the analyses specified in the protocol.

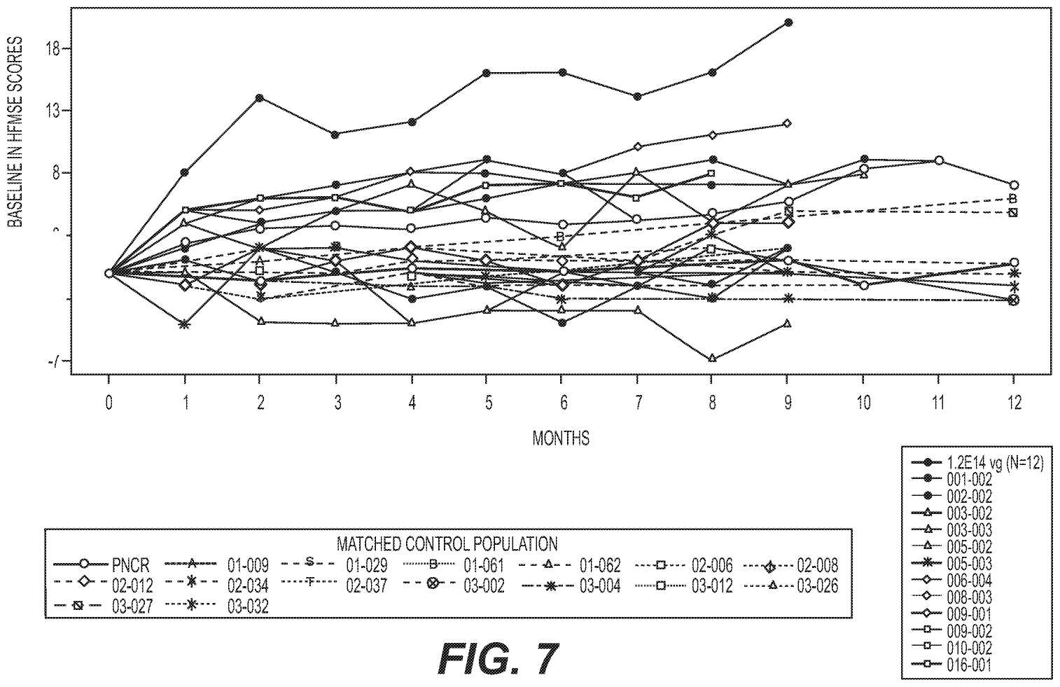

A spaghetti plot of the change from baseline in HFMSE scores up to Month 12 for individuals treated with AVXS-101 Dose B and the PNCR N=15 natural history controls is displayed in FIG. 7. Descriptive statistics for the treated patients and controls are provided in Table 12.

In the PNCR N=15 natural history controls, the mean±standard deviation (SD) for the baseline HFMSE score was 11.8±7.34. In this PNCR control group, the change from baseline HFMSE score could be calculated at Month 2 (−0.6±1.35), Month 4 (0.4±0.98), Month 6 (0.2±1.72), Month 9 (1.0±2.16), and Month 12 (0.8±2.86).

In the AVXS-101 Dose B treatment group, the baseline HFMSE value was 14.8±9.98. Most treated patients had up to 8 months of HFMSE data (11/12). The HFMSE score change from baseline at Months 2, 4, 6, 9 and 12 were 3.5±4.38, 3.6±5.07, 3.9±5.85, 5.7±6.72, and 7, respectively. The Dose B treatment group showed a robust increase in HFMSE scores as compared to the PNCR N=15 natural history control group.

TABLE 12
HFMSE values at specified time points (patients ≥24
months and <60 months of age) - ITT set - Dose B

| | PNCR Natural | | | | | |
|---|---|---|---|---|---|---|
| | History Controls | | | Dose B | | |
| | (N = 15) | | | (N = 12) | | |
| Visit | | Mean | Median | | Mean | Median |
| Assessment | n | (SD) | (Min, Max) | n | (SD) | (Min, Max) |

Baseline

| | | | | | | | | | | |
|---|---|---|---|---|---|---|---|---|---|---|
| Observed scores | 15 | 11.8 | (7.34) | 9.0 | (0, 22) | 12 | 14.8 | (9.98) | 12.0 | (3, 32) |

Month 1

| | | | | | | | | |
|---|---|---|---|---|---|---|---|---|
| Observed scores | NA | NA | NA | 12 | 17.2 | (10.05) | 15.0 | (2, 36) |
| Change from | NA | NA | NA | 12 | 2.4 | (3.34) | 3.0 | (−4, 8) |
| baseline scores | | | | | | | | |

Month 2

| | | | | | | | | | | |
|---|---|---|---|---|---|---|---|---|---|---|
| Observed scores | 10 | −13.9 | (6.30) | 15.5 | (5, 21) | 12 | 18.3 | (11.04) | 14.5 | (5, 38) |
| Change from | 10 | −0.6 | (1.35) | −1.0 | (−2, 2) | 12 | 3.5 | (4.38) | 3.0 | (−4, 14) |
| baseline scores | | | | | | | | | | |

Month 3

| | | | | | | | | |
|---|---|---|---|---|---|---|---|---|
| Observed scores | NA | NA | NA | 12 | 18.5 | (10.94) | 15.5 | (4, 39) |
| Change from | NA | NA | NA | 12 | 3.8 | (3.93) | 5.0 | (−4, 11) |
| baseline scores | | | | | | | | |

Month 4

| | | | | | | | | | | |
|---|---|---|---|---|---|---|---|---|---|---|
| Observed scores | 7 | 14.1 | (7.15) | 15.0 | (4, 23) | 12 | 18.3 | (11.83) | 15.5 | (4, 40) |
| Change from | 7 | 0.4 | (0.98) | 0.0 | (−1, 2) | 12 | 3.6 | (5.07) | 5.0 | (−4, 12) |
| baseline scores | | | | | | | | | | |

Month 5

| | | | | | | | | |
|---|---|---|---|---|---|---|---|---|
| Observed scores | NA | NA | NA | 12 | 19.3 | (11.69) | 16.5 | (4, 40) |
| Change from | NA | NA | NA | 12 | 4.5 | (5.79) | 5.5 | (−3, 16) |
| baseline scores | | | | | | | | |

Month 6

| | | | | | | | | | | |
|---|---|---|---|---|---|---|---|---|---|---|
| Observed scores | 6 | 10.5 | (7.69) | 9.5 | (0, 22) | 12 | 18.7 | (11.72) | 15.5 | (2, 39) |
| Change from | 6 | 0.2 | (1.72) | 0.0 | (−2, 3) | 12 | 3.9 | (5.85) | 4.5 | (−4, 16) |
| baseline scores | | | | | | | | | | |

Month 7

| | | | | | | | | | |
|---|---|---|---|---|---|---|---|---|---|
| Observed scores | 1 | 21.0 | 21 | (21, 21) | 11 | 17.5 | (10.14) | 16.0 | (4, 32) |
| Change from | 1 | −1.0 | −1.0 | (−1, −1) | 11 | 4.3 | (5.35) | 4.0 | (−3.14) |
| baseline scores | | | | | | | | | |

Month 8

| | | | | | | | | | |
|---|---|---|---|---|---|---|---|---|---|
| Observed scores | 1 | 20 | 20 | (20, 20) | 11 | 20.5 | (11.89) | 17.0 | (7, 39) |
| Change from | 1 | 2.0 | 2.0 | (2, 2) | 11 | 4.7 | (6.48) | 4.0 | (−7, 16) |
| baseline scores | | | | | | | | | |

Month 9

| | | | | | | | | | | |
|---|---|---|---|---|---|---|---|---|---|---|
| Observed scores | 7 | 13.7 | (7.78) | 16.0 | (2, 22) | 10 | 22.3 | (11.76) | 19.5 | (7, 39) |
| Change from | 7 | 1.0 | (2.16) | 1.0 | (−2, 5) | 10 | 5.7 | (6.72) | 5.5 | (−4, 20) |
| baseline scores | | | | | | | | | | |

Month 10

| | | | | | | | | | |
|---|---|---|---|---|---|---|---|---|---|
| Observed scores | 1 | 21.0 | 21 | (21, 21) | 3 | 26.3 | (12.10) | 22.0 | (17, 40) |
| Change from | 1 | −1.0 | −1.0 | (−1, −1) | 3 | 8.3 | (0.58) | 8.0 | (8, 9) |
| baseline scores | | | | | | | | | |

Month 11

| | | | | | | | |
|---|---|---|---|---|---|---|---|
| Observed scores | NA | NA | NA | 1 | 17.0 | 17.0 | (17, 17) |
| Change from | NA | NA | NA | 1 | 9.0 | 9.0 | (9, 9) |
| baseline scores | | | | | | | |

Month 12

| | | | | | | | | | |
|---|---|---|---|---|---|---|---|---|---|
| Observed scores | 9 | 10.2 | (7.36) | 10.0 | (0, 22) | 1 | 15.0 | 15.0 | (15, 15) |
| Change from | 9 | 0.8 | (2.86) | 0.0 | (−2, 6) | 1 | 7.0 | 7.0 | (7, 7) |
| baseline scores | | | | | | | | | |

b. Sensitivity Analysis Using the PNCR N=17 Natural History Control Group

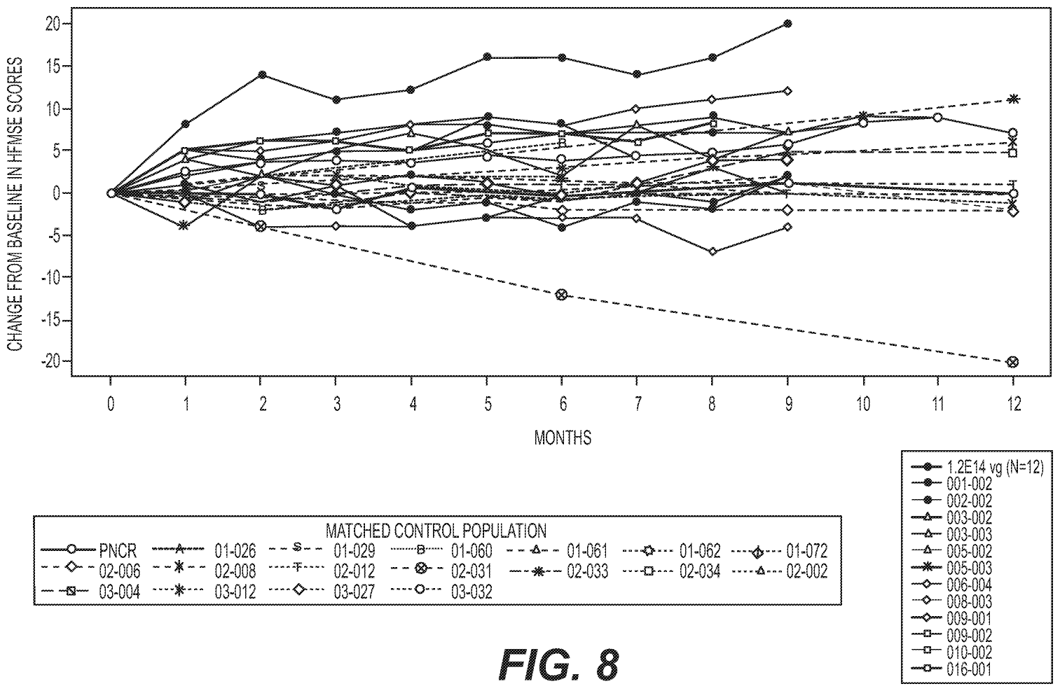

Descriptive statistics and spaghetti plots for Dose B and the PNCR N=17 natural history controls are given in Table 13 and FIG. 8.

In the PNCR N=17 natural history control group, the baseline HFMSE score was 12.1±9.21. The mean changes from baseline HFMSE score could be calculated at Month 2 (−0.2±1.56), Month 4 (0.5±1.05), Month 6 (−0.4±5.32), Month 9 (1.1±2.03), and Month 12 (−0.2±8.11). Forty one percent (7/17) of PNCR patients did not have a 12-month HFMSE score.

The AVXS-101 Dose B treatment group had a HFMSE baseline score of 14.8±9.98. The mean HFMSE score change from baseline at Months 2, 4, 6, 9, and 12 was 3.5±4.38, 3.6±5.07, 3.9±5.85, 5.7±6.72, and 7, respectively.

The Dose B treatment group showed a robust increase in HFMSE scores as compared to the PNCR N=17 natural history control group.

TABLE 13
HFMSE values at specified time points (patients ≥24 months and
<60 months of age) - ITT set (Sensitivity PNCR) - Dose B

| | PNCR Natural | | | | | |
|---|---|---|---|---|---|---|
| | History Controls | | | Dose B | | |
| | (N = 17) | | | (N = 12) | | |
| | | Mean | Median | | Mean | Median |
| Visit Assessment | n | (SD) | (Min, Max) | n | (SD) | (Min, Max) |

Baseline

| | | | | | | | | | | |
|---|---|---|---|---|---|---|---|---|---|---|
| Observed scores | 17 | 12.1 | (9.21) | 8.0 | (2, 39) | 12 | 14.8 | (9.98) | 12.0 | (3, 32) |

Month 1

| | | | | | | | | |
|---|---|---|---|---|---|---|---|---|
| Observed scores | NA | NA | NA | 12 | 17.2 | (10.05) | 15.0 | (2, 36) |
| Change from | NA | NA | NA | 12 | 2.4 | (3.34) | 3.0 | (−4, 8) |
| baseline scores | | | | | | | | |

Month 2

| | | | | | | | | | | |
|---|---|---|---|---|---|---|---|---|---|---|
| Observed scores | 9 | 12.1 | (6.21) | 8.0 | (5, 21) | 12 | 18.3 | (11.04) | 14.5 | (5, 38) |
| Change from | 9 | −0.2 | (1.56) | −1.0 | (−2, 2) | 12 | 3.5 | (4.38) | 3.0 | (−4, 14) |
| baseline scores | | | | | | | | | | |

Month 3

| | | | | | | | | | |
|---|---|---|---|---|---|---|---|---|---|
| Observed scores | 1 | 2.0 | 2.0 | (2, 2) | 12 | 18.5 | (10.94) | 15.5 | (4, 39) |
| Change from | 1 | −2.0 | −2.0 | (−2, −2) | 12 | 3.8 | (3.93) | 5.0 | (−4, 11) |
| baseline scores | | | | | | | | | |

Month 4

| | | | | | | | | | | |
|---|---|---|---|---|---|---|---|---|---|---|
| Observed scores | 6 | 12.8 | (6.85) | 12.5 | (4, 23) | 12 | 18.3 | (11.83) | 15.5 | (4, 40) |
| Change from | 6 | 0.5 | (1.05) | 0.5 | (−1, 2) | 12 | 3.6 | (5.07) | 5.0 | (−4, 12) |
| baseline scores | | | | | | | | | | |

Month 5

| | | | | | | | | |
|---|---|---|---|---|---|---|---|---|
| Observed scores | NA | NA | NA | 12 | 19.3 | (11.69) | 16.5 | (4, 40) |
| Change from | NA | NA | NA | 12 | 4.5 | (5.79) | 5.5 | (−3, 16) |
| baseline scores | | | | | | | | |

Month 6

| | | | | | | | | | | |
|---|---|---|---|---|---|---|---|---|---|---|
| Observed scores | 8 | 13.6 | (7.42) | 10.5 | (6, 27) | 12 | 18.7 | (11.72) | 15.5 | (2, 39) |
| Change from | 8 | −0.4 | (5.32) | 0.5 | (−12, 6) | 12 | 3.9 | (5.85) | 4.5 | (−4, 16) |
| baseline scores | | | | | | | | | | |

Month 7

| | | | | | | | | |
|---|---|---|---|---|---|---|---|---|
| Observed scores | NA | NA | NA | 11 | 17.5 | (10.14) | 16.0 | (4, 32) |
| Change from | NA | NA | NA | 11 | 4.3 | (5.35) | 4.0 | (−3, 14) |
| baseline scores | | | | | | | | |

Month 8

| | | | | | | | | |
|---|---|---|---|---|---|---|---|---|
| Observed scores | NA | NA | NA | 11 | 20.5 | (11.89) | 17.0 | (7, 39) |
| Change from | NA | NA | NA | 11 | 4.7 | (6.48) | 4.0 | (−7, 16) |
| baseline scores | | | | | | | | |

Month 9

| | | | | | | | | | | |
|---|---|---|---|---|---|---|---|---|---|---|
| Observed scores | 8 | 12.8 | (7.70) | 13.0 | (2, 22) | 10 | 22.3 | (11.76) | 19.5 | (7, 39) |
| Change from | 8 | 1.1 | (2.03) | 1.0 | (−2, 5) | 10 | 5.7 | (6.72) | 5.5 | (−4, 20) |
| baseline scores | | | | | | | | | | |

Month 10

| | | | | | | | | |
|---|---|---|---|---|---|---|---|---|
| Observed scores | NA | NA | NA | 3 | 26.3 | (12.10) | 22.0 | (17, 40) |
| Change from | NA | NA | NA | 3 | 8.3 | (0.58) | 8.0 | (8, 9) |
| baseline scores | | | | | | | | |

Month 11

| | | | | | | | |
|---|---|---|---|---|---|---|---|
| Observed scores | NA | NA | NA | 1 | 17.0 | 17.3 | (17, 7) |
| Change from | NA | NA | NA | 1 | 9.0 | 9.0 | (9, 9) |
| baseline scores | | | | | | | |

Month 12

| | | | | | | | | | |
|---|---|---|---|---|---|---|---|---|---|
| Observed scores | 10 | 13.6 | (7.53) | 14.0 | (1, 25) | 1 | 15.0 | 15.0 | (15, 15) |
| Change from | 10 | −0.2 | (8.11) | 0.0 | (−20, 11) | 1 | 7.0 | 7.0 | (7, 7) |
| baseline scores | | | | | | | | | |

### Interim Results: Secondary Efficacy Endpoint—Motor Milestone, Walking Independently for at Least 5 Steps

The secondary efficacy endpoint was a Bayley Scales of Infant and Toddler Development®—Gross Motor Subset Item #43 (“walks independently ≥5 steps”) for both the ≥6 months and <24 months age group and the ≥24 and <60 months age group. This milestone was scored at any post-treatment visit up to the 12-month study visit. Video evidence of the initial milestone assessment was reviewed and confirmed by an independent central reviewer.

For patients aged ≥6 months and <24 months at time of dosing, a single patient (007-002) who received Dose B (1.2×10¹⁴ vg) walked without assistance by the Month 4 visit (See patient description in previous section). The proportion of patients achieving the ability to walk without assistance was 0% (0/3) for Dose A (6.0×10¹³ vg), 7.7% (1/13) for Dose B (1.2×10¹⁴ vg) and 0% (0/3) for Dose C (2.4×10¹⁴ vg). The PNCR N=51 natural history control group was used for this analysis. Five of 51 (9.8%) patients of this control group walked independently at baseline. During the follow up period, no patient in this control group walked independently.

For patients aged ≥24 months and <60 months at time of dosing, all patients received Dose B (1.2×10¹⁴ vg). No patients in this age group received Dose C. None of the patients treated with Dose B walked independently. No patients in the primary PNCR N=15 natural history control group or in the sensitivity PNCR N=17 natural history control group walked independently.

### Interim Results: Exploratory Efficacy Endpoint—Bayley Scales of Infant and Toddler Development® Assessment

For the ≥6 months and <24 months age group and the ≥24 and <60 months age group, the change from baseline in fine and gross motor components of the Bayley Scales of Infant and Toddler Development®, Third Edition (Bayley®-III) were assessed. For the ≥6 months and <24 months age group, the second exploratory endpoint is the change in HFMSE from baseline among those patients who continue in the study past 24 months of age and had at least 6 months' worth of post-baseline HFMSE assessments recorded. Since the Bayley Scales® were not assessed in the PNCR dataset, only descriptive statistics are provided for patients <24 months of age.

Although SMA type 1 patients have severe fine motor impairment with infants being unable to grasp using their whole hand, fine motor function is relatively well preserved in SMA type 2 and SMA type 3 as reflected in the Bayley® scores for fine motor development. De Sanctis et al., “Developmental milestones in type I spinal muscular atrophy.” (2016) Neuromuscul. Disord. 26(11):754-759; Chabanon et al., “Prospective and longitudinal natural history study of patients with Type 2 and 3 spinal muscular atrophy: Baseline data NatHis-SMA study.” (2018) PLoS ONE, 13(7): e0201004. In SMA type 2 and type 3, proximal muscle dysfunction is significantly greater than distal muscle dysfunction as reflected in the Bayley® scores for gross motor development.

a. Patients Aged ≥6 Months and <24 Months at Time of Dosing

Dose A (6.0×10¹³ vg): All 3 patients in this group completed the post-dosing 12-month evaluation period. The change from baseline in Bayley Scales® at Month 12 was 12.3±6.51 for the fine motor subtest and 5.7±1.15 for the gross motor subtest.

Dose B (1.2×10¹⁴ vg): The change from baseline in the fine motor subtest was available for all 13 patients for Month 6 (5.4±3.57). The available data was incomplete for subsequent months: Month 7 (n=11; 7.8±3.03), Month 8 (n=10; 7.4±3.60), Month 9 (n=6; 8.2±3.25), Month 10 (n=3; 11.7±3.06), Month 11 (n=2; 12.5±4.95). Month 12 had a single patient with a change from baseline of 16.0. Fine motor skills continued to improve in these patients as predicted by natural history studies. Chabanon et al., “Prospective and longitudinal natural history study of patients with Type 2 and 3 spinal muscular atrophy: Baseline data NatHis-SMA study.” (2018) PLoS ONE. 13(7): e0201004.

The change from baseline in the gross motor subtest was available for all 13 patients for Month 6 (3.8±5.01). The available data was incomplete for subsequent months: Month 7 (n=12; 4.7±4.29), Month 8 (n=10; 4.9±6.45), Month 9 (n=6; 3.5±2.07), Month 10 (n=3; 5.7±4.73), Month 11 (n=2; 8.0±4.24), and Month 12 (n=1; 11.0). Patients were continuing to gain gross motor milestones. No patient had lost milestones.

Dose C (2.4×10¹⁴ vg): Limited data for the change from baseline in the fine motor subtest was available: Month 2 (n=3; 0.7±0.58), Month 3 (n=2; 3.5±0.71); Month 4 had a single patient with a change from baseline of 6.0. The change from baseline in the gross motor subtest was available up to 4 months: Month 2 (n=3; 0.3±1.53), Month 3 (n=2; 0.5±3.54), and Month 4 (n=1; 4.0).

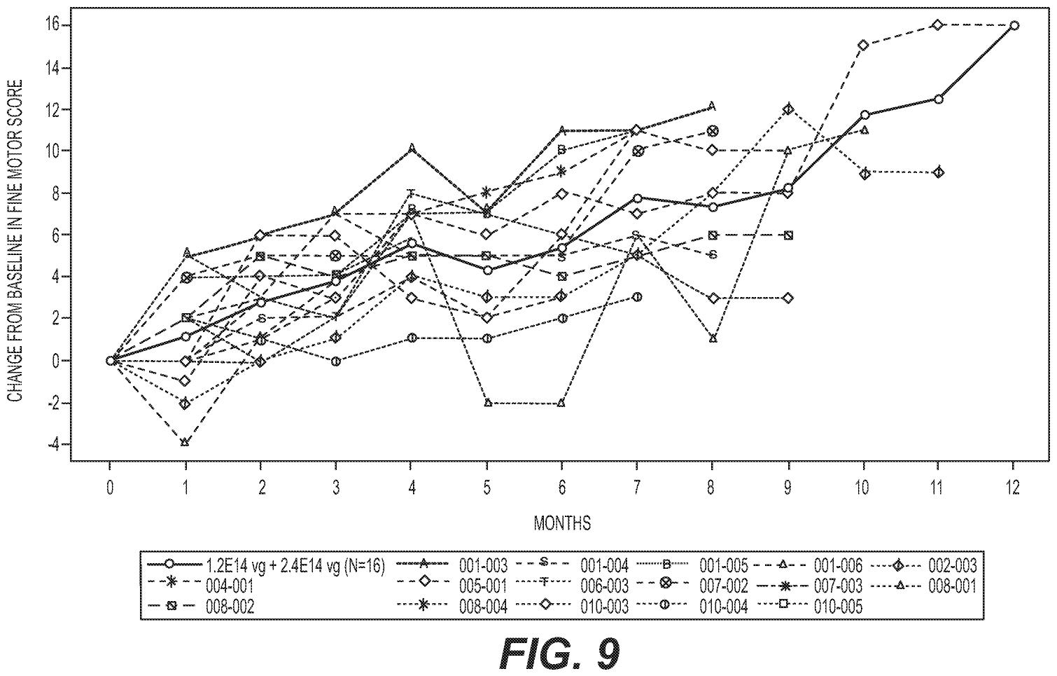

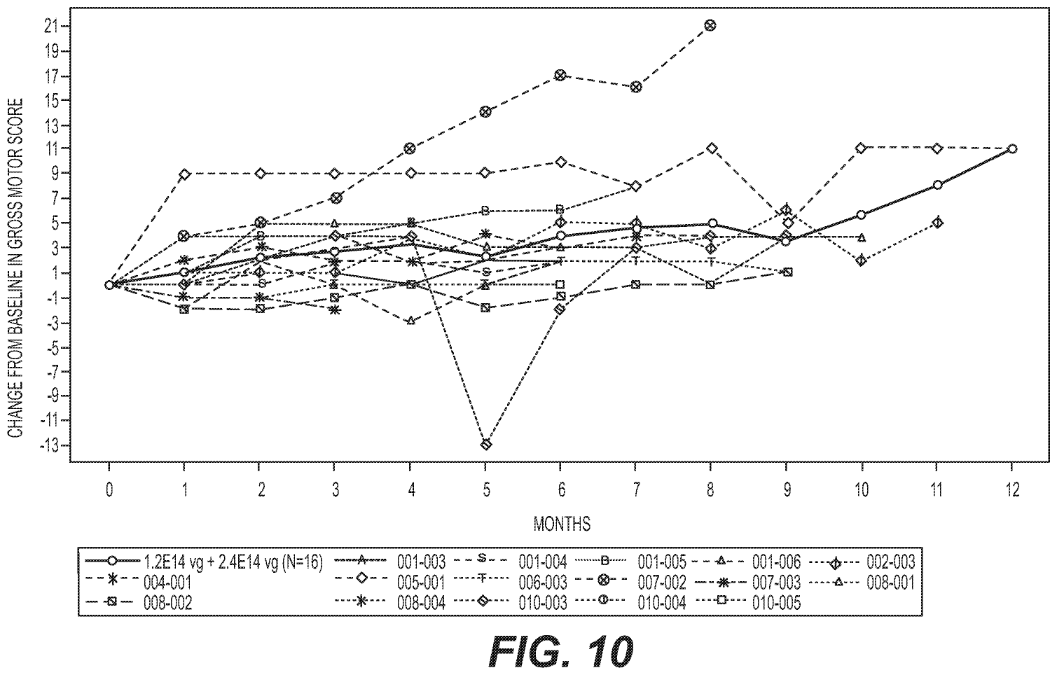

Dose B+Dose C: The spaghetti plot for the change from baseline in Bayley Scales® up to 12 months for Dose B+Dose C is given in FIG. 9 (Fine Motor) and FIG. 10 (Gross Motor). Descriptive statistics for the Bayley Scales® are provided in Table 14.

TABLE 14
Analysis on maximum change from baseline in gross and fine motor scores of
Bayley Scale for Infant and Toddler Development ® at any post-baseline
visit up to 12 months for patients <24 months of age at time of dosing - ITT Set

| | | | | |
|---|---|---|---|---|
| Category Visit | Dose A | Dose B | Dose C | Dose B + C |
| Statistics | (N = 3) | (N = 13) | (N = 3) | (N = 16) |

Gross Motor
Baseline

| n | 3 | | 13 | | 3 | | 16 | |
|---|---|---|---|---|---|---|---|---|
| Mean (SD) | 26.3 | (8.62) | 20.8 | (4.46) | 25.0 | (7.00) | 21.6 | (5.03) |
| Median (Min, Max) | 28.0 | (17, 34) | 20.0 | (14, 3) | 25.0 | (18, 32) | 20.0 | (14, 32) |

Post-baseline value for the visit with maximum CFB observed value

| n | 3 | | 13 | | 3 | | 16 | |
|---|---|---|---|---|---|---|---|---|
| Mean (SD) | 32.0 | (7.55) | 26.3 | (8.48) | 26.0 | (5.29) | 26.3 | (7.83) |
| Median (Min, Max) | 33 | (24, 39) | 24.0 | (18, 51) | 24.0 | (22, 32) | 24.0 | (18, 51) |

Change from baseline

| n | 3 | | 13 | | 3 | | 16 | |
|---|---|---|---|---|---|---|---|---|
| Mean (SD) | 5.7 | (1.15) | 5.5 | (5.43) | 1.0 | (2.65) | 4.7 | (5.28) |
| Median (Min, Max) | 5.0 | (5, 7) | 4.0 | (1, 21) | 0.0 | (−1, 4) | 4.0 | (−1, 21) |

Fine Motor
Baseline

| n | 3 | | 13 | | 3 | | 16 | |
|---|---|---|---|---|---|---|---|---|
| Mean (SD) | 31.3 | (2.89) | 31.2 | (4.64) | 36.0 | (6.08) | 32.1 | (5.08) |
| Median (Min, Max) | 33.0 | (28, 33) | 31.0 | (22, 38) | 33.0 | (32, 43) | 31.5 | (22, 43) |

Post-baseline value for the visit with maximum CFB observed value

| n | 3 | | 13 | | 3 | | 16 | |
|---|---|---|---|---|---|---|---|---|
| Mean (SD) | 46.7 | (5.03) | 40.5 | (5.97) | 39.0 | (3.61) | 40.3 | (5.53) |
| Median (Min, Max) | 46.0 | (42, 52) | 41.0 | (32, 50) | 38.0 | (36, 43) | 40.0 | (32, 50) |

Change from baseline

| n | 3 | | 13 | | 3 | | 16 | |
|---|---|---|---|---|---|---|---|---|
| Mean (SD) | 15.3 | (5.51) | 9.3 | (3.75) | 3.0 | (3.00) | 8.1 | (4.35) |
| Median (Min, Max) | 18.0 | (9, 19) | 11.0 | (3, 16) | 3.0 | (0, 6) | 9.0 | (0, 16) |

b. Patients Aged ≥4 Months and <60 Months at Time of Dosing

The ≥24 and <60 months age group is composed of 12 patients who received Dose B (1.2×10¹⁴ vg). Gains in fine and gross motor subsets were observed. The change from baseline in the fine motor subtest was available for all 12 patients for Month 6 (7.6±5.62). The available data were incomplete for subsequent months: Month 7 (n=11; 6.6±5.33), Month 8 (n=11; 8.0±5.74), Month 9 (n=10; 7.9±5.53), and Month 10 (n=2; 10.5±0.71). Single patients had data at Month 11 (n=1) and Month 12 (n=1) with scores of 9.0, and 10.0, respectively.

For the gross motor subset, the change from baseline was available for all 12 patients for Month 6 (1.8±4.47). The available data were incomplete for subsequent months: Month 7 (n=11; 2.0±4.36), Month 8 (n=11; 2.3±4.47), Month 9 (n=10; 2.4±5.08), Month 10 (n=2; 5.5±6.36). No patient lost Bayley® gross motor milestones.

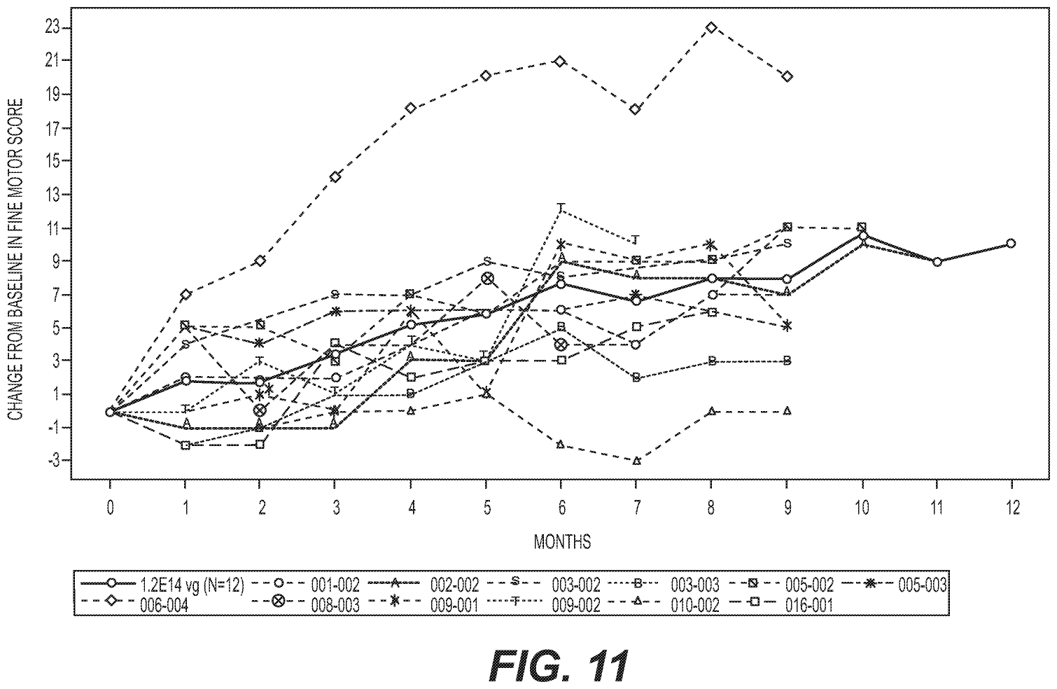

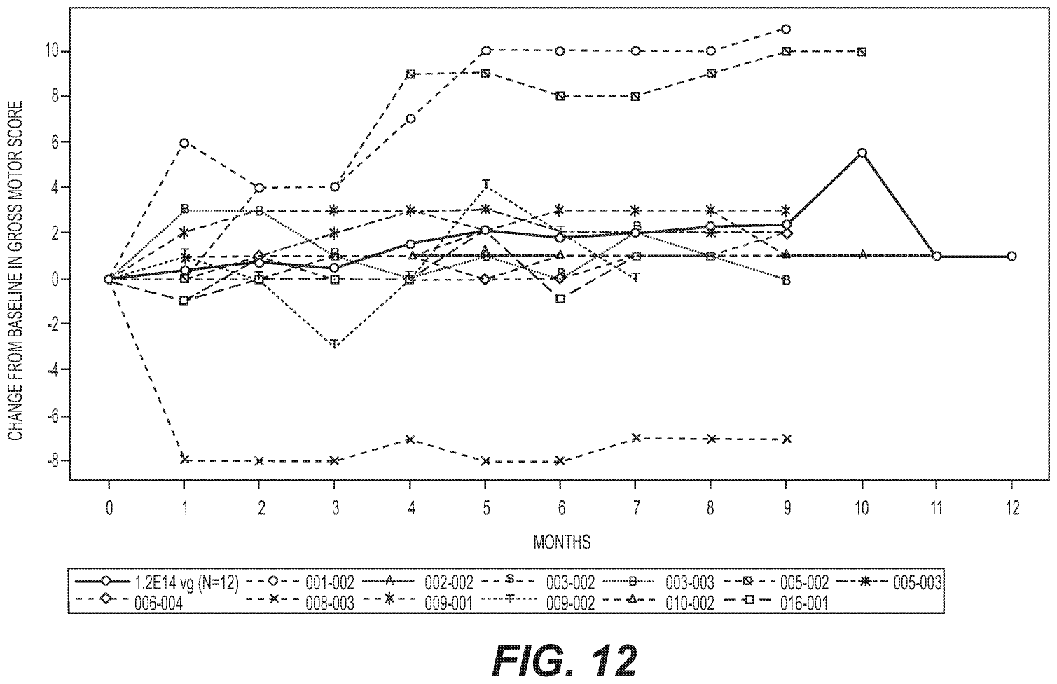

The spaghetti plot for the change from baseline in Bayley Scales® up to 12 months for Dose B is given in FIG. 11 and FIG. 12. The curve for patient 008-003 is incorrect. The baseline score for patient 008-003 was 20, not 28 (as initially reported). Therefore, the change in Gross Motor Score between the baseline measurement and Month 1 was “0”, not “−8”. In addition, patient 008-003's change in Gross Motor Score from the baseline measurement was “0” for Months 2 and 3, “+1” for Month 4, “0” for Months 5 and 6, “+1” for Months 7-11, and “+2” for Month 12.

These interim data summarize the efficacy results from the clinical trial described in Example 1 as of 12 months post-treatment. Descriptive statistics for the Bayley Scales® are provided in Table 15.

TABLE 15
Analysis of maximum change from baseline in gross and fine
motor scores of Bayley Scales for Infant and Toddler Development ® at
any post-baseline visit up to 12 months for patients ≥24
and <60 months of age at time of dosing - ITT Set

| | |
|---|---|
| | Dose B |
| Category Visit Statistics | (N = 12) |

Gross Motor
Baseline

| n | 12 | |
|---|---|---|
| Mean (SD) | 23.2 | (6.15) |
| Median (Min, Max) | 20.5 | (16, 35) |

Post-baseline value for the visit with maximum CFB observed value

| n | 12 | |
|---|---|---|
| Mean (SD) | 26.2 | (6.83) |
| Median (Min, Max) | 24.5 | (18, 38) |

Change from baseline

| n | 12 | |
|---|---|---|
| Mean (SD) | 3.0 | (4.51) |
| Median (Min, Max) | 3.0 | (−7, 11) |

Fine Motor
Baseline

| n | 12 | |
|---|---|---|
| Mean (SD) | 46.2 | (8.77) |
| Median (Min, Max) | 47 | (32, 60) |

Post-baseline value for the visit with maximum CFB observed value

| n | 12 | |
|---|---|---|
| Mean (SD) | 55.6 | (5.66) |
| Median (Min, Max) | 55.0 | (46, 65) |

Change from baseline

| n | 12 | |
|---|---|---|
| Mean (SD) | 9.4 | (5.32) |
| Median (Min, Max) | 10.0 | (1, 23) |

### Interim Results: Change in HFMSE Scores Among Patients ≥6 Months and <24 Months of Age Who Continue in the Study Past 24 Months of Age

HFMSE scoring was recorded for those patients in the patients ≥6 and <24 months age group who reached 24 months of age. Since a pre-treatment baseline was not available for any patient, the first record of HFMSE is defined as the baseline. The month designations below are relative to the first record of HFMSE at 24 months of age, not the study month.

Dose A (6.0×10¹³ vg): Two patients reached 24 months of age. The change from the first record of HFMSE is provided: Month 1 (n=2; −0.5±4.95), Month 2 (n=2; 4.0±0.00), Month 3 (n=2; 3.5±0.71), Month 4 (n=2; 3.0±2.83), Month 5 (n=1; 5.0), and Month 6 (n=2; 2.0±5.66).

Dose B (1.2×10¹⁴ vg): Eight patients reached 24 months of age. The change from the first record of HFMSE is provided: Month 1 (n=7; 2.0±2.83), Month 2 (n=7; 2.7±2.69), Month 3 (n=6; 1.3±4.97), Month 4 (n=3; 4.7±4.51), and Month 5 (n=2; 7.5±0.71).

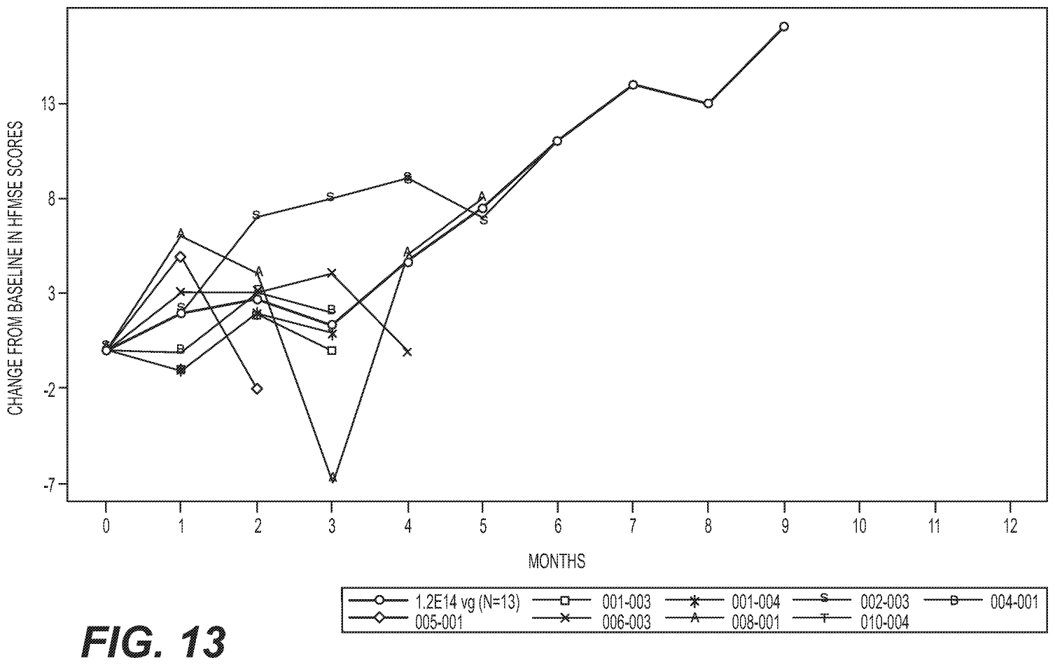

The spaghetti plot for the change from baseline in HFMSE scores up to 12 months for Dose B is given in FIG. 13. The maximum change (mean±SD) from baseline in HFMSE values at any post-baseline visit up to 12 months for Dose B was 17.7±5.28 (n=7) as shown in Table 16.

Dose C (2.4×10¹⁴ vg): A single patient reached the first record of HFMSE at ≥24 months of age. Only this single “baseline” data point was available.

TABLE 16
Maximum change from baseline in HFMSE at any post-baseline visit
up to 12 months for patients <24 months at time of dosing
who continue in the study past 24 months of age - ITT set

| | | |
|---|---|---|
| Category Visit | Dose B | Dose C |
| Statistics | (N = 13) | (N = 3) |

Baseline defined as first HFMSE assessment during the study
when patients reach 24 months of age

| n | 8 | | 1 |
|---|---|---|---|
| Mean (SD) | 13.0 | (5.61) | 33.0 |
| Median (Min, Max) | 13.0 | (6, 21) | 33.0 (33, 33) |

Post-baseline value for the visit with maximum CFB observed value

| n | 7 | | 0 |
|---|---|---|---|
| Mean (SD) | 17.7 | (5.28) | — |
| Median (Min, Max) | 17 | (11, 25) | — |

Change from baseline

| n | 7 | | 0 |
|---|---|---|---|
| Mean (SD) | 5.9 | (5.34) | — |
| Median (Min, Max) | 4.0 | (2, 17) | — |

### Interim Conclusions

The clinical trial described herein is an ongoing Phase 1, open-label, single-dose intrathecal (IT) administration study of infants and children ≥6 months and <60 months of age who are diagnosed with spinal muscular atrophy (SMA). The data obtained so far in the treated patients show clinically meaningful changes in motor function that include advancing skills, advancing milestones, and disease stabilization which is described in the summaries of each age group below.

### ≥6 Months and <24 Months Age Group

Nineteen patients ≥6 months and <24 months of age were enrolled to the clinical trial. Three patients received a single dose of 6.0×10¹³ vg of AVXS-101 (Dose A), 13 patients received a single dose of 1.2×10¹⁴ vg of AVXS-101 (Dose B), and 3 patients received a single dose of 2.4×10¹⁴ vg of AVXS-101 (Dose C). Four patients completed the 12-month post-dose assessments: 3 patients in the Dose A group and 1 patient in the Dose B group.

The primary efficacy endpoint for this age group was attainment of Bayley Scales of Infant and Toddler Development®—Gross Motor Subset #40, “stand without support for at least 3 seconds”. Two patients achieved primary efficacy endpoints. Patient 007-001 who received Dose A achieved standing without support for at least 3 seconds at 11 months post-treatment. Patient 007-002, who received Dose B, achieved standing without support by 3 months post-treatment.

The secondary efficacy endpoint was the Bayley Scales of Infant and Toddler Development®—Gross Motor Subset #43 (“walks independently steps”). One patient (007-002) who received Dose B walked without assistance for at least 5 steps at 4 months post-treatment.

The exploratory endpoint was the change from baseline in fine and gross motor components of the Bayley Scales of Infant and Toddler Development®, Third Edition (Bayley®-III). Since the Bayley Scales® were not assessed in the PNCR dataset, only descriptive statistics are provided for patients <24 months of age. However, patients are continuing to gain gross motor milestones. No patient has lost milestones.

### ≥24 Months and <60 Months Age Group

Twelve patients ≥24 months and <60 months of age were enrolled to the clinical trial and received Dose B. No patients in this age group received Dose C. A single patient completed the 12-month post-treatment assessments.

The primary efficacy endpoint for this age group was the change from baseline in HFMSE. To place the changes observed in the Dose B group into context, a 3-point improvement in HFMSE score is considered meaningful and important to stakeholders such as caregivers and clinicians and is used as the threshold for detecting meaningful change in clinical trials. Mercuri et al., “Nusinersen versus sham control in later-onset spinal muscular atrophy.” N Engl J Med. 378(7): 625-635. The Dose B treatment group showed a robust increase in HFMSE scores over the PNCR N=15 natural history control group. For the PNCR N=15 natural history control group, maximum change in HFMSE score was observed at Month 9 (n=7) of 1.0±2.16. Similar results were observed when performing the Sensitivity Analysis using the PNCR N=17 natural history control group with a maximum change in HFMSE score at Month 9 (n=8) of 1.1±2.03.

The Dose B treatment group showed a clinically meaningful increase of 5.7±6.72 for the change in HFMSE score at Month 9 (n=10).

The exploratory endpoint was the change from baseline in fine and gross motor components of the Bayley®-III. Similar to the younger age group, patients are continuing to gain gross motor milestones. No patient has lost milestones.

1-118. (canceled)

119. A method of treating spinal muscular atrophy (SMA) in a patient in need thereof, comprising administering intrathecally an AAV9 viral vector comprising a polynucleotide encoding a survival motor neuron (SMN) protein, wherein the AAV9 viral vector is administered at a dose of about 1×10¹³ vg-5×10¹⁴ vg.

120. The method of claim 119, wherein the AAV9 viral vector further comprises a modified AAV2 ITR, a chicken beta-actin (CB) promoter, a cytomegalovirus (CMV) immediate/early enhancer, a modified SV40 late 16S intron, a bovine growth hormone (BGH) polyadenylation signal, and an unmodified AAV2 ITR.

121. The method of claim 119, wherein the SMN protein comprises an amino acid sequence of SEQ ID NO: 2 and/or the AAV9 viral vector comprises the nucleic acid sequence of SEQ ID NO: 1.

122. The method of claim 119, wherein the AAV9 vector comprises a capsid comprising the amino acid sequence of SEQ ID NO: 3.

123. The method of claim 119, further comprising a pharmaceutically acceptable carrier suitable for intrathecal administration.

124. The method of claim 119, wherein the AAV9 viral vector is administered together with a contrast medium.

125. The method of claim 124, wherein the contrast medium comprises iohexol.

126. The method of claim 119, wherein the AAV9 viral vector is administered at a dose of about 1.2×10¹⁴ vg.

127. The method of claim 119, wherein the AAV9 viral vector is administered at a dose of about 2.4×10¹⁴ vg.

128. The method of claim 119, wherein the SMA is Type II SMA or Type III SMA.

129. The method of claim 119, wherein the patient at the time of administration of the AAV9 viral vector is:
a) six months of age or older;
b) between six months and twenty-four months of age;
c) between twenty-four months and sixty months of age;
d) twenty-four months of age or younger; or
e) sixty months of age or younger.

130. The method of claim 119, wherein the patient prior to or at the time of administration of the AAV9 viral vector:
a) has bi-allelic SMN1 null mutations or inactivating deletions;
b) has a deletion of exon seven of SMN1;
c) has three or more copies of SMN2;
d) does not have a c.859G>C substitution in exon seven on at least one copy of the SMN2 gene;
e) shows onset of disease before about 12 months of age;
f) has the ability to sit unassisted at the time of the AAV9 viral vector administration, as defined by the World Health Organization Multicentre Growth Reference Study (WHO-MGRS) criteria, for about 10 or more seconds, but cannot stand or walk;
g) has one or more of gamma-glutamyl transferase levels less than about 3 times upper limit of normal, bilirubin levels less than about 3.0 mg/dL, creatinine levels less than about 1.0 mg/dL, Hgb levels between about 8-18 g/dL, and/or white blood cell counts of less than about 20000 per mm³;
h) has platelet counts above about 67,000 cells/ml, or above about 100,000 cells/ml, or above about 150,000 cells/ml;
i) has normal hepatic function;
j) has hepatic transaminase levels less than about 8-40 U/L; and/or
k) has anti-AAV9 antibody titers at or below 1:25, 1:50, 1:75, or 1:100, as determined by an Enzyme-linked Immunosorbent Assay (ELISA).
l) does not have severe scoliosis (defined as 50° curvature of spine) evident on X-ray examination;
m) is not contraindicated for spinal tap procedure or administration of intrathecal therapy;
n) has not previously had a scoliosis repair surgery or procedure;
o) does not need the use of invasive ventilatory support;
p) does not have a history of standing or walking independently;
q) does not use a gastric feeding tube;
r) does not have an active viral infection;
s) has not had a severe non-pulmonary and/or respiratory tract infection within four weeks;
t) does not have concomitant illness, major renal or hepatic impairment, known seizure disorder, diabetes mellitus, idiopathic hypocalciuria or symptomatic cardiomyopathy;
u) does not have a history of bacterial meningitis or brain or spinal cord disease;
v) does not have a known allergy or hypersensitivity to prednisolone or other glucocorticosteroids or excipients;
w) does not have a known allergy or hypersensitivity to iodine or iodine-containing products;
x) is not taking drugs to treat myopathy or neuropathy;
y) is not receiving immunosuppressive therapy, plasmapheresis, immunomodulators or adalimumab, within three months; and/or
z) has not received an investigational or approved compound product or therapy to treat SMA.

131. The method of claim 119, wherein the patient is placed in the Trendelenburg position during and/or after administration of the AAV9 viral vector.

132. The method of claim 119, wherein the patient is administered an oral steroid at least about 1-48 hours prior to being administered the AAV9 viral vector and the patient:
a) is administered the oral steroid at a dose of about 1 mg/kg;
b) is administered the oral steroid once or twice daily;
c) is administered the oral steroid at a dose of about 1 mg/kg and then tapered down to 0.5 mg/kg/day for 2 weeks after administration of the AAV9 viral vector, followed by 0.25 mg/kg/day for 2 more weeks;
d) is administered the oral steroid for at least about 10-60 days after being administered the AAV9 viral vector;
e) continues to receive the oral steroid more than 30 days after AAV9 viral vector administration and until aspartate transaminase (AST) and/or alanine aminotransferase (ALT) levels are below twice the upper limit of normal or below about 120 IU/L;
f) continues to receive the oral steroid more than 30 days after AAV9 viral vector administration and until a T cell response in a blood sample from the patient falls below 100 spot forming cells (SFC) per 10⁶ peripheral blood mononuclear cells (PBMCs); and/or
g) is administered the oral steroid until the patient's anti-AAV9 antibody titers decrease to below 1:25, 1:50, 1:75, or 1:100, as determined by an ELISA.

133. The method of claim 132, wherein the oral steroid is prednisolone or an equivalent.

134. The method of claim 119, further comprising administering a second therapeutic agent to the patient concomitantly or consecutively with the administration of the AAV9 viral vector.

135. The method of claim 134, wherein the second therapeutic agent comprises an antisense oligonucleotide targeting SMN1 and/or SMN2, a muscle enhancer, and/or a neuroprotector.

136. The method of claim 119, wherein, after administration of the AAV9 viral vector, the patient:
a) does not have severe scoliosis (defined as 50° curvature of spine) evident on X-ray examination;
b) does not have a scoliosis repair surgery or procedure within 6 months to 3 years;
c) does not need the use of invasive ventilatory support;
d) does not use a gastric feeding tube;
e) has anti-AAV9 antibody titers at or above 1:25, 1:50, 1:75, or 1:100, as determined by an ELISA, and is monitored for about 1-8 weeks or until titers decrease to below 1:25, 1:50, 1:75, or 1:100;
f) has platelet counts below about 67,000 cells/ml, or below about 100,000 cells/ml, or below about 150,000 cells/ml, and is monitored for about 1-8 weeks or until platelet counts increase to about 67,000 cells/ml, or above about 100,000 cells/ml, or above about 150,000 cells/ml; and/or
g) has platelet counts below about 67,000 cells/ml and is treated with a platelet transfusion.

137. The method of claim 119, wherein, 1-24 months after administration of the AAV9 viral vector, the patient achieves:
a) the ability to stand without support for at least about three seconds, as defined by the Bayley Scales of Infant and Toddler Development®;
b) the ability to walk without assistance, as defined by the Bayley Scales of Infant and Toddler Development®;
c) the ability to take at least five steps independently, as defined by the Bayley Scales of Infant and Toddler Development®;
d) a change after treatment from a baseline measurement at time of treatment, as defined by the Bayley Scales of Infant and Toddler Development®; and/or
e) an improvement of at least three points in the Gross Motor component of the Bayley Scales of Infant and Toddler Development® relative to a pre-administration score.

138. The method of claim 119, wherein the AAV9 viral vector is administered at a dose of about 6×10¹³ vg-2.4×10¹⁴ vg, and wherein the patient achieves an improved score on the Hammersmith Functional Motor Scale-Expanded relative to a pre-administration score; and/or the patient achieves an improved score on the Bayley Scales of Infant and Toddler Development®, relative to a pre-administration score.

139. The method of claim 138, wherein the administration results in:
a) an improvement of at least three points on the Hammersmith Functional Motor Scale-Expanded by 9 months after administration relative to a pre-administration score;
b) an improvement of at least four points on the Hammersmith Functional Motor Scale-Expanded by 9 months after administration relative to a pre-administration score;
c) an improvement of at least five points on the Hammersmith Functional Motor Scale-Expanded by 9 months after administration relative to a pre-administration score;
d) an improvement of at least three points in the Gross Motor component of the Bayley Scales of Infant and Toddler Development® after administration relative to a pre-administration score;
e) the ability to stand without support for at least three seconds by 12 months after administration; and/or
f) the ability to walk independently for at least five steps by 12 months after administration.

140. A pharmaceutical composition comprising an AAV9 viral vector comprising a modified AAV2 ITR, a chicken beta-actin (CB) promoter, a cytomegalovirus (CMV) immediate/early enhancer, a modified SV40 late 16S intron, a polynucleotide encoding a survival motor neuron (SMN) protein, a bovine growth hormone (BGH) polyadenylation signal, and an unmodified AAV2 ITR, wherein the AAV9 viral vector is present in the pharmaceutical composition at a dose of about 1.2×10¹⁴ vg or about 2.4×10¹⁴ vg.

141. The pharmaceutical composition of claim 140, wherein the SMN protein comprises an amino acid sequence of SEQ ID NO: 2 and/or the AAV9 viral vector comprises the nucleic acid sequence of SEQ ID NO: 1.

142. The pharmaceutical composition of claim 140, wherein the AAV9 viral vector is encapsidated in an AAV9 virion.

143. The pharmaceutical composition of claim 142, wherein the AAV9 virion comprises a capsid comprising the amino acid sequence of SEQ ID NO: 3.

144. The pharmaceutical composition of claim 140, further comprising a contrast medium.

145. The pharmaceutical composition of claim 144, wherein the contrast medium comprises iohexol.

146. The pharmaceutical composition of claim 140, wherein the composition is in a container and comprises at least one of the following:
a) a pH of about 7.7-8.3,
b) an osmolarity of about 390-430 mOsm/kg,
c) less than about 600 particles that are 25 μm in size per container,
d) less than about 6000 particles that are 10 μm in size per container,
e) a genomic titer of about 1.7×10¹³-5.3×10¹³ vg/mL,
f) an infectious titer of about 3.9×10⁸-8.4×10¹⁰ IU per 1.0×10¹³ vg,
g) a total protein of about 100-300 μg per 1.0×10¹³ vg,
h) a Poloxamer 188 content of about 20-80 ppm,
i) a relative potency of about 70-130% based on an in vitro cell-based assay, wherein the potency is relative to a reference standard and/or suitable control,
j) a potency characterized by median survival in a SMNΔ7 mouse model of greater than or equal to 24 days at a dose of 7.5×10¹³ vg/kg,
k) less than about 5% empty capsid,
l) a total purity of greater than or equal to about 95%,
m) less than or equal to about 0.13 EU/mL endotoxin,
n) less than about 0.09 ng of benzonase per 1.0×10¹³ vg,
o) less than about 30 μg/g (ppm) of cesium,
p) less than about 0.22 ng of bovine serum albumin (BSA) per 1.0×10¹³ vg,
q) less than about 6.8×10⁵ μg of residual plasmid DNA per 1.0×10¹³ vg,
r) less than about 1.1×10⁵ μg of residual hcDNA per 1.0×10¹³ vg, and
s) less than about 4 ng of rHCP per 1.0×10¹³ vg.
